(19) United States
(12) Patent Application Publication
(10) Pub. No.: US 2005/0260601 A1
(43) Pub. Date: Nov. 24, 2005

(54) Recombinant mutants of rhabdovirus and methods of use thereof

(76) Inventors: Michael A. Whitt, Cordova, TN (US); Clinton S. Robison, Pittsburgh, PA (US); Himangi R. Jayakar, Cordova, TN (US); Mark A. Miller, Germantown, TN (US)

Correspondence Address: EITAN, PEARL, LATZER & COHEN ZEDEK LLP, 10 ROCKEFELLER PLAZA, SUITE 1001, NEW YORK, NY, 10020, US

(21) Appl. No.: 10/656,894

(22) Filed: Sep. 8, 2003

Related U.S. Application Data
(60) Provisional application No. 60/408,908, filed on Sep. 9, 2002.

(51) Int. Cl. C12Q 1/68; G01N 33/574

(52) U.S. Cl. 435/6; 435/7.23

(57) ABSTRACT

The present invention relates to recombinant Rhabdoviridae, isolated nucleic acids, vectors, cells and compositions comprising same. The recombinant Rhabdoviridae, isolated nucleic acids, vectors, cells and compositions express Rhabdoviral proteins including a mutated matrix protein (M) and/or a mutated glycoprotein (G), in addition to expression of at least one foreign nucleic acid. The present invention also relates to methods of use thereof, including their use in vivo, in anti-cancer applications, such as in the treatment of gliomas. The recombinant Rhabdoviridae of the present invention are also useful in gene therapy and vaccine applications.

### CROSS REFERENCE TO RELATED APPLICATION

This Application claims priority from U.S. Provisional Application Ser. No. 60/408,908, which was filed on Sep. 9, 2002, which is hereby incorporated by reference.

### FIELD OF THE INVENTION

The present invention relates to recombinant Rhabdoviridae, expressing Rhabdoviral proteins including a mutated matrix protein (M) and/or a mutated glycoprotein (G), in addition to expression of at least one foreign nucleic acid, contained in their genome. The present invention also relates to methods of use thereof, including their use in vivo, in anti-cancer applications, such as in the treatment of gliomas. The recombinant Rhabdoviridae of the present invention are also useful in gene therapy and vaccine applications.

### BACKGROUND OF THE INVENTION

Despite enormous breakthroughs in the development of appropriate vectors for gene delivery in therapeutic applications, numerous obstacles remain, in particular in the development of effective delivery systems for gene therapy and vaccine development, and their impact on anti-cancer therapies.

Gene therapy viral vectors typically do not lyse the cells they target. Viral vectors used for gene therapy are engineered to deliver therapeutically effective DNAs with relative safety, like a drug (see for example, D. T. Curiel et al., U.S. Pat. No. 5,547,932). Some of these vectors are capable of replicating upon infection, but only within targeted cells (F. McConnick, U.S. Pat. No. 5,677,178). Other gene therapy vectors are engineered such that they are unable to replicate. Non-replicating gene therapy vectors are usually produced using helper plasmids (see for example, G. Natsoulis, U.S. Pat. No. 5,622,856; M. Mamounas, U.S. Pat. No. 5,646,034) or packaging cells that confer genetic elements missing in the virus genome.

Wide application of viral gene therapy vectors has been hampered by the fact that wild-type tropisms natural to the viral vector being utilized cannot often be easily overcome. In recent years, many gene therapy patents have been issued describing adenoviral vectors (M. Cotten et al., U.S. Pat. No. 5,693,509); adeno-associated viral vectors (J. S. Lebkowski et al., U.S. Pat. No. 5,589,377); retroviral vectors (B. O. Palsson et al., U.S. Pat. No. 5,616,487); vectors containing chimeric fusion glycoproteins (S. Kayman et al., U.S. Pat. No. 5,643,756); vectors that contain an antibody to a viral coat protein (Cotten et al.); hybrid viruses engineered to allow infection with human immunodeficiency type 1 (HIV-1) in monkeys, a species that normally cannot be infected by HIV-1 (J. Sodroski et al., U.S. Pat. No. 5,654,195); and pseudotype retroviral vectors which contain the G protein of Vesicular Stomatitis Virus (VSV) (J. C. Bums et al., U.S. Pat. Nos. 5,512,421 and 5,670,354). Some modifications of the gene therapy vectors attempt to overcome tropism-limiting aspects inherent to the individual vectors, while maintaining the efficacy of the vector for use in gene therapy. Virus delivery vehicles have also been created for transient gene therapy; wherein expression of the gene delivered to the cell is not permanent (I. H. Maxwell et al., U.S. Pat. No. 5,585,254).

Vaccine development and the promotion of effective immune responses is another field in biomedical research that would benefit from better design of appropriate gene delivery systems, in particular in terms of viral delivery vehicles. It has been well documented that the cytokines produced during the initial stages of the immune response to an invading pathogen or vaccine formulation play a critical role in the development of antigen-specific Th cells. Several lines of evidence demonstrated that the “decision” of T helper cell differentiation to a phenotype associated with protection is strongly influenced by the cytokine milieu in which the T helper cells are found (1). Moreover, many different cytokines have been shown to have immunomodulatory effects that can promote the development of cell-mediated, antigen-specific immune responses when administered as a therapeutic or as an adjuvant component. In order to take full advantage of the immunomodulatory and adjuvant properties of such cytokines, many researchers have begun to evaluate the use of vectors such as plasmid DNA (2), engineered cells (3,4), or recombinant viruses (5) to deliver quantities of these cytokines in vivo.

Other fields in which viral vectors, in particular, have shown promise is in applications in the treatment of tumors, in particular in the treatment of brain tumors. The rapid advances made in cancer gene therapy have renewed the hope that such technologies can provide a successful adjuvant to surgery. Two therapeutic strategies for treating cancers with these tools have been developed. One approach, termed virotherapy or oncolytic therapy, utilizes the inherent destructive capacity of cytolytic viruses to kill tumor cells. These so-called oncolytic viruses are genetically modified so that they specifically target tumor cells or are replication-restricted in normal tissues and thereby preferentially destroy tumor cells. One example of a replication-restricted oncolytic virus is ONYX-015. ONYX-015 is an adenovirus that has the E1B gene deleted and that is replication-restricted in normal cells with a wild-type p53 gene, but that replicates and kills tumor cells lacking a functional p53 (6). Another approach involves the delivery of therapeutic or cytotoxic genes to tumor cells. The products of these genes either directly or indirectly inhibit tumor growth. A number of different genes have been tested in preclinical and clinical studies, including human cytokine genes, tumor suppressor genes, bacterial or viral prodrug-activating enzyme encoding genes (suicide genes) and genes which make the tumor mass more susceptible to radiation and chemotherapy.

Rhabdoviridae are membrane-enveloped viruses that are widely distributed in nature where they infect vertebrates, invertebrates, and plants. Vesicular stomatitis virus (VSV) is part of the Rhabdoviridae viral family, which is divided into 6 genera in which the VSV is one of them. Rhabdoviridae have single, negative-strand RNA genomes of 11-12,000 nucleotides (Rose and Whitt, 2001, Chapter 38, Rhabdoviridae: The viruses and their replication, in Fields Virology, 4^{th} edition, pp. 1221-1244.). Viral particles contain a helical, nucleocapsid core composed of genomic RNA and protein. Generally, three proteins, termed N (nucleocapsid, which encases the genome tightly), P (formerly termed NS, originally indicating nonstructural), and L (large) are found to be associated with the nucleocapsid. An additional matrix (M) protein lies within the membrane envelope, perhaps interacting both with the membrane and the nucleocapsid core. A single glycoprotein (G) species spans the membrane and forms the spikes on the surface of the virus particle. G is responsible for binding to cells and membrane fusion. Because the genome is the negative sense [i.e., complementary to the RNA sequence (positive sense) that functions as mRNA to directly produce encoded protein], Rhabdoviridae must encode and package an RNA-dependent RNA polymerase in the virion (Baltimore et al., 1970, Proc. Natl. Acad. Sci. USA 66: 572-576), composed of the P and L proteins. This enzyme transcribes genomic RNA to make subgenomic mRNA's encoding the 5-6 viral proteins and also replicates full-length positive and negative sense RNAs. The genes are transcribed sequentially, starting at the 3′ end of the genomes.

The matrix protein of VSV serves two critical functions in the life cycle of the virus. First, it is essential for virus assembly and the release of virus particles from infected cells. Second, it is responsible for the inhibition of host cell gene expression, which allows the virus to utilize all of the host cell translation machinery for synthesis of viral proteins. The inhibition of host gene expression by M protein is thought to be responsible for the severe and rapid cytopathic effects associated with VSV infections. The M protein-induced cytopathic effect causes the induction of apoptosis and typically results in cell death within 12 to 16 hours post-infection. Transient expression of M protein alone from a eukaryotic expression vector is sufficient to induce the typical VSV cytopathic effects, which includes disassembly of the host cell cytoskeleton and cell rounding, demonstrating that no other VSV proteins are required for VSV-induced cytopathic effects.

The VSV G protein mediates both virus attachment to the host cell as well as fusion of the viral envelope with the endosomal membrane following endocytosis. Results of mutational analyses of residues 118-136 of the G protein ectodomain as well as results from hydrophobic photolabeling experiments with VSV provided evidence that this region is the internal fusion peptide and that it inserts into target membranes at acidic pH (9-14). It has also been shown that insertions or substitutions in the region between residues 395-418 affect membrane fusion activity of G protein (15,16). Double mutants with substitutions in both the fusion peptide and residues 395-418 had an additive effect upon fusion inhibition (17) indicating that the C-terminal region of the ectodomain specfically plays an important role in the fusion activity of G.

There is thus a recognized need for developing viral vectors, for applications in gene delivery, vaccine development and anti-cancer therapy, as described. The development of VSV based vectors, in particular those which do not have a cytopathic effect, do not undergo extensive cell-to-cell spread, and those that replicate exclusively in the cytoplasm, eliminates many of the concerns associated with viral vector therapy, including the concern over insertional mutagenesis in target cell chromosomes.

### SUMMARY OF INVENTION

The present invention discloses, in one embodiment, recombinant Rhabdoviridae in which the matrix protein M and/or the membrane-proximal ectodomain of the Rhabdoviral glycoprotein (G) is mutated or partially deleted. The invention further provides, in other embodiments, for the use of such recombinant Rhabdoviridae for gene transfer protocols, as vaccines and as anti-cancer therapies.

In one embodiment, the recombinant Rhabdovirus is non-cytopathic and further comprises an insertion of a heterologous nucleic acid sequence encoding a second polypeptide. The second polypeptide may, in one embodiment, be a therapeutic polypeptide, or in another embodiment, be immunogenic.

In another embodiment, this invention provides a method of producing a non-cytopathic recombinant Rhabdovirus comprising a genetically modified nucleic acid encoding Rhabdovirus proteins including a mutation or a deletion within a matrix protein (M) comprising the steps of: (A) inserting into a suitable cell a polynucleotide sequence encoding Rhabdovirus proteins including a mutation or a deletion within the matrix protein (M), a polynucleotide sequence encoding a marker polypeptide and a polycistronic cDNA comprising at least the 3′ and 5′ Rhabdovirus leader and trailer regions containing the cis acting signals for Rhabdovirus replication; (B) culturing the cell under conditions that select for a noncytopathic phenotype of said cell; (C) culturing said cell under conditions that permit production of the recombinant Rhabdovirus, and (D) isolating said non cytopathic recombinant Rhabdovirus.

In another embodiment, this invention provides an isolated nucleic acid molecule comprising a polynucleotide sequence encoding a genome of a non-cytopathic Rhabdovirus, the polynucleotide sequence having a mutation or a deletion in the gene encoding a matrix protein (M).

In another embodiment, this invention provides a recombinant Rhabdovirus comprising a nucleic acid of a Rhabdoviral genome wherein the Rhabdoviral genome comprises a deletion or a mutation within a region encoding a membrane-proximal ectodomain of a Rhabdoviral glycoprotein (G). In another embodiment, the Rhabdoviral genome further comprises a mutation or deletion in a matrix protein M). In one embodiment, accoridng to this aspect of the invention, the Rhabdoviral genome further comprises an insertion of a heterologous nucleic acid sequence encoding a second polypeptide. In one embodiment, the second polypeptide is a therapeutic polypeptide. In other embodiments, the second polypeptide is immunogenic, is a suicide gene or is a marker polypeptide.

In another embodiment, this invention provides a method of producing a recombinant Rhabdovirus comprising a genetically modified nucleic acid encoding Rhabdoviral proteins comprising a deletion or a mutation within a membrane-proximal ectodomain of a glycoprotein (G) comprising the steps of: (A) inserting into a suitable cell a polynucleotide sequence encoding Rhabdovirus proteins including a deletion or a mutation within the membrane-proximal ectodomain of the glycoprotein (G), a polynucleotide sequence encoding a marker polypeptide and a polycistronic cDNA comprising at least the 3′ and 5′ Rhabdovirus leader and trailer regions containing the cis acting signals for Rhabdovirus replication; (B) culturing the cell under conditions that permit production of the recombinant Rhabdovirus, and (C) isolating the recombinant Rhabdovirus.

According to this aspect of the invention, in one embodiment, the method further comprises the step of inserting a heterologous nucleic acid encoding a second polypeptide into said cell. In other embodiments, the second polypeptide is a therapeutic polypeptide, or is immunogenic.

In another embodiment, this invention provides an isolated nucleic acid molecule comprising a polynucleotide sequence encoding a genome of a Rhabdovirus, wherein the polynucleotide sequence has a deletion or a mutation in a gene encoding a membrane-proximal ectodomain of the glycoprotein (G).

In another embodiment, this invention provides a method for treating a subject suffering from a disease associated with a defective gene comprising the step of contacting a target cell of said subject with a therapeutically effective amount of a recombinant non-cytopathic Rhabdovirus, wherein the genome of said Rhabdovirus includes a mutation or a deletion within a region encoding a matrix protein M) and/or a mutation or a deletion in a membrane-proximal ectodomain region of a glycoprotein (G) and a heterologous gene capable of being expressed inside the target cell, thereby treating the disease.

In another embodiment, this invention provides a method for immunizing a subject against a disease comprising the step of contacting a target cell of the subject with a therapeutically effective amount of a recombinant virus, wherein the virus comprises a Rhabdoviral genome, or fragment thereof, said Rhabdoviral genome or fragment thereof including a deletion or a mutation within a region encoding a matrix protein (M) and/or a mutation or a deletion in a membrane-proximal ectodomain region of a glycoprotein (G) and a heterologous gene encoding an immunogenic protein, or peptide fragment, capable of being expressed inside the target cell, thereby immunizing against a disease.

In another embodiment, this invention provides a method for cancer cell lysis, comprising the steps of contacting a cancerous cell with a recombinant Rhabdovirus, wherein said Rhabdovirus comprises (a) a nucleic acid comprising a Rhabdoviral genome, or fragment thereof, wherein said Rhabdoviral genome or fragment thereof comprises a deletion or a mutation within a region encoding a matrix protein (M) and/or a deletion or a mutation within a region encoding the membrane-proximal ectodomain of a Rhabdoviral glycoprotein (G); and (b) a non-Rhabdoviral nucleic acid. In other embodiments, the non-Rhabdoviral nucleic acid encodes for a cytokine or suicide gene.

In another embodiment, this invention provides a method for treating cancer, comprising the steps of contacting a cancerous cell with a recombinant virus, wherein said virus comprises (a) a nucleic acid comprising a Rhabdoviral genome, or fragment thereof, said Rhabdoviral genome or fragment thereof comprises a deletion or a mutation within a region encoding a matrix protein (M) and/or a deletion or a mutation within a region encoding the membrane-proximal ectodomain of a glycoprotein (G); and (b) a non-Rhabdoviral nucleic acid. In other embodiments, the non-Rhabdoviral nucleic acid encodes for a cytokine or suicide gene.

In another embodiment, this invention provides a method for identifying an agent that has oncolytic activity, comprising the steps of: obtaining vibrotome slices of corona, substantia negra and cortex tissue, culturing said slices on coverslips under conditions maintaining viability and inhibiting mitosis, inoculating said slice culture with labeled cancer cells, culturing said inoculated culture with a candidate agent, and determining cancer cell viability, wherein a decrease in cancer cell viability indicates that the candidate agent is oncolytic, thereby identifying an agent that has oncolytic activity. In one embodiment, the cancerous cells are of neuronal origin. In another embodiment, the cancerous cells are labeled with a fluorescent, luminescent, chromogenic or electron dense material. In another embodiment, the method further comprises the step of inoculating the slice culture with labeled recombinant Rhabdovirus, and/or culturing the inoculated slice culture with a cytokine.

### BRIEF DESCRIPTION OF THE DRAWINGS

FIG. 1: is a schematic representation of the method used for isolating noncytopathic VSV mutants.

FIG. 2A-D: represents phase contrast images of cells infected with wild-type (wt) VSV (A); a temperature-sensitive mutant of VSV (tsO82) which contains mutations in the M gene (B); the M33;51A recombinant virus used to select for the NCP mutant (C); and one of the plaque-purified NCP variants (NCP-12) (D). FIG. 2a-d represent phase contrast images of cells infected with wild-type (wt) VSV (A); a temperature-sensitive mutant of VSV (tsO82) which contains mutations in the M gene (13); the M33;51A recombinant virus used to select for the NCP mutant (C); and one of the plaque-purified NCP variants (NCP-12) (D). Note that NCP-12 infected cells have grown to confluence and have a morphology indistinguishable from uninfected BHK cells (not shown).

FIG. 3: is a schematic representation of the methods used to clone and sequence one of the NCP mutants (NCP-12).

FIG. 4: is a schematic representation used to recover recombinant viruses encoding NCP variants.

FIG. 5A-E: represents immunofluorescence and phase contrast images of wt-VSV(A-B) and rVSV/M_{NCP12.1}(C-E) mutant infected BHK-21 cells. FIG. 5E is a magnification of the cell monolayer.

FIG. 6 represents expression level analysis of the M_{NCP12.1} mutant protein from a eukaryotic expression vector. BHK-21 cells were transiently transfected with 2 μgs of pCAGGS-M wt (left panel), pCAGGS-NCP-12.1 (two middle panels), or pCAGGS-MCS plasmids (right panel). Cells were analyzed using an M-specific monoclonal antibody (23H12) and a rhodamine conjugated goat anti-mouse secondary antibody.

FIG. 7A-H: represents phase contrast (A-D) and fluorescence analysis (E-H) of infection of different cell types by rVSV/M_{NCP12.1}.

FIG. 8: is a schematic representation of the method used to recover a prototypic VSV gene delivery/gene therapy vectors, which lacks M protein.

FIG. 9: represents the method and analysis of the recovery and passaging of rVSV-ΔM (VSV replicon): Analysis was performed using an N-specific monoclonal antibody and a rhodamine conjugated goat anti-mouse secondary antibody.

FIG. 10: represents fluorescence (top panels) and phase contrast (bottom panels) of islet cell sample 176 infection with VSV deleted for M, and deleted for G and M proteins, as indicated, at an MOI of 5.

FIG. 11: represents fluorescence (top panels) and phase contrast (bottom panels) of islet cell sample 163 infection with VSV deleted for M, and deleted for G and M proteins, as indicated, at an MOI of 5.

FIG. 12: represents fluorescence (top panels) and phase contrast (bottom panels) of islet cell sample 176 infection with an MOI of 25, 3 days post-infection.

FIG. 13: represents fluorescence (top panels) and phase contrast (bottom panels) micrographs of islet cell sample 163 infection with an MOI of 25, 3 days post-infection.

FIG. 14: represents fluorescence (top panels) and phase contrast (bottom panels) micrographs of islet cell sample 176 infection with an MOI of 5, 8 days post-infection.

FIG. 15: represents fluorescence (top panels) and phase contrast (bottom panels) micrographs of islet cell sample 176 infection with an MOI of 25, 8 days post-infection.

FIG. 16: represents fluorescence (top panels) and phase contrast (bottom panels) micrographs of islet cell sample 163 infection with an MOI of 5, 8 days post-infection.

FIG. 17: represents fluorescence (top panels) and phase contrast (bottom panels) micrographs of islet cell sample 163 infection with an MOI of 25, 8 days post-infection.

FIG. 18: represents fluorescence analysis of islet cell sample 176 at 3 days post-infection with an MOI of 5, or 25 (top and bottom panels, respectively).

FIG. 19: represents fluorescence analysis of islet cell sample 163 at 3 days post-infection with an MOI of 5, or 25 (top and bottom panels, respectively).

FIG. 20: represents a sequence alignment of the membrane-proximal domains of vesiculovirus glycoproteins. The sequences shown are from the San Juan (37) and Orsay strains (19) of VSV Indiana, VSV New Jersey (18), Cocal virus (2), Chandipura virus (28), Piry virus (4) and spring viremia of carp virus (SVCV) (3). Residues in black colored font with light gray background are conserved among all the vesiculoviruses. Residues in white font with black background are identical residues in the virus sequences examined. Residues in black font with dark gray background indicate residues with similar properties. Stars at the bottom of the sequence represent invariant residues across the sequences examined.

FIG. 21: schematic representation of mutations in the membrane-proximal “stem” region of VSV G. A linear diagram of the full-length G protein is shown at the top with the ectodomain, juxtamembrane G-stem (GS) region, transmembrane (TM) and cytoplasmic domains demarcated. The sequence of the 42 amino acid stem region is also shown. The numbers at the beginning and end of the sequence indicate the position of the amino acid residues from the N-terminus of VSV GIND (San Juan strain). Amino acid K462 is the boundary between the TM domain and ectodomain. Mutations in the conserved tryptophan (W) residues (positions 457 and 461), the glutamic acid (E452), the glycine (G456), and the phenylalanine (F458) are shown. The sequence of the insertion, deletion, and inverted sequence mutants are also shown. The protein G(AXB) introduces two additional serines at the ectodomain TM junction.

FIG. 22: demonstrates the expression and stability of the mutant proteins. COS-1 cells were transfected with plasmids encoding the indicated G proteins, the proteins were labeled with [³⁵S]-methionine and then analyzed by immunoprecipitation with a polyclonal anti-G antibody followed by SDS-PAGE. (A) Substitution mutants and wild-type G protein (WT-G). (B) Deletion and insertion mutants. The lanes labeled VSV are immunoprecipitated proteins from cells that were infected with wild-type VSV and labeled with [³⁵S]-methionine. The positions of the G and N proteins are indicated.

FIG. 23: represents the transport kinetics of wild type and mutant G proteins. BHK-21 cells expressing wild-type G or the mutant proteins were labeled with ³⁵S-Methionine for 15 minutes. The media was removed and medium containing excess unlabeled methionine was added for 0, 10, 30 or 60 minutes. The G proteins were immunoprecipitated from cell lysates using an anti-G tail peptide antibody. One half of the immunoprecipitates were digested with endoglycosidase H. Proteins were resolved on a 10% SDS-PAGE gel and visualized by fluorography. The amounts of Endo H resistant and sensitive forms of the proteins were quantified using ImageQuant software (Molecular Dynamics, Co). (A) Results of an experiment examining wild-type (WT); GΔ13, and Gsrev11. (B) Results from a separate experiment comparing WT, G10DAF, ΔF440-N449, and GΔ9-10DAF.

FIG. 24: represents WT and mutant virus infected cell syncytium formation. Approximately 5×10⁵ BHK-21 cells were infected at a multiplicity of 10 for 1 hour at 37° C. Six hours post-infection the cells were treated with fusion medium buffered to pH 5.9, 5.5, or 5.2 for 1 minute at room temperature. The media was replaced with DMEM+5% FBS and the cultures were incubated at 37° C. for 20 minutes to 1 hour. Cells were then fixed and processed for indirect immunofluorescence using a G-specific mAb (I1). Rhodamine conjugated goat anti-mouse antibody was used as the secondary Ab. Fluorescence and phase contrast images were digitally captured using a Zeiss Axiocam fitted on a Zeiss Axiophot microscope with a 10× water immersible ceramic objective. The images were then processed using Adobe Photoshop to adjust for brightness and contrast. (A) Syncytia formation induced in cells infected with rVSV-wt, -GΔ9, -GΔ13, and -GΔ9-10DAF after treatment with fusion media buffered to pH 5.9. The arrows point to small syncytia in the mutant infected cells. (B) Cells infected with rVSV-ΔF440-N449, -G10DAF and -G(+9)gBG after treatment at pH 5.2.

FIG. 25: represents WT and mutant virus infectivity. BHK-21 cells were infected with either WT or G-complemented mutant viruses at a multiplicity of 10 for 1 hr and then the cells were washed 3 times with growth medium. Sixteen hours post-infection an aliquot of the supernatant was taken and used to determine virus titers using plaque assays on BHK-21 cells. Thirty-six hours post-infection the number of plaques were counted and averaged between at least two dilutions to determine the titers. Virus titers shown are the average from at least three independent experiments.

FIG. 26: represents incorporation of WT and mutant G proteins in virions. BHK-21 cells were infected with viruses encoding the wild-type or mutant proteins at a multiplicity of 10 as described in the legend to FIG. 5. Sixteen hours post-infection virus released into the supernatant was pelleted through a 20% sucrose cushion. The viral pellets were resuspended in sample buffer and the proteins from one-fifth of the viral pellets were resolved by SDS-PAGE. The proteins were visualized by staining with Coomassie blue. Digital images of the gels were obtained using a Nikon camera with a 35-80 mm Nikkor lens. Protein amounts were quantified by densitometry using Image Quant software (Molecular Dynamics, Co.). Relative amounts of G protein incorporated into virions were determined by calculating the ratio of G protein to N protein. The results are expressed as a percentage relative to the G:N ratio found in the wild-type VSV control.

FIG. 27: represents WT and mutant viral binding. Radiolabelled virions (˜80,000 cpm) were resuspended in binding media buffered to pH 7.0 or 5.9 and incubated at room temperature for 30 minutes. The suspensions were then cooled on ice for 10 minutes and then added to pre-chilled confluent monolayers of BHK-21 cells. Virus binding was done for 3 hrs on ice. The medium was removed and the amount of radioactivity was determined. This represented the unbound virus fraction. The cells were then washed three times with ice-cold binding buffer at the same pH used for binding and the washes were collected for quantitation. Cells were lysed in PBS containing 1% TX-100 and the amount of radioactivity in the lysates (bound fraction) was determined. Virus binding was expressed as a percentage of bound virus to the total.

FIG. 28: represents the construction of recombinant replication-restricted VSV expressing an IL-12 fusion protein. A bioactive murine IL-12 fusion construct was produced by removing the stop codon of the p40 subunit, removing the first 22 codons on the p35 subunit, and inserting a sequence coding for a Gly-Ser linker region as diagramed in Panel A. A diagrammatic representation of the recombinant anti-genome of VSVΔG-IL12F is shown in Panel B. The anti-genome encodes the nucleocapsid (N), polymerase (P and L), and matrix (M) proteins of VSV. In addition, instead of encoding the envelope glycoprotein (G), the entire G coding region has been removed and replaced with a multiple cloning site (MCS). The cDNA encoding the IL-12 fusion construct was inserted into this MCS. To produce a precise 3′ end of the VSV antigenomic RNA, a ribozyme (RBZ) from hepatitis delta virus was placed immediately following the VSV trailer. This anti-genome RNA was expressed from a pBluescript background, and its transcription is driven from a T7 promoter.

FIG. 29: represents the production and secretion of v IL-12F in VSVΔG-IL12F infected cells. BHK-21 cells were infected with VSVΔG-IL12F (MOI=5) and were cultured in serum-free medium for 17 hours. Supernatants were harvested and BHK cells that had detached from the plate were removed by low-speed centrifugation (pre-clarified supernatant). Virions were removed from the pre-clarified supernatant by pelleting at 100,000×g over a 20% sucrose cushion (clarified supernatant). To assess production of vIL-12F, samples of pre-clarified and clarified supernatant, as well as pelleted virions were subjected to SDS-PAGE on a 10% gel. Resolved proteins were visualized by Coomassie blue staining (A). The sample compositions were as follows: lane 1) 100 μl pre-clarified supernatant, lane 2) 50 μl clarified supernatant, lane 3) 100 μl clarified supernatant, and lane 4) virus pelleted from 500 μl supernatant. In parallel, a similar gel was transferred to nitrocellulose for Western blotting with an IL-12-p40-specific monoclonal antibody preparation (B). The sample compositions were as follows: lane 1) 100 μl pre-clarified supernatant, lane 2) 100 μl clarified supernatant, and lane 3) virus pelleted from 500 μl supernatant. The identity of each virally-expressed protein is indicated.

FIG. 30: represents vIL-12F potentiation of antigen-specific T cell responses to listerial antigens. C3HeB/FeJ mice (5/group) were immunized on days 0, 5, and 15 with either PBS (vehicle), LMAg (109 HKLM+8 μg soluble Listeria protein)+PBS, LMAg+0.5 μg rIL-12, LMAg+0.5 μg vIL-12F, or LMAg+5.0 μg vIL-12F. On day 20, mice were sacrificed and peritoneal exudate cells were collected by lavage, pooled, and plastic non-adherent cell populations (PNA) were prepared. PNA (1.5×106/ml) were restimulated in vitro (24 h at 37° C.) with pre-determined optimal concentrations of either culture medium (no stimulation), Con A (2 μg/ml; polyclonal stimulator), HKLM (107/ml), or SLP (8 μg/ml). IL-2 (A) and IFN-□ (B) in cell free supernatants were quantitated as a measure of antigen-specific T cell responsiveness. All assays were performed in triplicate, and results are expressed as mean±SD.

FIG. 31: represents the induction of distinct resident cell population profiles upon co-administration of listerial antigen and vIL-12F. C3HeB/FeJ mice (5/group) were immunized on days 0, 5, and 15 with either PBS (vehicle) LMAg (109 HKLM+8 μg soluble Listeria protein)+PBS, LMAg+0.5 μg rIL-12, LMAg+0.5 μg vIL-12F, or LMAg+5.0 μg vIL-12F. On day 20, mice were sacrificed and peritoneal exudate cells were collected by lavage, pooled, and plastic non-adherent cell populations (PNA) were prepared. PNA (5×105/ml) were stained with the indicated fluorochrome-conjugated antibody preparations; staining with isotype control antibody preparations was also performed to assess non-specific antibody binding. Flow cytometric analysis of the lymphocyte population of each sample (selected by forward scatter/side scatter gating) was performed. (A) Cells were double-stained with anti-CD5 PE and anti-CD45R/B220 FITC (or rat IgG2a PE and rat IgG2a FITC as isotype controls), and flow cytometric analysis was performed. The frequency of T cells within the lymphocyte population is indicated for each test panel. (B) Cells were doubled-stained with anti-CD3 PE and either anti-αβ TCR FITC, anti-γδ TCR FITC, anti-CD4 FITC, or anti-CD8 FITC and flow cytometric analysis was performed. To characterize TCR or CD4/CD8 expression on CD3+ cells, samples were further gated on the CD3+ population. The frequency of the indicated cell types within the lymphocyte populations are shown in graphical format as follows: (C) T cells, conventional B cells, and B1 B cells, (D) CD3+ cells. The frequencies of αβ and γδ TCR expression (E) as well as CD4 and CD8 expression (F) within the CD3+ population are also shown in graphical form.

FIG. 32: represents the eliciting of protective listerial immunity following co-administration of listerial antigen and vIL-12F. C3HeB/FeJ mice (5/group) were immunized on days 0, 5, and 15 with either PBS (vehicle), LMAg (109 HKLM+8 μg soluble Listeria protein)+PBS, 5.0 μg vIL-12F+PBS, LMAg+0.5 μg vIL-12F, or LMAg+5.0 μg vIL-12F. An additional group of 5 mice was inoculated i.p. with a sublethal dose of viable Listeria on day 0 (6×103/mouse or 0.12×LD₅₀). On day 45, each mouse received (i.p.) a challenge dose of viable Listeria (6.4×105 or 12.9×LD₅₀). Mice were killed 4 days later (day 49) and bacterial load in the spleen (A) and liver (B) of each mouse was quantitated.

FIG. 33: represents the long-lived protective immunity conferred by immunization with listerial antigen and IL-12F. C3HeB/FeJ mice (5/group) were immunized on days 0, 5, and 15 with either PBS (vehicle), 5.0 μg vIL-12F+PBS, LMAg (109 HKLM+8 μg soluble Listeria protein)+PBS, or LMAg+5.0 μg vIL-12F. An additional group of 5 mice was inoculated i.p. with a sublethal dose of viable Listeria on day 0 (6×10³/mouse or 0.12×LD₅₀). On day 120, each mouse received (i.p.) a challenge dose of viable Listeria (3.8×10⁵ or 7.6×LD₅₀). Mice were killed 4 days later (day 124) and bacterial load in the spleen (A) and liver (B) of each mouse was quantitated.

FIG. 34: represents VSV-wt infection of C6 gliomas. C6-GFP cells grown in a 6-well dish were infected with 10⁵ pfu of rVSV-DsRed. Phase contrast images of cells at A) time zero (B) 10 hours post-infection, (C) 24 hours post infection and (D) 48 hours post infection are shown. Images were collected using a Zeiss Axiocam digital camera mounted on a Zeiss Axioskop microscope with a 10× objective.

FIG. 35: represents the cytotoxicity of rVSV-DsRed for C6-GFP glioma cells. C6-GFP glioma cells were plated at 90% confluency in 96 well plates and infected with 10, 1,000 or 10,000 pfu of rVSV-DsRed. Cultures were monitored for cell viability using the CellTiter MTS assay for up to 96 hours. Cell viability was reduced to 50% by 30 hours post-infection and by 72 hours little to no metabolic activity was detected, irrespective of the dose of virus used.

FIG. 36: represents a rat organotypic brain slice culture and brain slice-C6-GFP glioma coculture. The culture was established using three areas of rat brain (substantia nigra, striatum, and cortex) as shown in (A). TH and MAP-2 immunoreactivity of slices after 2 weeks of culture are shown in panels B and C, respectively. (D) shows a low magnification (4×) photomicrograph of C6-GFP cells and (E) shows a higher magnification (40×) image of the cells using a fluorescence microscope.

FIG. 37: represents rVSV-DsRed infection of normal and slice-glioma coculture over a three day incubation period. (A) Infection of normal slice tissues after inoculation with 10⁴ pfu of rVSV-DsRed. (B) Infection of normal slice tissues with 104 pfu rVSV-DsRed after pre-incubation of the slice culture with IFN-β. (C) Destruction of C6-GFP glioma cells by inoculation with 10⁴ pfu rVSV-DsRed after pre-incubation of slice-glioma coculture with IFN-β. (D) Visualization of red fluorescence (e.g. rVSV-DsRed infection) in slice tissues after pre-incubation of slice-glioma coculture with IFN-β followed by inoculation with 10⁴ pfu rVSV-DsRed. These data demonstrate that wild-type VSV infection of normal slice tissues is significantly blocked by IFN-β, and that rVSV-DsRed can effectively destroy C6-GFP glioma growing in the slice culture.

FIG. 38: represents MAP-2 immunoreactivity of slices inoculated with rVSV-DsRed at three days post-infection. (A) MAP-2 immunoreactivity of normal slice culture at 3 days post-infection with rVSV-DsRed. (B) MAP-2 immunoreactivity of normal slice culture 3 days post-infection after incubation with 1,000 U IFN-β24 hours prior to inoculation with rVSV-DsRed. (C) MAP-2 immunoreactivity of slice-glioma coculture after pre-treatment with IFN-β followed by infection with rVSV-DsRed. Red staining identifies infected cells, green staining identifies C6-GFP cells, and blue represents MAP-2 staining. (D) MAP-2 immunoreactivity of normal slice cultures after incubation with IFN-β for three days. These data indicate that pre-incubation with IFN-β can reduce toxic effects seen with rVSV-DsRed alone, but not in the slice-glioma coculture. These data also show that IFN-β alone does not appear to be directly toxic to the slice tissues.

FIG. 39: represents rVSV-ΔG infection of normal and slice-glioma coculture over a three day incubation period. (A) Time course of viral replication in normal slice tissues after inoculation with 10⁶ infectious units of Infectious ΔG-DsRed as shown by expression of Ds-Red (e.g. red fluorescence). (B) Pre-incubation of the slice culture with IFN-β prevents infection of normal cells, following inoculation with 10⁶ infectious units of ΔG-DsRed. (C) Destruction of C6-GFP glioma by inoculation with 10⁶ infectious units of ΔG-DsRed following pretreatment with 1,000 U IFN-β. (D) Pretreatment of the slice-glioma coculture with IFN-β prevents infection of normal cells following inoculation with 10⁶ infectious units of ΔG-DsRed. These data demonstrate that ΔG-DsRed infection of normal slice tissues is significantly blocked by IFN-β, and that ΔG-DsRed can effectively destroy C6-GFP glioma growing in the slice culture.

FIG. 40: represents MAP-2 immunoreactivity in normal slice cultures and slice-glioma cocultures following inoculation with ΔG-DsRed. (A) MAP-2 immunoreactivity of normal slice cultures 3 days post-inoculation with ΔG-DsRed. (B) MAP-2 immunoreactivity of normal slice cultures pre-treated with 1,000 U IFN-β followed by inoculation with ΔG-DsRed. (C) MAP-2 immunoreactivity of slice-glioma cocultures after pre-treatment with IFN-β followed by innoculation with ΔG-DsRed. These data indicate that VSV-ΔG is less toxic than VSV-wt and, that overall toxicity of VSV-ΔG is reduced with pre-incubation with IFN-β.

FIG. 41: represents photomicrographs of an in vivo rat brain tumor model. Rats are injected with C6-GFP tumor cells and sacrificed at two weeks. A. H&E staining of a rat brain coronal frozen section demonstrating a large tumor with central necrosis in the right hemisphere. B. An adjacent section visualized for GFP expression, using fluorescence microscopy, GFP fluorescent tumor cells at (C) 4× and (D) 10× respectively.

FIG. 42: represents photomicrographs of ITGA-3 (611045, BD Transduction Laboratories) immunoreactivity of frozen sections taken from the center (A) and periphery (B) of an in vivo rat C6 glioma. GFP C6 cells were counterstained with glioma marker ITGA-3 in red. As expected, tumor cells were shown to be immunoreactive (10×, 40×, respectively) using fluorescence confocal microscopy.

### DETAILED DESCRIPTION OF THE INVENTION

The present invention provides recombinant viruses, recombinant Rhabdoviridae, vectors and compositions comprising same. In one embodiment, Rhabdoviral nucleic acid sequences of the invention comprise matrix proteins (M) and/or glycoproteins (G) that are mutated or partially deleted and therefore can be used for the production of Rhabdovirus-based gene therapy vectors, vaccines and/or anti-cancer therapies. The invention provides, in other embodiments, methods of producing and therapeutic applications of the recombinant Rhabdoviridae, vectors and compositions herein disclosed.

Recombinant Rhabdoviridae provide, in one embodiment, a means of foreign gene delivery that is highly versatile, since they infect many different cell types in the human body. Their manipulation to express heterologous proteins provides, in another embodiment, a system for foreign gene delivery to a wide array of cell types, an application that has been lacking in many previous vectors used for gene delivery, with a much narrower cellular tropism.

Previous use of recombinant Rhabdoviridae resulted in cytopathic effects, with minimal foreign protein expression, owing to depressed cellular protein synthesis, a by-product of Rhabdoviral infection. In the present invention however, in one embodiment, the recombinant Rhabdoviridae, mutated or deleted for the M protein, infect cells, yet are not cytopathic (Example 1). Heterologous protein expression was readily accomplished, with enhanced protein expression in mutated viruses.

Mutation of the M protein did not alter cellular tropism, hence multiple cell types were infected (Example 2), and were readily recoverable (Example 3). Islet cell infection with the mutated virus resulted in high levels of foreign gene expression, with minimal cytopathic effects (Example 4). Further, infection with strains deleted for both M and G glycoprotein resulted in high levels of infection, and expression, even after 8 days in culture. Thus recombinant Rhabdoviridae mutated or deleted for the M protein, with or without concurent mutation or deletion of the G protein, provided a gene delivery/gene therapy vector, which was non-cytopathic.

In one embodiment, there is provided a recombinant Rhabdovirus comprising a nucleic acid of a Rhabdoviral genome wherein said Rhabdoviral genome comprises a deletion or a mutation within a region encoding a matrix protein (M). According to this aspect of the invention, in one embodiment, the recombinant Rhabdovirus is non-cytopathic.

As used herein, the terms “recombinant Rhabdovirus” and “recombinant Rhabdoviridae” refer to virus genetically engineered to express proteins not natively expressed in Rhabdoviridae. Engineering of the virus in this manner therefore creates a “pseudotype” or “chimeric” virus that can subsequently be isolated.

As used herein, the term “Non-cytopathic Rhabdovirus” means non-cytopathic variants of Rhabdovirus that still function in viral assembly but are not cytopathic to infected cells.

As used herein, the term “matrix protein (M)” refers to a protein encoded in the Rhabdovirus genome. The matrix protein lies within the membrane envelope, perhaps interacting both with the membrane and the nucleocapsid core. The matrix protein of Rhabdovirus serves two critical functions in the life cycle of the virus. First, it is essential for virus assembly and the release of virus particles from infected cells. Second, it is responsible for the inhibition of host cell gene expression, which allows the virus to utilize all of the host cell translation machinery for the synthesis of viral proteins. The inhibition of host gene expression by M protein is thought to be responsible for the severe and rapid cytopathic effects associated with a Rhabdovirus infections.

In one embodiment, the recombinant non-cytopathic Rhabdovirus of the invention comprises a mutation or a deletion in the matrix protein M. In another embodiment, the mutation is in a region encoding the N-terminal half of the matrix protein, which may comprise the region encoding a nuclear localization signal (NLS).

As used herein, the term “nuclear localization sequence” or “NLS” refers to a peptide, or derivative thereof, that directs the transport of an expressed peptide, protein, or molecule associated with the NLS; from the cytoplasm into the nucleus of the cell across the nuclear membrane.

In one embodiment, the mutation encodes for an alanine residue instead of a methionine residue, such as, for example at position 33 or 51 of the matrix protein (M). In another embodiment, the mutation encodes for the substitution of a glycine residue for a serine residue, which may be, for example, at position 226. In another embodiment, the mutation encodes for the substitution of an alanine residue for a threonine residue, such as, for exmaple, at position 133. The mutation may also comprise a deletion in the entire M protein coding region, in another embodiment. Any alteration in M protein expression, resulting in diminished cytopathic effects of Rhabdoviridae is to be considered as part of the present invention.

In another embodiment, an M protein mutant has an amino acid sequence that corresponds to SEQ ID NO: 1, 2, 3, 4 or 5.

In one embodiment of this invention, the recombinant non-cytopathic Rhabdovirus may further comprise a mutation within the region encoding a glycoprotein (G).

The glycoprotein (G) encoded by Rhabdoviridae contributes to viral fusion, infectivity and the overall efficiency of the viral budding process (Whitt M. A., (1998) The Journal of Microbiology 36: 1-8). A fragment of the Rhabdoviral G protein, the G stem polypeptide, is involved in membrane fusion. As used herein, the phrase “G stem polypeptide” refers to segments of the Rhabdoviral G protein, comprising a 42 amino acid membrane-proximal ectodomain, a transmembrane anchor domain and a cytoplasmic tail domain of the mature G protein.

Since the G glycoprotein is involved in membrane fusion, it facilitates cell-to-cell spread in Rhabdoviral infection. The membrane-proximal ectodomain of G was shown herein to be essential for membrane fusion (Examples 6-7). Substitution, deletion or insertion mutations of the region encoding the membrane-proximal ectodomain of G did not result in diminished G expression (Example 5). While none of mutations in the membrane-proximal region affected stability, oligomerization or transport of the full-length G proteins to the cell surface, deletions in the region resulted in profoundly suppressed fusion, as did the insertion of 9 or 10 amino acids between the boundary of the membrane anchoring domain and the G protein. Substitution of specific residues from the membrane-proximal ectodomain of G however, did not diminish fusion. Only deletion of the region between F440 and N449 which includes the conserved FFGDTG motif completely abolished fusion activity showing that this sub-domain is important for the fusion activity of G. The fusion profile accompanied the viral growth profile, with deletion mutants requiring complementation with a functional G for promoting viral growth (Example 8). Deletion of membrane proximal ectodomain amino acid residues, for example N449-K462 result in diminished infectivity as well.

In one embodiment of this invention, there is provided a recombinant Rhabdovirus comprising a nucleic acid of a Rhabdoviral genome wherein the Rhabdoviral genome comprises a deletion or a mutation within a region encoding a membrane-proximal ectodomain of a Rhabdoviral glycoprotein (G).

In one embodiment, the mutation in the region encoding a membrane-proximal ectodomain of a Rhabdoviral glycoprotein (G) encodes for the substitution of an alanine amino acid residue for a tryptophan amino acid residue (SEQ ID NO: 8, 10, 11, 12, 13 or 14). In another embodiment, the mutation encodes for the substitution of an alanine amino acid residue for a glutamic acid (SEQ ID NO: 6), glycine (SEQ ID NO: 7) and/or phenylalanine amino acid residue (SEQ ID NO: 9). In another embodiment, the mutation encodes for the substitution of aspartic acid and alanine amino acid residues instead of a glutamic acid, glycine and/or phenylalanine amino acid residue. In another embodiment, the mutation is any combination of the mutations encoding for the amino acid residue replacements listed herein.

In another embodiment, the mutation in the region encoding a membrane-proximal ectodomain of a Rhabdoviral glycoprotein (G) encodes for the deletion of nucleotides in the ectodomain. In one embodiment, the mutation is a deletion of the nucleotides encoding for amino acid residues 449-461 (SEQ ID NO: 20), or a fragment thereof of the Rhabdoviral G glycoprotein. In another embodiment, the mutation is a deletion of the nucleotides encoding for amino acid residues 440-449 (SEQ ID NO: 16), or a fragment thereof.

In another embodiment, the mutation in the region encoding a membrane-proximal ectodomain of a Rhabdoviral glycoprotein (G) encodes for the insertion of nucleotides in the ectodomain. In one embodiment, the mutation is an insertion of the nucleotides encoding for the amino acid residues 311-319 of decay acceleration factor (DAF) inserted between serine amino acid residues of the Rhabdoviral glycoprotein membrane proximal ectodomain (SEQ ID NO: 22).

In another embodiment, a mutation in the coding region for membrane-proximal ectodomain of a Rhabdoviral glycoprotein results in a mutant with an amino acid sequence corresponding to SEQ ID NO: 15, 19, 20 or 22.

The Rhabdoviral genome may further comprises a mutation or deletion in a matrix protein (M), in another embodiment.

It is to be understood that mutations in the membrane-proximal ectodomain of the Rhabdoviral G protein, as in the Rhabdoviral M protein, may result in partial deletions, or complete deletion of the G membrane-proximal ectodomain/M protein-coding region, and are to be considered as part of this invention. Similarly, insertional mutations within the G membrane-proximal ectodomain/M protein coding region are envisaged as part of this invention. Mutations resulting in loss of function, or altered expression of the Rhabdoviral G membrane-proximal ectodomain/M protein are contemplated herein as well, and comprise additional embodiments of the present invention.

In one embodiment, the recombinant Rhabdovirus utilized for this invention is derived from Vesicular Stomatitis Virus (VSV), though the invention provides for the utilization of any virus of the Vesiculovirus and Lysavirus genus. The Vesiculovirus genus includes: Vesicular Stomatitis Virus (VSV) of the New Jersey serotype (VSVNJ), the Indiana serotype (VSVInd), the VSV-Alagoas strain, Cocal virus, Jurona virus, Carajas virus, Maraba virus, Piry virus, Calchaquivirus, Yug Bogdanovac virus, Isfahan virus, Chandipura virus, Perinet virus, and Porton-Svirus (Rose and Whitt. IN B. N. FIELDS' VIROLOGY 4th ED. VOL. 1 (2001)). The Lyssavirus genus includes: Rabies virus (RV), Lagos bat virus, Mokola virus, Duvenhagevinus, Obodhiang virus, and Kotonkan virus (ID.)

In another embodiment, this invention provides recombinant Rhabdoviridae as described hereinabove, further comprising a nucleic acid sequence encoding a heterologous fusion facilitating polypeptide. In another embodiment of the present invention, the nucleic acid sequence encoding for a fusion facilitating polypeptide may be expressed from a separate transcriptional unit.

As used herein, the term “fusion-facilitating polypeptide” refers to any protein (or fusion-facilitating polypeptide fragment thereof) that following expression on the surface of a vesicular membrane precipitates fusion of the vesicular membrane with a lipid-bilayer encasing a target vesicle or cell. In another embodiment of the present invention, the fusion-facilitating polypeptide is: (1) is derived from a virus characterized as having a lipid envelope; and (2) when expressed as a heterologous protein in a genetically engineered virus, facilitates the fusion of the viral envelope with a cell membrane, resulting in a complete bilayer fusion between participating membranes. It is thus envisioned that a fusion-facilitating polypeptide according to the present invention can function in a non-specific fashion in facilitating the association of an attachment protein on the viral envelope other than the native viral attachment protein. One example of a fusion-facilitating polypeptide as contemplated herein is the viral envelope fusion protein known in the literature as the “F protein” of the SV5 strain of Paramyxoviruses, which specifically is referred to herein as the “F protein” rather than the more generic “Fusion Protein”.

In addition to simian virus 5 (SV5)-derived F proteins, fusion-facilitating polypeptides may be selected from HIV envelope proteins, as well as VSV G_{NJ} (New Jersy serotype) or VSV G_{IND} (Indiana serotype) proteins. Also included are polypeptides exhibiting at least 70% amino acid sequence homology to the above mentioned fusion polypeptides, as well as polypeptides exhibiting significant functional homology in terms of stimulating target cell fusion with the recombinant Rhabdoviridae and expressed nucleic acid sequences of the present invention. It is to be understood that utilization of any protein stimulating membrane fusion, or a fragment thereof is to be considered within the scope of the invention, as are homologues of such proteins and their fragments, and that these proteins may be of prokaryotic or eukaryotic origin. Proteins and polypeptides derived by protein evolution techniques well known to those skilled in the art are envisaged as well, and represent additional embodiments of the invention.

The recombinant Rhabdoviridae may, in one embodiment, further express at least one heterologous (i.e, another non-Rhabdoviral) protein.

In another embodiment, the recombinant Rhabdoviridae of this invention may further comprise a regulatory element.

Nucleotide sequences which regulate expression of a gene product (which are referred to herein as “regulatory elements”, for example, promoter and enhancer sequences) are selected, in one embodiment, based upon the type of cell in which the gene product is to be expressed, or in another embodiment, upon the desired level of expression of the gene product, in cells infected with the recombinant Rhabdoviridae of the invention. According to this aspect of the invention, the gene product corresponds to the heterologous protein, as described herein. Regulated expression of such a heterologous protein may thus be accomplished, in one embodiment.

For example, a promoter known to confer cell-type specific expression of a gene linked to the promoter can be used. A promoter specific for myoblast gene expression can be linked to a gene of interest to confer muscle-specific expression of that gene product. Muscle-specific regulatory elements which are known in the art include upstream regions from the dystrophin gene (Klamut et al., (1989) Mol. Cell Biol. 9:2396), the creatine kinase gene (Buskin and Hauschka, (1989) Mol. Cell Biol. 9:2627) and the troponin gene (Mar and Ordahl, (1988) Proc. Natl. Acad. Sci. USA. 85:6404).

Regulatory elements specific for other cell types are known in the art (e.g., the albumin enhancer for liver-specific expression; insulin regulatory elements for pancreatic islet cell-specific expression; various neural cell-specific regulatory elements, including neural dystrophin, neural enolase and A4 amyloid promoters). In another embodiment, a regulatory element, which can direct constitutive expression of a gene in a variety of different cell types, such as a viral regulatory element, can be used. Examples of viral promoters commonly used to drive gene expression include those derived from polyoma virus, Adenovirus 2, cytomegalovirus and Simian Virus 40, and retroviral LTRs.

In another embodiment, a regulatory element, which provides inducible expression of a gene linked thereto can be used. The use of an inducible regulatory element (e.g., an inducible promoeter) allows for modulation of the production of the gene product in the cell. Examples of potentially useful inducible regulatory systems for use in eukaryotic cells include hormone-regulated elements (e.g., see Mader, S. and White, J. H. (1993) Proc. Natl. Acad. Sci. USA 90:5603-5607), synthetic ligand-regulated elements (see, e.g., Spencer, D. M. et al 1993) Science 262:1019-1024) and ionizing radiation-regulated elements (e.g., see Manome, Y. Et al. (1993) Biochemistry 32:10607-10613; Datta, R. et al. (1992) Proc. Natl. Acad. Sci. USA 89:1014-10153). Additional tissue-specific or inducible regulatory systems, may be developed for use in accordance with the invention.

In one embodiment, the heterologous protein may be used as a therapeutic protein. By the term “therapeutic”, it is meant that the expression of the heterologous protein, when expressed in a subject in need, provides a beneficial effect. In some cases, the protein is therapeutic in that it functions to replace a lack of expression or lack of appropriate expression of such a protein in a subject. Some examples include cases where the expression of the protein is absent, such as in cases of an endogenous null mutant being compensated for by expression of the foreign protein. In other embodiments, the endogenous protein is mutated, and produces a non-functional protein, compensated for by the expression of a heterologous functional protein. In other embodiments, expression of a heterologous protein is additive to low endogenous levels, resulting in cumulative enhanced expression of a given protein.

In one embodiment, the therapeutic protein expressed may include cytokines, such as interferons or interleukins, or their receptors. Lack of expression of cytokines has been implicated in susceptibility to diseases, and enhanced expression may lead to resistance to a number of infections. Expression patterns of cytokines may be altered to produce a beneficial effect, such as for example, a biasing of the immune response toward a Th1 type expression pattern, or a Th2 pattern in infection, or in autoimmune disease, wherein altered expression patterns may prove beneficial to the host.

Recombinant VSV deleted for the glycoprotein (G) was engineered to express and secrete single-chain IL-12F, which produces large quantities of the cytokine (Example 9). Co-administeration of the VSVΔG-IL-12F with listerial antigens produced powerful Listeria-specific T cell-mediated immune responses that conferred long-lived, protective listerial immunity similar to that observed in mice immunized with LMAg+rIL-12 (Examples 10 & 11).

In one embodiment, there is provided a recombinant Rhabdovirus deleted for a G glycoprotein, engineered to express a cytokine. The cytokine may be an interleukin or interferon or a chemoattractant. In one embodiment, the cytokine is interleukin 2, interleukin 4, interleukin 12 or interferon-γ.

In another embodiment, the recombinant Rhabdovirus engineered to express a cytokine is mutated or deleted for the matrix protein. In another embodiment, the recombinant Rhabdovirus engineered to express a cytokine is mutated or deleted for the membrane-proximal ectodomain of the glycoprotein (G). It is to be understood that any recombinant Rhabdovirus of this invention may be further engineered to express a cytokine, and is to be considered as part of this invention.

In another embodiment, the therapeutic protein expressed may include an enzyme, such as one involved in glycogen storage or breakdown. In another embodiment, the therapeutic protein expressed may include a transporter, such as an ion transporter, for example CFTR, or a glucose transporter, or other transporters whose deficiency, or inappropriate expression results in a variety of diseases.

In another embodiment, the therapeutic protein expressed may include a receptor, such as one involved in signal transduction within a cell. Some examples include as above, cytokine receptors, leptin receptors, transferring receptors, etc., or any receptor wherein its lack of expression, or altered expression results in inappropriate or inadequate signal transduction in a cell.

In another embodiment, the therapeutic protein expressed may include a tumor suppressor gene, or a proapoptotic gene, whose expression alters progression of intracellular cancer-related events. For example, p53 may be expressed in cells that demonstrate early neoplastic events, thereby suppressing cancer progression.

In another embodiment, the therapeutic protein expressed may be selected from the group consisting of natural or non-natural insulins, amylases, proteases, lipases, kinases, phosphatases, glycosyl transferases, trypsinogen, chymotrypsinogen, carboxypeptidases, hormones, ribonucleases, deoxyribonucleases, triacylglycerol lipase, phospholipase A2, elastases, amylases, blood clotting factors, UDP glucuronyl transferases, ornithine transcarbamoylases, cytochrome p450 enzymes, adenosine deaminases, serum thymic factors, thymic humoral factors, thymopoietins, growth hormones, somatomedins, costimulatory factors, antibodies, colony stimulating factors, erythropoietin, epidermal growth factors, hepatic erythropoietic factors (hepatopoietin), liver-cell growth factors, interleukins, interferons, negative growth factors, fibroblast growth factors, transforming growth factors of the α family, transforming growth factors of the β family, gastrins, secretins, cholecystokinins, somatostatins, serotonins, substance P and transcription factors.

In another embodiment, the recombinant Rhabdoviridae contemplated by this invention further comprises an insertion of a heterologous nucleic acid sequence encoding a marker polypeptide. The marker polypeptide may comprise, for example, green fluorescent protein (GFP), DS-Red (red fluorescent protein), secreted alkaline phosphatase (SEAP), beta-galactosidase, luciferase, or any number of other reporter proteins known to one skilled in the art.

It may be desirable to specifically target a therapeutic protein to a particular cell. In another embodiment, in addition to expression of a therapeutic protein, a targeting protein is expressed, such that the recombinant Rhabdoviridae of the invention are directed to specific sites, where expression of therapeutic proteins occurs.

In one embodiment, recombinant Rhabdoviridae described herein are targeted to tumor cells, expressing, for example, the surface marker erbB. Such erbB⁺ cells, in turn, would be referred to herein as “target cells” as these cells are the population with which the recombinant Rhabdoviridae will ultimately fuse. Target cells often express a surface marker (referred to herein as “target antigen”) that may be utilized for directing the recombinant Rhabdoviridae to the cell, as opposed to neighboring cells, that are not tumor cells in origin and hence do not express erbB

The target antigen may be a receptor, therefore an “antireceptor,” also referred to as “attachment protein,” signifies a protein displayed on a recombinant Rhabdoviral envelope, or cell surface as described above, responsible for attachment of the viral particle/modified cell to its corresponding “receptor” on the target cell membrane. For example, the native antireceptor of the paramyxovirus SV5 is the viral NH protein; which binds sialic acid on host cell membranes. Fusion thus accomplished is mediated via the binding of an attachment protein (or “antireceptor”) on the viral envelope to a cognate receptor on the cell membrane.

As used herein, the term “attachment” refers to the act of antireceptor (expressed on viral particle lipid envelopes or engineered cell sufaces) recognition and binding to a target cell surface “receptor” during infection. The skilled artisan will recognize that attachment occurs prior to fusion of the attaching membrane with the target cell plasma membrane.

In another embodiment, recombinant Rhabdoviridae of the present invention express anti-receptors which function to direct the recombinant to virally infected cells, via anti-receptor binding to viral proteins expressed on infected cell surfaces. In this case, antireceptors to promote recombinant Rhabdoviridae fusion with virally-infected cells, will recognize and bind to virally expressed surface proteins. For example, HIV-1 infected cells may express HIV-associated proteins, such as gp120, and therefore expression of CD4 by recombinant Rhabdoviridae promotes targeting to HIV infected cells, via CD4-gp120 interaction.

The anti-receptor proteins or polypeptide fragments thereof may be designed to enhance fusion with cells infected with members of the following viral families: Arenaviridae, Bunyaviridae, Coronaviridae, Filoviridae, Flaviviridae, Herpesviridae, Hepadnaviridae, Orthomyxoviridae, Paramyxoviridae, Poxyiridae, Retroviridae, and Rhabdoviridae. Additional viral targeting agents may be derived from the following: African Swine Fever Virus, Borna Disease Virus, Hepatitis X, HIV-1, Human T Lymphocyte virus type-I (HTLV-1), HTLV-2, 1 5 lentiviruses, Epstein-Barr Virus, papilloma viruses, herpes simplex viruses, hepatitis B and hepatitis C.

In another embodiment, targeting virally-infected cells may be accomplished through the additional expression of viral co-receptors on the recombinant Rhabdoviridae/recombinant virus envelope, for enhanced fusion facilitation with infected cells. In one embodiment, the recombinant Rhabdoviridae/recombinant viruses are engineered to further express an HIV co-receptor such as CXCR4 or CCR5, for example.

Bacterial proteins expressed during intracellular infection are also potential targets contemplated for therapeutic intervention by recombinant Rhabdoviridae/recombinant viruses of the present invention. The intracellular bacteria may include, amongst others: Shigella, Salmonella, Legionella, Streptococci, Mycobacteria, Francisella and Chlamydiae (See G. L. Mandell, “Introduction to Bacterial Disease” IN CECIL TEXTBOOK OF MEDICINE, (W.B. Saunders Co., 1996) 1556-7). These bacteria would be expected to express a bacteria-related protein on the surface of the infected cell to which the recombinant Rhabdoviridae/recombinant viruses would attach.

In another embodiment, the targeting moieties may include integrins or class II molecules of the MHC, which may be upregulated on infected cells such as professional antigen presenting cells.

Proteins of parasitic agents, which reside intracellularly, also are targets contemplated for infection by the recombinant Rhabdoviridae/recombinant viruses. The intracellular parasites contemplated include for example, Protozoa. Protozoa, which infect cells, include: parasites of the genus Plasmodium (e.g., Plasmodium falciparum, P. Vivax, P. ovale and P. malariae), Trypanosoma, Toxoplasma, Leishmania, and Cryptosporidium.

Diseased and/or abnormal cells may be targeted using the recombinant Rhabdoviridae of the invention by the methods described above. The diseased or abnormal cells contemplated include: infected cells, neoplastic cells, pre-neoplastic cells, inflammatory foci, benign tumors or polyps, cafe au lait spots, leukoplakia, and other skin moles.

The recombinant Rhabdoviridae of the invention may be targeted using an anti-receptor that will recognize and bind to its cognate receptor or ligand expressed on the diseased or abnormal cell.

In another embodiment, diseased and/or abnormal cells may be uniquely susceptible to recombinant Rhabdoviral entry and cell lysis, as a result. In one embodiment, non-cytopathic recombinant Rhabdoviridae are cytopathic to diseased and/or abnormal cells alone. In one embodiment, the non-cytopathic recombinant Rhabdoviridae are further engineered to express a heterologous protein. In one embodiment, the heterologous protein may comprise all of the embodiments listed hereinabove. In another embodiment, the non-cytopathic recombinant Rhabdoviridae may comprise all of the embodiments listed herein, including further attenuation such as the incorporation of concurrent deletions in Rhabdoviral glycoprotein expression, or fragments thereof, such as the membrane proximal ectodomain of G.

Similarly, cells may be engineered to express Rhabdoviral genome components, by methods well known in the art. Nucleic acid vectors comprising the deleted or mutated Rhabdoviral M protein, further comprising, in one embodiment, deletions in the membrane-proximal ectodomain of G, or, in another embodiment, further deleted for G.

In another embodiment, the recombinant Rhabdoviridae of this invention may be engineered to express an antibody or polypeptide fragment thereof, a bi-functional antibody, Fab, Fc, Fv, or single chain Fv (scFv) as their attachment protein. Such antibody fragments may be constructed to identify and bind to a specific receptor. These antibodies can be humanized, human, or chimeric antibodies (for discussion and additional references see S. L. Morrison “Antibody Molecules, Genetic Engineering of,” in MOLECULAR BIOLOGY AND BIOTECHNOLOGY: A COMPREHENSIVE DESK REFERENCE 1995; S. D. Gillies et aL, (1990) Hum. Antibod. Hybridomas 1 (1): 47-54; E. HARLOW AND D. LANE, ANTIBODIES: A LABORATORY MANUAL (1988) Cold Spring Harbor Press, NY). Expression of functional single chain antibodies on the surface of viruses has been reported using Vaccinia virus (M. C. Galmiche et al., (1997) J. Gen. Virol. 78: 3019-3027). Similar methods would be utilized in creating a recombinant Rhabdovirus expressing a fusion facilitating protein and an antibody or antibody fragment. The genes encoding monoclonal antibodies that target, for example, tumor associated antigens (TAAs) expressed on a cell surface (e.g., prostate specific antigen (PSA)), can be isolated and used to produce the desired recombinant Rhabdovirus, or subcloned into an appropriate expression vector and expressed on a cell surface, as described above, through methodology well known to an individual skilled in the art.

Examples of antibodies include those antibodies, which react with malignant prostatic epithelium but not with benign prostate tissue (e.g., ATCC No. HB-9119; ATCC “B-9120; and ATCC No. HB-1 1430) or react with malignant breast cancer cells but not with normal breast tissue (e.g., ATCC No. HB-8691; ATCC No. HB-10807; and 21HB-108011). Other antibodies or fragments thereof, which react with diseased tissue and not with normal tissue, would be apparent to the skilled artisan.

In another embodiment, the recombinant Rhabdoviridae, contemplated by this invention may express at least one protein, which is immunogenic.

The term “immunogenic”, as used herein, refers to an ability to elicit an immune response. Immune responses that are cell-mediated, or immune responses that are classically referred to as “humoral”, referring to antibody-mediated responses, or both, may be elicited by the recombinant Rhabdoviridae of the present invention.

A recombinant Rhabdovirus of the present invention further encoding for an immunogenic protein or peptide may, in one embodiment, be used for vaccine purposes, as a means of preventing infection.

Recombinant Rhabdoviridae, of which VSV is but one example, are the most promising candidates for vaccine vectors. VSV has a simple genome that contains only five genes. With the advent of reverse genetics it became possible to generate recombinant Rhabdoviridae, which may encode heterologous antigenic proteins, as well as immunomodulatory proteins. The VSV genome can accommodate relatively large insertions without affecting the ability of the virus to replicate or assemble. Due to the rod-shaped morphology of VSV, the ribonucleocapsid core and the virus particle itself is expandable. For example as additional genes are added to the genome, the particles simply get longer (18). Third, the accumulation of mutations in foreign genes inserted into VSV is sufficiently low to allow long-term expression of the foreign gene after numerous viral passages (19). Fourth, since VSV has a non-segmented, negative-strand RNA genome and replication of the virus occurs exclusively in the cytoplasm and involves only RNA intermediates, there is no possibility that the virus genome can integrate into host cell DNA. Therefore, the concern of insertional mutagenesis, which must be considered with other DNA-based vectors, is eliminated. In addition, VSV can productively infect a large variety of different cell types and has the ability to efficiently shut down host cell protein synthesis during its normal replicative cycle, while expressing large quantities of virally-encoded proteins (18-20). In animals, VSV infection has been shown to elicit strong immune responses specific the proteins encoded by recombinant viruses (21). Also, VSV infection of humans is rare in most parts of the world (22), therefore, interference with a VSV-based vaccine by pre-existing immunity would be infrequent.

Non-cytopathic Rhabdoviridae, and Rhabdoviridae diminished in their capacity for cell-to-cell spread are very attractive candidates for use as vaccine delivery vectors. The latter Rhabdoviridae, for example, produce progeny virions released from infected cells that cannot re-infect adjacent cells.

Recombinant Rhabdoviridae of the present invention comprising mutations or deletions in Rhabdoviral M proteins and G proteins, or fragments thereof, and/or in the membrane-proximal ectodomain of G, serve to attenuate the virus. Incorporation of thus mutated or deleted Rhabdoviridae therefore provide a viral vector with enhanced safety factors, for example, and in one embodiment, for use in immunocomprosmised individuals, in applications utilizing the vectors as gene delivery vehicles or vaccines.

In another embodiment, a recombinant Rhabdovirus of the present invention further encoding for an immunogenic protein or peptide may be used as a therapeutic, as a means of halting disease progression, or diminishing the severity of disease.

It may be desirable to incorporate additional attenuating molecules in the constructs of the present invention. In another embodiment, the recombinant Rhabdoviridae contemplated by this invention may express a suicide gene, resulting in cell death, in cells that comprise the products herein.

As used herein, the term “suicide gene” refers to a nucleic acid coding for a product, wherein the product causes cell death by itself or in the presence of other compounds. A representative example of a suicide gene is one, which codes for thymidine kinase of herpes simplex virus. Additional examples are thymidine kinase of varicella zoster virus and the bacterial gene cytosine deaminase, which can convert 5-fluorocytosine to the highly cytotoxic compound 5-fluorouracil.

Suicide genes may produce cytotoxicity by converting a prodrug to a product that is cytotoxic. As used herein “prodrug” means any compound that can be converted to a toxic product for cells. Representative examples of such a prodrug is gancyclovir which is converted in vivo to a toxic compound by HSV-thymidine kinase. The gancyclovir derivative subsequently is toxic to cells. Other representative examples of prodrugs include acyclovir, FIAU [1-(2-deoxy-2-fluoro-β-D-arabinofuranosyl)-5-iodouracil], 6-methoxypurine arabinoside for VZV-TK, and 5-fluorocytosine for cytosine deaminase.

In one embodiment, an added safety factor is provided by the incorporation of a suicide gene within the constructs of the present invention. In another embodiment, the incorporation of suicide genes within cells results in targeted cytotoxicity, which provides a therapeutic protocol when targeted cell lysis is desired. Such incorporation will, in another embodiment, be desirable for anti-cancer applications, whereby cancer cells are specifically targeted via the recombinant Rhabdoviridae of the invention, and cancer cell specific lysis may be affected by incorporation of a suicide gene.

In another embodiment, the recombinant Rhabdoviridae of the present invention are utilized, wherein the recombinants further express an immunogenic protein or polypeptide eliciting a “Th1” response, in a disease where a so-called “Th2” type response has developed, when the development of a so-called Th1” type response is beneficial to the subject. Introduction of the immunogenic protein or polypeptide results in a shift toward a Th1 type response.

As used herein, the term “Th2 type response” refers to a pattern of cytokine expression, elicited by T Helper cells as part of the adaptive immune response, which support the development of a robust antibody response. Typically Th2 type responses are beneficial in helminth infections in a subject, for example. Typically Th2 type responses are recognized by the production of interleukin-4 or interleukin 10, for example.

As used herein, the term “Th1 type response” refers to a pattern of cytokine expression, elicited by T Helper cells as part of the adaptive immune response, which support the development of robust cell-mediated immunity. Typically Th1 type responses are beneficial in intracellular infections in a subject, for example. Typically Th1 type responses are recognized by the production of interleukin-2 or interferon γ, for example.

In another embodiment, the reverse occurs, where a Th1 type response has developed, when Th2 type responses provide a more beneficial outcome to a subject, where introduction of the immunogenic protein or polypeptide via the recombinant viruses/Rhabdoviridae, nucleic acids, vectors or compositions of the present invention provides a shift to the more beneficial cytokine profile. One example would be in leprosy, where the recombinant viruses/Rhabdoviridae, nucleic acids, vectors or compositions of the present invention express an antigen from M. leprae, where the antigen stimulates a Th1 cytokine shift, resulting in tuberculoid leprosy, as opposed to lepromatous leprosy, a much more severe form of the disease, associated with Th2 type responses.

It is to be understood that any use of the recombinant Rhabdoviridae of the present invention expressing an immunogenic protein for purposes of immunizing a subject to prevent disease, and/or ameliorate disease, and/or alter disease progression are to be considered as part of this invention.

Examples of infectious virus to which stimulation of a protective immune response is desirable include: Retroviridae (e.g., human immunodeficiency viruses, such as HIV-1 (also referred to as HTLV-III, LAV or HTLV-III/LAV, or HIV-III; and other isolates, such as HIV-LP; Picornaviridae (e.g., polio viruses, hepatitis A virus; enteroviruses, human coxsackie viruses, rhinoviruses, echoviruses); Calciviridae (e.g., strains that cause gastroenteritis); Togaviridae (e.g., equine encephalitis viruses, rubella viruses); Flaviridae (e.g., dengue viruses, encephalitis viruses, yellow fever viruses); Coronaviridae (e.g., coronaviruses); Rhabdoviridae (e.g., vesicular stomatitis viruses, rabies viruses); Filoviridae (e.g., ebola viruses); Paramyxoviridae (e.g., parainfluenza viruses, mumps virus, measles virus, respiratory syncytial virus); Orthomyxoviridae (e.g. influenza viruses); Bungaviridae (e.g., Hantaan viruses, bunga viruses, phleboviruses and Nairo viruses); Arena viridae (hemorrhagic fever viruses); Reoviridae (erg., reoviruses, orbiviurses and rotaviruses); Birnaviridae; Hepadnaviridae (Hepatitis B virus); Parvoviridae (parvoviruses); Papovaviridae (papilloma viruses, polyoma viruses); Adenoviridae (most adenoviruses); Herpesviridae (herpes simplex virus (HSV) 1 and 2, varicella zoster virus, cytomegalovirus (CMV), herpes viruses'); Poxyiridae (variola viruses, vaccinia viruses, pox viruses); and Iridoviridae (e.g. African swine fever virus); and unclassified viruses (e.g., the etiological agents of Spongiform encephalopathies, the agent of delta hepatities (thought to be a defective satellite of hepatitis B virus), the agents of non-A, non-B hepatitis (class 1=internally transmitted; class 2=parenterally transmitted (i.e., Hepatitis C); Norwalk and related viruses, and astroviruses).

Examples of infectious bacteria to which stimulation of a protective immune response is desirable include: Helicobacter pylori, Borellia burgdorferi, Legionella pneumophilia, Mycobacteria sps (e.g. M. tuberculosis, M. avium, M. intracellulare, M. kansaii, M. gordonae), Staphylococcus aureus, Neisseria gonorrhoeae, Neisseria meningitidis, Listeria monocytogenes, Streptococcus pyogenes (Group A Streptococcus), Streptococcus agalactiae (Group B Streptococcus), Streptococcus (viridans group), Streptococcus faecalis, Streptococcus bovis, Streptococcus (anaerobic sps.), Streptococcus pneumoniae, pathogenic Campylobacter sp., Enterococcus sp., Chlamidia sp., Haemophilus influenzae, Bacillus antracis, corynebacterium diphtheriae, corynebacterium sp., Erysipelothrix rhusiopathiae, Clostridium perfringers, Clostridium tetani, Enterobacter aerogenes, Klebsiella pneumoniae, Pasturella multocida, Bacteroides sp., Fusobacterium nucleatum, Streptobacillus moniliformis, Treponema pallidium, Treponema pertenue, Leptospira, Actinomyces israelli and Francisella tularensis.

Examples of infectious fingi to which stimulation of a protective immune response is desirable include: Cryptococcus neoformans, Histoplasma capsulatum, Coccidioides immitis, Blastomyces dermatitidis, Chlamydia trachomatis, Candida albicans. Other infectious organisms (i.e., protists) include: Plasmodium sp., Leishmania sp., Schistosoma sp. and Toxoplasma sp.

In another embodiment, the recombinant Rhabdoviridae of the present invention expressing an immunogenic protein further express additional immunomodulating proteins.

Examples of useful immunomodulating proteins include cytokines, chemokines, complement components, immune system accessory and adhesion molecules and their receptors of human or non-human animal specificity. Useful examples include GM-CSF, IL-2, IL-12, OX40, OX40L (gp34), lymphotactin, CD40, and CD40L. Further useful examples include interleukins for example interleukins 1 to 15, interferons alpha, beta or gamma, tumour necrosis factor, granulocyte-macrophage colony stimulating factor (GM-CSF), macrophage colony stimulating factor (M-CSF), granulocyte colony stimulating factor (G-CSF), chemokines such as neutrophil activating protein (NAP), macrophage chemoattractant and activating factor (MCAF), RANTES, macrophage inflammatory peptides MIP-1a and MIP-1b, complement components and their receptors, or an accessory molecule such as B7.1, B7.2, TRAP, ICAM-1, 2 or 3 and cytokine receptors. OX40 and OX40-ligand (gp34) are further useful examples of immunomodulatory proteins.

In another embodiment, the immunomodulatory proteins may be of human or non-human animal specificity, and may comprise extracellular domains and/or other fragments with comparable binding activity to the naturally occurring proteins. Immunomodulatory proteins may, in another embodiment, comprise mutated versions of the embodiments listed, or comprise fusion proteins with polypeptide sequences, such as immunoglobulin heavy chain constant domains. Multiple immunomodulatory proteins may be incorporated within a single construct, and as such, represents an additional embodiment of the invention.

It is to be understood that the recombinant Rhabdoviridae of the present invention may express multiple immunogenic proteins. In one embodiment, the immunogenic proteins or peptides are derived from the same or related species. Vaccine incorporation of multiple antigens has been shown to provide enhanced immunogenicity.

The recombinant Rhabdoviridae of the present invention expressing an immunogenic protein or peptide fragment may generate immune responses of a variety of types that can be stimulated by the constructs, including responses against the heterologously expressed protein or peptide, other antigens that are now immunogenic via a “by-stander” effect, against host antigens, and others, and represent additional embodiments of the invention. It is envisioned that methods of the present invention can be used to prevent or treat bacterial, viral, parasitic or other disease states, including tumors, in a subject.

Combination vaccines have been shown to provide enhanced immunogenicity and protection, and, as such, in another embodiment, the immunogenic proteins or peptides are derived from different species.

In another embodiment, the invention provides a recombinant virus comprising a nucleic acid of a Rhabdovirus genome, or a fragment thereof, wherein said Rhabdovirus genome or fragment thereof comprises a deletion or a mutation within a region encoding a matrix protein (M). The Rhabdovirus genome or fragment thereof, and deleted or mutated Rhabdoviral Matrix protein in the recombinant virus may comprise all embodiments listed herein.

In another embodiment, the invention provides a recombinant virus comprising a nucleic acid of a Rhabdovirus genome, or a fragment thereof, wherein said Rhabdovirus genome or fragment thereof comprises a deletion or a mutation within a region encoding a glycoprotein (G), in addition to a mutation in the Rhabdoviral M protein. In another embodiment, the invention provides a recombinant virus comprising a nucleic acid of a Rhabdovirus genome, wherein said Rhabdovirus genome comprises a deletion or a mutation within a region encoding a membrane-proximal ectodomain of the glycoprotein (G). In another embodiment, the recombinant Rhabdovirus comprises a deletion or a mutation within a region encoding a membrane-proximal ectodomain of the glycoprotein (G) in addition to a mutation in the Rhabdoviral M protein. The recombinant viruses herein described may comprise all embodiments listed in regard to recombinant Rhabdoviridae of this invention, and represent additional embodiments of this invention.

In another embodiment, the invention provides an isolated nucleic acid molecule comprising a polynucleotide sequence encoding a genome of a non-cytopathic Rhabdovirus, the polynucleotide sequence comprising a deletion or a mutation in a gene encoding a matrix protein (M). It is to be understood that the isolated nucleic acid molecule may comprise all embodiments listed herein, including sequences encoding for heterolgous protein expression, G stem polypeptide and fusion facilitating polypeptide expression, and deletions in G glycoprotein expression and deletions in the membrane-proximal ectodomain of the glycoprotein, each of which represents an additional embodiment of the present invention. In another embodiment, the invention provides a vector comprising the isolated nucleic acid molecules described herein.

In another embodiment, this invention provides an isolated nucleic acid molecule comprising a polynucleotide sequence encoding a genome of a Rhabdovirus, wherein the polynucleotide sequence has a deletion or a mutation in a gene encoding a membrane-proximal ectodomain of the glycoprotein (G). The isolated nucleic acid molecule according to this aspect of the invention may comprise embodiments listed herein, including sequences encoding for heterolgous protein expression, fusion facilitating polypeptide expression, and mutations or deletions in matrix protein expression, each of which represents an additional embodiment of the present invention. In another embodiment, the invention provides a vector comprising such an isolated nucleic acid molecule.

As used herein, the term “nucleic acid” molecule can include, but is not limited to, prokaryotic sequences, eukaryotic mRNA, cDNA from eukaryotic mRNA, genomic DNA sequences from eukaryotic (e.g., mammalian) DNA, and even synthetic DNA sequences. The term also refers to sequences that include any of the known base analogs of DNA and RNA.

The nucleic acid sequences described herein may be subcloned within a particular vector, depending upon the desired method of introduction of the sequence within cells. Once the nucleic acid segment is subcloned into a particular vector it thereby becomes a recombinant vector. To generate the nucleic acid constructs in context of the present invention, the polynucleotide segments encoding sequences of interest can be ligated into commercially available expression vector systems suitable for transducing/transforming mammalian cells and for directing the expression of recombinant products within the transduced cells. It will be appreciated that such commercially available vector systems can easily be modified via commonly used recombinant techniques in order to replace, duplicate or mutate existing promoter or enhancer sequences and/or introduce any additional polynucleotide sequences such as for example, sequences encoding additional selection markers or sequences encoding reporter polypeptides.

There are a number of techniques known in the art for introducing the above described recombinant vectors into cells of the present invention, such as, but not limited to: direct DNA uptake techniques, and virus, plasmid, linear DNA or liposome mediated transduction, receptor-mediated uptake and magnetoporation methods employing calcium-phosphate mediated and DEAE-dextran mediated methods of introduction, electroporation, liposome-mediated transfection, direct injection, and receptor-mediated uptake (for further detail see, for example, “Methods in Enzymology” Vol. 1-317, Academic Press, Current Protocols in Molecular Biology, Ausubel F. M. et al. (eds.) Greene Publishing Associates, (1989) and in Molecular Cloning: A Laboratory Manual, 2nd Edition, Sambrook et al. Cold Spring Harbor Laboratory Press, (1989), or other standard laboratory manuals). Bombardment with nucleic acid coated particles is also envisaged.

The efficacy of a particular expression vector system and method of introducing nucleic acid into a cell can be assessed by standard approaches routinely used in the art. For example, DNA introduced into a cell can be detected by a filter hybridization technique (e.g., Southern blotting) and RNA produced by transcription of introduced DNA can be detected, for example, by Northern blotting, RNase protection or reverse transcriptase-polymerase chain reaction (RT-PCR). The gene product can be detected by an appropriate assay, for example by immunological detection of a produced protein, such as with a specific antibody, or by a functional assay to detect a functional activity of the gene product, such as an enzymatic assay. If the gene product of interest to be expressed by a cell is not readily assayable, an expression system can first be optimized using a reporter gene linked to the regulatory elements and vector to be used. The reporter gene encodes a gene product, which is easily detectable and, thus, can be used to evaluate efficacy of the system. Standard reporter genes used in the art include genes encoding β-galactosidase, chloramphenicol acetyl transferase, luciferase and human growth hormone, or any of the marker proteins listed herein.

In another embodiment, a packaging system is constructed, comprising cDNA comprising mutations or deletions in Rhabdoviral M proteins, which may serve as a further means of attenuation.

A packaging system is a vector, or a plurality of vectors, which collectively provide in expressible form all of the genetic information required to produce a virion which can encapsidate the nucleic acid, transport it from the virion-producing cell, transmit it to a target cell, and, in the target cell, facilitate transgene expression. However, the packaging system must be substantially incapable of packaging itself, hence providing a means of attenuation, since virion production, following introduction into target cells is prevented.

In another embodiment, this invention provides cells comprising the recombinant Rhabdoviridae, viruses, vectors or nucleic acids described herein. In one embodiment, the cell is prokaryotic, or in another embodiment, eukaryotic. It is to be understood that each embodiment listed herein for the recombinant Rhabdoviridae, viruses vectors and/or nucleic acids may be incorporated within cells, and represent envisaged parts of this invention. Recombinant Rhabdoviral or viral particles are similarly additional embodiments of this invention, and may comprise any permutation as listed herein.

Thus recombinant Rhabdoviridae and/or particles can be prepared, assembled and isolated. In one embodiment, the recombinant Rhabdoviridae and/or particles thus prepared are not cytopathic.

In another embodiment, mutations or deletions in the Rhabdoviral M protein produce a virus with cytopathic effects only in highly malignant cells. Use of such Rhabdoviral strains may provide a preferential means of specific malignant cell lysis, without effect on neighboring cells. In another embodiment, incorporation of mutations or deletions in the Rhabdoviral M protein is a means of further attenuating any construct incorporating a Rhabdoviral genome, as its cytotoxic effect is restricted to highly malignant cells alone.

Methods for generating recombinant Rhabdoviruses may entail utilizing cDNAs and a Minivirus or a Helper Cell Line. In this case, both “miniviruses” and “helper cells” (also known as “helper cell lines”) provide a source of Rhabdoviral proteins for Rhabdovirus virion assembly, which are not produced from the transfected DNA encoding genes for Rhabdoviral proteins.

The generation of recombinant Rhabdovirus can be accomplished using: (1) cDNA's alone; (2) cDNAs transfected into a helper cell in combinations; or (3) cDNA transfection into a cell, which is further infected with a minivirus providing in trans the remaining components or activities needed to produce either an infectious or non-infectious recombinant Rhabdovirus. Using any of these methods (e.g., minivirus, helper cell line, or cDNA transfection only), the minimum components required are an RNA molecule containing the cis-acting signals for (1) encapsidation of the genomic (or antigenomic) RNA by the Rhabdovirus N protein, and (2) replication of a genomic or antigenomic (replicative intermediate) RNA equivalent. The DNA needed to make a recombinant Rhabdovirus: The phrase “cDNA's necessary” to produce an infectious Rhabdovirus means the nucleic acid molecules required to produce infectious recombinant Rhabdovirus particles that express a mutated matrix protein (M).

The term “minivirus” is meant to include incomplete viral particles containing a polycistronic nucleic acid molecule encoding N-P-M-L, N-P-L, N-P-G-L, M-G, G only, M only or any combination of four or fewer Rhabdoviral genes. This incomplete virus particle is incapable of viral replication, a process of the Rhabdoviral lifecycle involving a complete copying of its genome.

Copying of the Rhabdoviral genome, referred to as “Rhabdoviral replication” requires, the presence of a replicating element or replicon, which, herein signifies a strand of RNA minimally containing at the 5′ and 3′ ends the leader sequence and the trailer sequence of a Rhabdovirus. In the genomic sense, the leader is at the 3′ end and the trailer is at the 5′ end. Any RNA placed between these two replication signals will in turn be replicated. The leader and trailer regions further must contain the minimal cis-acting elements for purposes of encapsidation by the N protein and for polymerase binding, which are necessary for initiating Rhabdoviral transcription and replication.

Several different VSV-derived replicons have been generated and have been shown to replicate and express heterologous (non-VSV) proteins for prolonged periods in cultured cells. Such replicons are ideal candidates for gene therapy vectors because they replicate exclusively in the cytoplasm, which eliminates the concern of insertional mutagenesis into the target cell chromosome posed by other gene therapy vectors. In addition, there has been no evidence that VSV-based replicons can undergo homologous (or heterologous) recombination, despite extensive attempts to document any type of recombination in infected cells. The inability to recombine eliminates the concern that replication and infectious competence may be restored by infection of cells containing the replicon with other negative-strand RNA viruses.

In order to produce the recombinant Rhabdovirus of the present invention the cDNA's encoding the modified Rhabdoviral genome listed above must be contacted with a cell under conditions facilitating expression of the vectors employed, permitting production of the recombinant Rhabdovirus. It is to be understood that any cell permitting assembly of the recombinant Rhabdovirus for any one of the three methods disclosed above are included as part of the present invention.

Culturing of Cells to Produce Virus: Transfected cells are usually incubated for at least 24 hours at the desired temperature, usually about 37° C. For generation of infectious virus particles, the supernatant, which contains recombinant virus is harvested and transferred to fresh cells. The fresh cells expressing the G protein (either via transient or stable transfection) are incubated for approximately 48 hours, and the supernatant is collected.

Purification of the Recombinant Rhabdovirus: The terms “isolation” or “isolating” a Rhabdovirus signifies the process of culturing and purifying virus particles such that very little cellular debris remains. One example would be to collect the virion-containing supernatant and filter (0.2μ pore size) (e.g., Millex-GS, Millipore) the supernatant thus removing Vaccinia virus and cellular debris. Alternatively, virions can be purified using a gradient, such as a sucrose gradient. Recombinant Rhabdovirus particles can then be pelleted and resuspended in whatever excipient or carrier is desired. Viral titers can be determined by serial dilution of supernatant used to infect cells, whereupon following expression of viral proteins, infected cells are quantified via indirect immunofluorescence using for example, anti-M (23H12) or anti-N (10G4) protein specific antibodies (L. Lefrancois et al., (1982) Virology 121: 157-67). It is therefore to be understood that in recombinant Rhabdoviral particles are considered as part of the invention, as well.

In another embodiment, this invention provides a method of producing a non-cytopathic recombinant Rhabdovirus comprising a genetically modified nucleic acid encoding Rhabdovirus proteins including a mutation or a deletion within a matrix protein (M) comprising the steps of: (A) inserting into a suitable cell a polynucleotide sequence encoding Rhabdoviral proteins including a mutation or a deletion within the matrix protein (M), a polynucleotide sequence encoding a marker polypeptide and a polycistronic cDNA comprising at least the 3′ and 5′ Rhabdovirus leader and trailer regions containing the cis acting signals for Rhabdovirus replication; (B) culturing the cell under conditions that select for a noncytopathic phenotype of said cell; (C) culturing said cell under conditions that permit production of the recombinant Rhabdovirus, and (D) isolating said non cytopathic recombinant Rhabdovirus.

In one embodiment, the method includes a step of isolating genomic RNA from the isolated recombinant Rhabdoviridae of this invention. In another embodiment, the step of isolating genomic RNA is performed via RT-PCR. In another embodiment, the cells utilized for the production methods are selected from the group consisting of rodent, primate and human cells.

In another embodiment, non-cytopathic recombinant Rhabdoviridae with mutations or deletions in the G glycoprotein are produced, via the methods described herein. According to this aspect of the invention, a polynucleotide sequence encoding Rhabdoviral proteins including a mutation or a deletion within the glycoprotein (G) are inserted into the cell, as described. In one embodiment, the mutation or deletion in the glycoprotein is in the membrane-proximal ectodomain of the glycoprotein.

In another embodiment, non-cytopathic recombinant Rhabdoviridae further expressing a heterologous nucleic acid sequence encoding a second polypeptide are produced, via the methods described herein. According to this aspect of the invention, a polynucleotide sequence encoding at least one heterologous polypeptide is inserted into the cell, as described. In one embodiment, the second polypeptide is a therapeutic polypeptide. In another embodiment, the second peptide is immunogenic.

In another embodiment, this invention provides a method of producing a recombinant Rhabdovirus comprising a genetically modified nucleic acid encoding Rhabdoviral proteins comprising a deletion or a mutation within a membrane-proximal ectodomain of a glycoprotein (G) comprising the steps of: (A) inserting into a suitable cell a polynucleotide sequence encoding Rhabdovirus proteins including a deletion or a mutation within the membrane-proximal ectodomain of the glycoprotein (G), a polynucleotide sequence encoding a marker polypeptide and a polycistronic cDNA comprising at least the 3′ and 5′ Rhabdovirus leader and trailer regions containing the cis acting signals for Rhabdovirus replication; (B) culturing the cell under conditions that permit production of the recombinant Rhabdovirus, and (C) isolating the recombinant Rhabdovirus.

For purposes of infecting cells (such as, for example, tissue culture cells or cells from a tissue sample, such as a biopsy), the isolated recombinant Rhabdovirus is incubated with the cells using techniques known in the art. Detection of infection by the recombinant Rhabdovirus could proceed by determining the presence of a reporter gene, such as a green fluorescent protein (GFP), or via assessment of viral protein expression, as determined by indirect immunofluorescence, as discussed above.

To prepare infectious virus particles, an appropriate cell line (e.g., BHK cells) is first infected with vaccinia virus vTF7-3 (T. R. Fuerst et al., (1986) Proc. Natl. Acad. Sci. USA 3. 8122-26) or equivalent which encodes a T7 RNA polymerase or other suitable bacteriophage polymerase such as the T3 or SP6 polymerases (see Usdin et al., (1993) BioTechniques 14:222-224 or Rodriguez et al. (1990) J. Virol. 64:4851-4857). Alternatively, a vaccinia-free system may be utilized which provides an RNA polymerase. The cells are then transfected with individual cDNA containing the genes encoding the N, P, G and L Rhabdoviral proteins. These cDNAs will provide the proteins for building the recombinant Rhabdovirus particle. Cells can be transfected by any method known in the art (e.g., liposomes, electroporation, etc.).

The invention further relates to diagnostic and pharmaceutical packs and kits comprising one or more containers filled with one or more of the ingredients of the aforementioned vectors, viruses or compositions of the invention.

In another embodiment, the invention provides compositions comprising the recombinant viruses, Rhabdoviridae, nucleic acids or vectors described herein, for administration to a cell or to a multi cellular organism. The vectors of the invention may be employed, in another embodiment, in combination with a non-sterile or sterile carrier or carriers for administration to cells, tissues or organisms, such as a pharmaceutical carrier suitable for administration to an individual. Such compositions comprise, for instance, a media additive or a therapeutically effective amount of a recombinant virus of the invention and a pharmaceutically acceptable carrier or excipient. Such carriers may include, but are not limited to, saline, buffered saline, dextrose, water, glycerol, and combinations thereof. The formulation should suit the mode of administration.

The recombinant viruses, vectors or compositions of the invention may be employed alone or in conjunction with other compounds, such as additional therapeutic compounds.

The pharmaceutical compositions may be administered in any effective, convenient manner including, for instance, administration by intravascular (i.v.), intramuscular (i.m.), intranasal (i.n.), subcutaneous (s.c.), oral, rectal, intravaginal delivery, or by any means in which the recombinant virus/composition can be delivered to tissue (e.g., needle or catheter). Alternatively, topical administration may be desired for insertion into epithelial cells. Another method of administration is via aspiration or aerosol formulation.

For administration to mammals, and particularly humans, it is expected that the physician will determine the actual dosage and duration of treatment, which will be most suitable for an individual and can vary with the age, weight and response of the particular individual.

The routes of administration utilized for recombinant Rhabdoviruses facilitate viral circulation, attachment and infection, thereby enabling viral expression of encoded proteins, which may be assayed via the incorporation of reporter proteins within the recombinant Rhabdovirus. It is expected that following Rhabdoviral administration viral protein expression, (as determined, for example, by reporter protein detection) should occur within 24 hours and certainly within 36 hours.

In another embodiment, the invention provides a method for immunizing a subject against a disease comprising the step of contacting a target cell of said subject with a therapeutically effective amount of a recombinant virus, wherein the virus comprises a Rhabdoviral genome, or fragment thereof, the Rhabdoviral genome or fragment thereof including a deletion or a mutation within a region encoding a matrix protein (M) and a heterologous gene encoding an immunogenic protein, or peptide fragment, capable of being expressed inside the target cell, thereby immunizing against a disease.

As used herein, the term “contacting a target cell” refers to both direct and indirect exposure of the target cell to a virus, nucleic acid, vector or composition of the invention. In one embodiment, contacting a cell may comprise direct injection of the cell through any means well known in the art, such as microinjection. It is also envisaged, in another embodiment, that supply to the cell is indirect, such as via provision in a culture medium that surrounds the cell.

Protocols for introducing the viruses, nucleic acids or vectors of the invention into target cells may comprise, for example: direct DNA uptake techniques, virus, plasmid, linear DNA or liposome mediated transduction, or transfection, magnetoporation methods employing calcium-phosphate mediated and DEAE-dextran mediated methods of introduction, electroporation, direct injection, and receptor-mediated uptake (for further detail see, for example, “Methods in Enzymology” Vol. 1-317, Academic Press, Current Protocols in Molecular Biology, Ausubel F. M. et al. (eds.) Greene Publishing Associates, (1989) and in Molecular Cloning: A Laboratory Manual, 2nd Edition, Sambrook et al. Cold Spring Harbor Laboratory Press, (1989), or other standard laboratory manuals). It is to be understood that any direct means or indirect means of intracellular access of a virus, nucleic acid or vector of the invention is contemplated herein, and represents an embodiment thereof.

In another embodiment, the invention provides a method for treating a subject suffering from a disease comprising the step of contacting a target cell of the subject with a therapeutically effective amount of a recombinant virus, wherein the virus comprises a Rhabdoviral genome, or fragment thereof, said Rhabdoviral genome or fragment thereof including a deletion or a mutation within a region encoding a matrix protein (M) and a heterologous gene encoding an immunogenic protein or peptide fragment, capable of being expressed inside the target cell, thereby treating a disease.

According to this aspect of the invention, in additional embodiments, the target cell is an epithelial cell, a lung cell, a kidney cell, a liver cell, an astrocyte, a glial cell, a prostate cell, a professional antigen presenting cell, a lymphocyte or an M cell.

In another embodiment, the invention provides a method for treating a subject suffering from a disease associated with a defective gene comprising the step of contacting a target cell of the subject with a therapeutically effective amount of a recombinant non-cytopathic Rhabdovirus, or a recombinant virus, vector or cell of the present invention, comprising a Rhabdoviral genome, or a nucleic acid sequence encoding for a Rhabdoviral genome, wherein the genome of the Rhabdovirus includes a deletion or a mutation within a region encoding a matrix protein (M) and a heterologous gene capable of being expressed inside the target cell, thereby treating the disease.

In another embodiment, according to this aspect of the invention, the recombinant non-cytopathic Rhabdovirus may further comprise a mutation or deletion in a membrane-proximal ectodomain the Rhabdoviral glycoprotein.

It is to be understood that the recombinant Rhabdoviridae, viruses, vectors or cells thus utilized may comprise any of the embodiments listed herein, or combinations thereof.

According to this aspect of the invention, the disease for which the subject is thus treated may comprise, but is not limited to: muscular dystrophy, cancer, cardiovascular disease, hypertension, infection, renal disease, neurodegenerative disease, such as alzheimer's disease, parkinson's disease, huntington's chorea, Creuztfeld-Jacob disease, autoimmune disease, such as lupus, rheumatoid arthritis, endocarditis, Graves' disease or ALD, respiratory disease such as asthma or cystic fibrosis, bone disease, such as osteoporosis, joint disease, liver disease, disease of the skin, such as psoriasis or eczema, ophthalmic disease, otolaryngeal disease, other neurological disease such as Turret syndrome, schizophrenia, depression, autism, or stoke, or metabolic disease such as a glycogen storage disease or diabetes. It is to be understood that any disease whereby expression of a particular protein which can be accomplished via the use of the recombinant Rhabdoviridae, viruses, vectors or cells or compositions of this invention is sought, is to be considered as part of this invention.

In one embodiment, the target cell according to this aspect of the invention is an epithelial cell, a lung cell, a kidney cell, a liver cell, an astrocyte, a glial cell or a prostate cell. The method according to this aspect of the invention may provide any of the therapeutic applications further described hereinabove, each of which represents an additional embodiment of the invention. It is to be understood, that any use of the recombinant viruses, cells, vectors, nucleic acids or compositions disclosed herein for any therapeutic application is to be considered envisioned as part of this invention and embodiments thereof.

In another embodiment, this invention provides a method for immunizing a subject against a disease comprising the step of contacting a target cell of the subject with a therapeutically effective amount of a recombinant virus, wherein the virus comprises a Rhabdoviral genome, or fragment thereof, said Rhabdoviral genome or fragment thereof including a deletion or a mutation within a region encoding a matrix protein (M) and/or a mutation or a deletion in a membrane-proximal ectodomain region of a glycoprotein (G) and a heterologous gene encoding an immunogenic protein, or peptide fragment, capable of being expressed inside the target cell, thereby immunizing against a disease.

The recombinant Rhabdoviridae of the invention, and viruses, vectors, cells and compositions herein described, can serve, in another embodiment, as effective anti-cancer therapies. Cancerous cells, such as C6 glioma cells infected with varying amounts of rVSV resulted in roughly 90% cell death within 72 hours (Example 12). However, damage to healthy cells in the culture was evident, as a result of VSV infection. Recombinant VSV deleted for G, in this context, had no effect on neighboring healthy cells, with the viral lytic effect specific for glioma cells.

According to this aspect of the invention, and in another embodiment, there is provided a method for cancer cell lysis, comprising the steps of contacting a cancerous cell with a recombinant Rhabdovirus of this invention, wherein the Rhabdovirus comprises (a) a nucleic acid comprising a Rhabdoviral genome, wherein the Rhabdoviral genome comprises a deletion or a mutation within a region encoding a matrix protein (M) and/or a deletion or a mutation within a region encoding the membrane-proximal ectodomain of a Rhabdoviral glycoprotein (G); and (b) a non-Rhabdoviral nucleic acid.

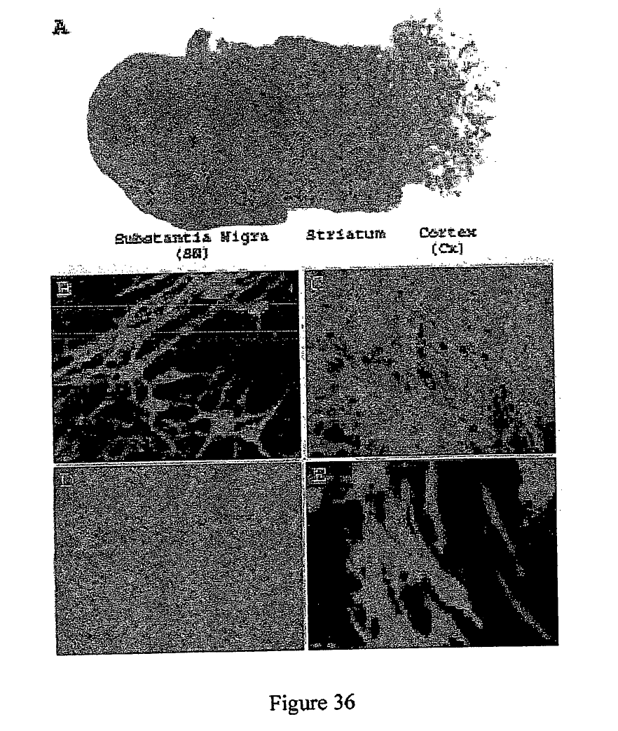

Interferon-β pretreatment resulted in specific glioma cell lysis, (FIG. 36), with very little infection of normal neuronal cells in the slice itself, following infection with VSV.

Thus, in other embodiments, the non-Rhabdoviral nucleic acid encodes for a cytokine or suicide gene. In another embodiment, an additional therapeutic compound is contacted with the cell prior to, during of following infection with the recombinanat Rhabdoviridae of this invention. In one embodiment, the therapeutic compound is a nucleoside analog. In another embodiment, the therapeutic compound is a cytoskeletal inhibitor, such as for example, a microtubule inhibitor.

In one embodiment, the cancerous cell comprises diffuse, or in another embodiment, solid cancerous tissue cell types. In another embodiment, the cancerous cell may be at any stage of oncogenesis, and of any origin.

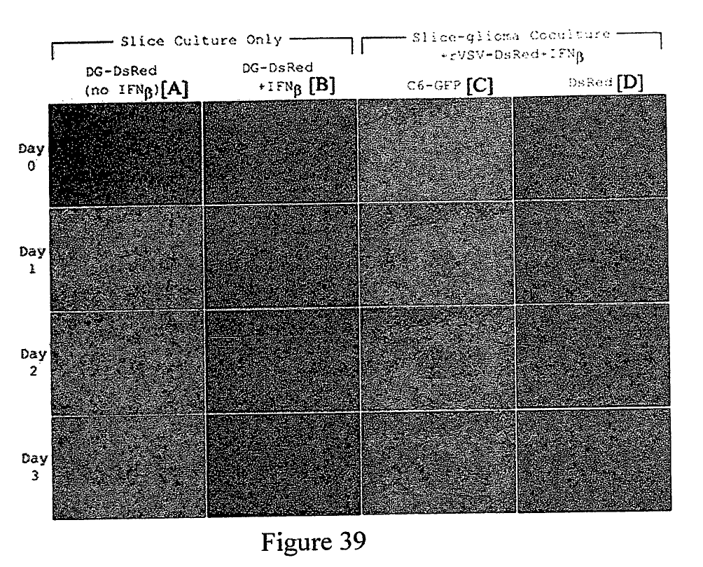

In addition, VSV strains that are deleted for G expression (ΔG) demonstrated significant reduction in tumor load in vivo, following infection with the virus, yet little if any infection of normal cells in the slice culture itself occurred (Example 13, FIG. 39D).

Thus, the invention provides, in another embodiment, a method for treating cancer, comprising the steps of contacting a cancerous cell with a recombinant virus, wherein said virus comprises (a) a nucleic acid comprising a Rhabdoviral genome, or fragment thereof, said Rhabdoviral genome or fragment thereof comprises a deletion or a mutation within a region encoding a matrix protein (M) and/or a deletion or a mutation within a region encoding the membrane-proximal ectodomain of a glycoprotein (G); and (b) a non-Rhabdoviral nucleic acid. In other embodiments, the non-Rhabdoviral nucleic acid encodes for a cytokine or suicide gene.

In another embodiment, this invention provides a model for studying oncogenesis in nervous tissue, comprising the steps of: obtaining vibrotome slices of corona, substantia negra and cortex tissue, culturing said slices on coverslips under conditions maintaining viability and inhibiting mitosis, inoculating said slice culture with labeled cancer cells and determining the fate of the labeled cancer cells.

In one embodiment, the model further comprises the step of inoculating the slice culture with a recombinant Rhabdovirus. In another embodiment, the recombinant Rhabdovirus is mutated or deleted for a Rhabdoviral M protein. In another embodiment, the recombinant Rhabdovirus mutated or deleted for a nucleotide sequence encoding for a Rhabdoviral M protein is further mutated or deleted for a nucleotide sequence encoding for a Rhabdoviral G protein. In another embodiment, the recombinant Rhabdovirus is mutated for a membrane-proximal ectodomain of a Rhabdoviral G protein.

In another embodiment, the model utilizes cancerous cells, which are labeled with a fluorescent, luminescent, chromogenic or electron dense material. In another embodiment, the model utilizes labeled recombinant Rhabdovirus. In another embodiment, an agent thought to augment or inhibit oncogenesis is supplied to the culture, and effects on labeled cancerous cells are determined.

In one embodiment, the agent is a cytokine, chemokine, proinflammatory molecule, an angiogenic factor, an angiogenesis inhibitor, an ionophore, an inhibitor of microtubules or a cell cycle inhibitor.

In another embodiment, agents that alter oncogenesis or are suspected to alter oncogenesis are evaluated in the context of the model provided herein. According to this aspect of the invention, the agent is supplied to the slice culture, following or concurrent with the addition of cancerous cells. In one embodiment, effects on cancer cell viability are determined. In another embodiment, effects on cancer cell proliferation are determined. In another embodiment, effects on cancer cell surface marker expression or cell cyle stage are determined. Such effects are readily measured by methods well known to one skilled in the art, and comprise, but are not limited to: measurements of dye uptake as a measurement of viability, such as, for example, trypan blue exclusion, measurements of cell proliferation can be determined by, for example measurements of ³H-Thymidine uptake, and cell surface marker expression and cell cycle stage can be determined by FACS, and other methods, according to protocols well known to one skilled in the art.

In another embodiment, according to this aspect of the invention, the slices are cultured on coverslips under conditions maintaining viability in a medium comprising Gey's/dextrose solution, plasma, thrombin, Eagle's basal medium, Hanks' balanced salt solution, L-glutamine, or any combination thereof. In another embodiment, according to this aspect of the invention, the slices are cultured on coverslips under conditions inhibiting mitosis, in a medium comprising cytosine-a-D-.arabinofuranoside, uridine, 5-fluro-2′-deoxyuridine, Gey's/dextrose solution, plasma, thrombin, Eagle's basal medium, Hanks' balanced salt solution, L-glutamine or any combination thereof.

The model, in one embodiment, allows for the analysis of toxicity to normal tissues and efficacy of potential tumor therapies, which can be studied simultaneously and, in another embodiment, in real-time. The organotypic slice culture allows, in one embodiment, for maintenance of appropriate neuronal architecture, in terms of antaomical connections normally be present in vivo, during the course of any given study utilizing the model, and thus realistically approximates cellular, architectural, and physiological aspects of the in vivo brain (23-32). The model may be used, in other embodiments, for studies in which pharmacological, physiological and structural studies of brain tissue are desired, and may be used as a source of comparison, in another embodiment, with similar studies conducted in vivo.

In another embodiment, the culture system of the model may be utilized for short-term studies, or in another embodiment, for long-term studies, without loss of cell integrity or electrophysiological responsiveness.

In another embodiment, this invention provides a method for identifying an agent that has oncolytic activity, comprising the steps of: obtaining vibrotome slices of corona, substantia negra and cortex tissue, culturing said slices on coverslips under conditions maintaining viability and inhibiting mitosis, inoculating said slice culture with labeled cancer cells, culturing said inoculated culture with a candidate agent, and determining cancer cell viability, wherein a decrease in cancer cell viability indicates that the candidate agent is oncolytic, thereby identifying an agent that has oncolytic activity.

In one embodiment, the cancerous cells are of neuronal origin, for example, glioma cells. In another embodiment, according to this aspect of the invention, the cancerous cells may be labeled with a fluorescent, luminescent, chromogenic or electron dense label. In another embodiment, the method further comprises the step of inoculating the slice culture with labeled recombinant Rhabdovirus.

In another embodiment, this method according to this aspect of the invention further comprises the step of culturing the inoculated slice culture with a cytokine. In one embodiment, the cytokine is an interferon, an interleukin, a chemoattractant, such as tumor necrosis factor, or migration inhibition factor or macrophage inflammatory protein.

It is to be understood that any embodiment listed herein for recombinant Rhabdoviridae, and for the slice model represent additional embodiments of the method for identifying an agent with oncolytic activity.

In another embodiment, the recombinant viruses, nucleic acids or vectors of the invention that express a heterologous protein may be utilized as a protein expression system. Stable introduction of the constructs within cells may provide a means for high yield production of the expressed protein.

The following examples are presented in order to more fully illustrate some embodiments of the invention. They should, in no way be construed, however, as limiting the scope of the invention.

### EXAMPLES

### Example 1

### Mutations in the M Protein of VSV Produce Infectious Yet Non-Cytopathic Virus

### Materials and Methods

Site-directed mutagenesis of VSV expressing the GFP gene was conducted. BHK-21 cells were then infected with the mutated VSV. Viral particles were concentrated from cell culture supernatants by ultracentrifugation and viral RNA was isolated. Reverse transcription-PCR was used to obtain a full-length cDNA of the NCP-12 variant. M gene and the cDNA were subjected to automated sequence analysis.

Infected BHK-21 (MOI of 10) cells cultured and cell infectivity and morphology was determined via fluorescence microscopy, at indicated times. Rounded cells were aspirated from the culture and cultures were washed several times with gentle pipetting, then incubated, and examined periodically. After 7 days cultures were examined for the presence of GFP-positive cells, indicating infection, and culture supernatants were harvested with aliquots used to infect fresh cells. Cells were examined for GFP expression, 24 hours post infection with culture supernatants. Cells were also fixed with 3% paraformaldehyde, permeabilized with 1% Triton X-100 followed by probing for N protein with an N protein-specific monoclonal antibody (10G4, Lefrancois and Lyles, (1982) were Virology 121:157-167.) conjugated to Alexa568 dye (Molecular Probes), for evidence of infection.

BHK-21 cells were also grown on coverslips and transiently transfected with 2 μgs of pCAGGS-M wt, pCAGGS-NCP-12.1, or pCAGGS-MCS plasmids using 10 μl lipofectamine (Gibco BRL) in Optimem (Gibco BRL)). At 24 hours post transfection, cells were fixed with 3% paraformaldehyde, permeabilized with 1% Triton X-100 and probed for M protein expression with an M-specific monoclonal antibody (23H12) labeled with a rhodamine conjugated goat anti-mouse secondary antibody.

M_{NCP12.1.} cDNA was cloned for the M gene containing the four identified mutations, and the mutant gene was replaced with the wild-type M gene to determine recombinant virus recovery via standard procedures, with the phenotype of the recovered viruses examined microscopically.

### Results

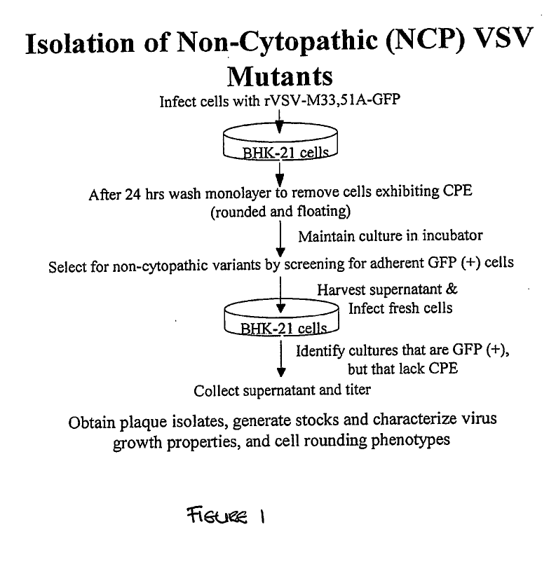

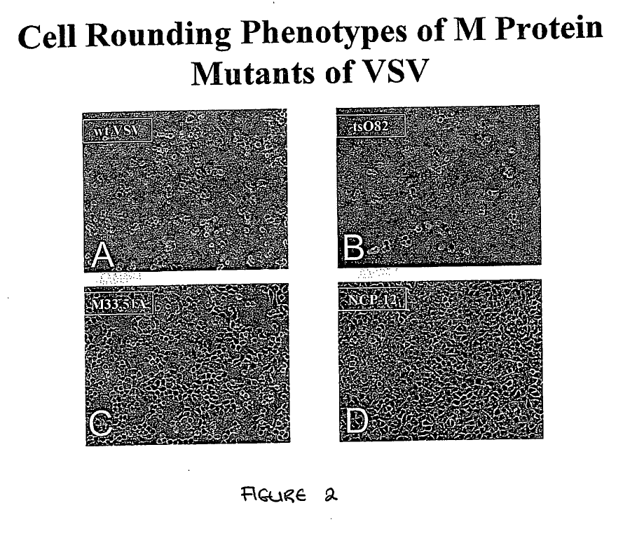

Previous evidence suggested that two N-terminally truncated M proteins (called M2 and M3) contribute to the cytopathic effect (CPE) seen in VSV infected cells. Isolation of VSV M protein mutants was carried out according to the scheme in FIG. 1.

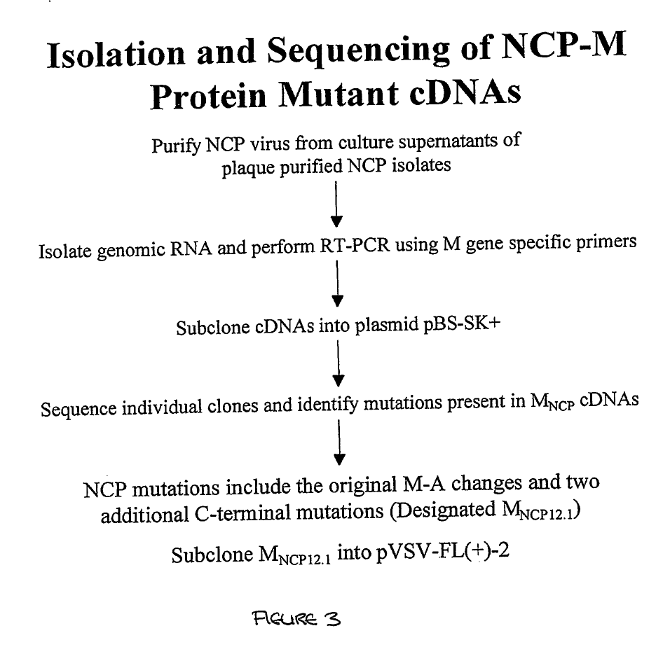

By eliminating the two translational start codons, M2 and M3 proteins were not produced and cells infected with the mutated virus showed a delay in cell rounding, a hallmark of VSV CPE (FIG. 2). The isolated recombinant virus further encoded for green fluorescent protein (GFP), which indicated which cells were infected with replicating virus. After 24 hours ˜30% of the cells did not show obvious signs of VSV. CPE. The other ˜70% of the cells, however, which were rounded, were further cultured, and upon reaching confluence were GFP-positive, indicating that the cells contained replicating virus, yet were not killed by the infection and continued to grow. To determine if the cells produced infectious virus, culture supernatants were harvested and aliquots used to infect fresh cells. After 24 hours newly infected cells demonstrated no signs of CPE, yet all expressed GFP, indicating that infectious virus was produced and could be transferred to naïve cells. Moreover, these cells also produced infectious virus and the noncytopathic (NCP) phenotype was maintained.

In order to identify which domains are involved in the M protein-induced cytopathic effects, several different M protein mutants were generated. Out of the six individual isolates obtained, all had the same mutations. The mutations resulted in the substitution of a methionine residue for an alanine residue at position 33 (SEQ ID NO: 1), and in the substitution of a methionine for an alanine residue at position 51 (SEQ ID NO: 2). Further substitutions included replacement of an alanine residue by a threonine residue at position 133 (SEQ ID NO: 3); and replacement of a glycine residue by a serine residue at position 226 (SEQ ID NO: 4) (FIG. 3).

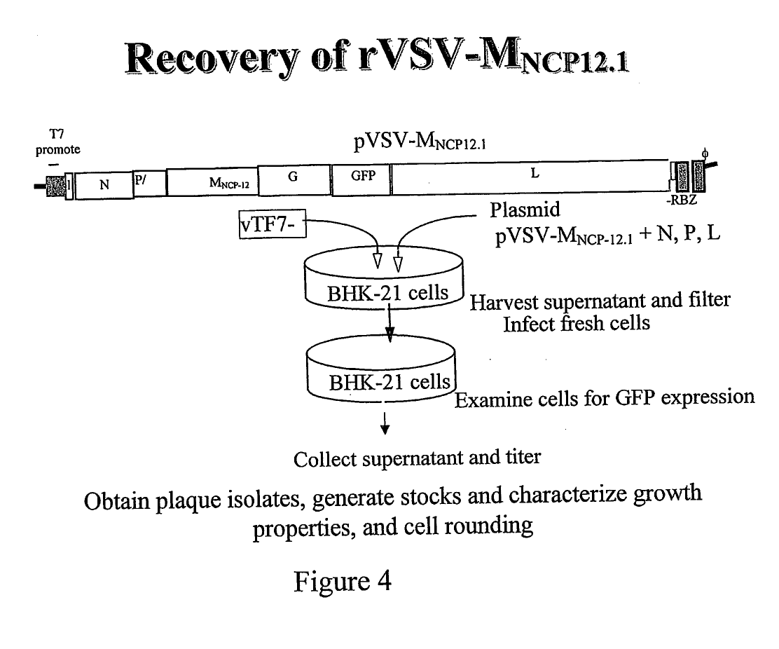

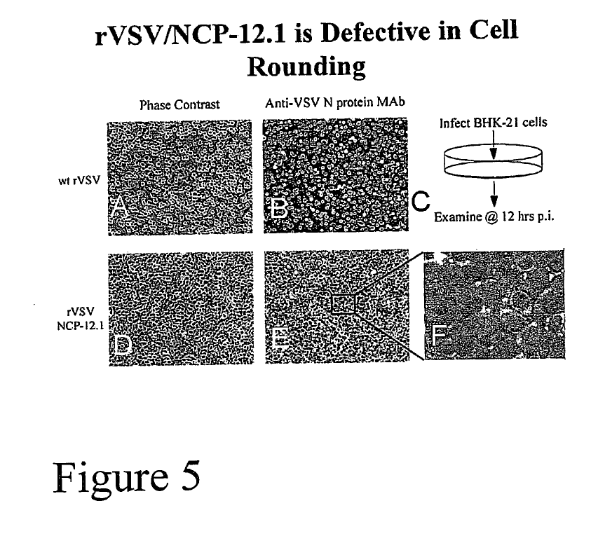

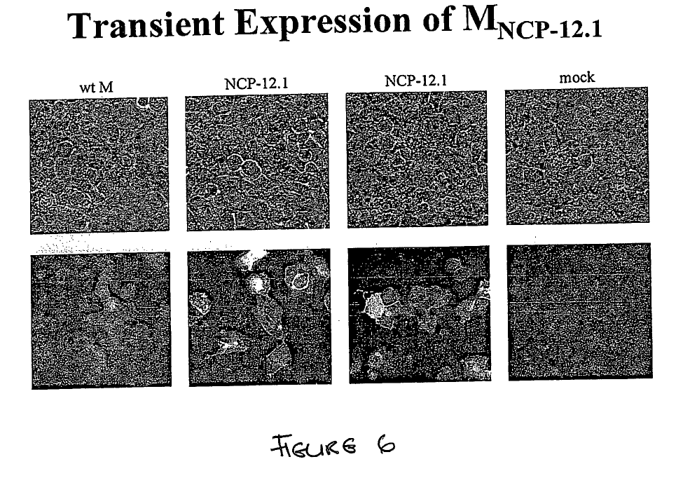

In order to determine that the NCP phenotype was due exclusively to mutations in the M protein (and not as a function of other mutations in the NCP virus genome), a cDNA was cloned for the M gene containing the four identified mutations (SEQ ID NO: 5) by replacing the wild-type M gene with the mutant gene, which was designated M_{NCP12.1}. Recombinant virus was recovered using standard procedures and the phenotype of the recovered viruses was examined (FIG. 4). The recombinant virus was infectious, yet did not mediate cellular cytopathic effects in infected BHK-21 cells (FIG. 5, B & D). Cells maintained typical morphology despite infection with the replicating mutant virus (FIG. 5C). In contrast, cells infected with wt-VSV demonstrated typical cell rounding associated with lethal VSV infection (FIG. 5A).

BHK-21 cells were also transiently transfected with VSV with wild-type M protein, or the wt, NCP-12.1 mutants. Mutant M protein expression was markedly enhanced (FIG. 6 F and G) as compared to wild-type M (FIG. 6 E), as a function of wild-type M protein synthesis inhibition via M protein interference with RNA polymerase II dependent expression. Cellular cytopathic effects mediated via wild-type M protein were evident in the cells rounding-up (FIG. 6A), while cells expressing the NCP mutant remained flat and normal in appearance (FIG. 6 B and C).

### Example 2

### Mutations in the M protein of VSV do not Affect Cellular Tropism

### Materials and Methods

The following cell types were infected with rVSV/M_{NCP12.1}: BHK, CV-1, Vero, or HeLa cells, at a multiplicity of 10. Cells were incubated at 37° C. for either 12 or 24 hours, fixed in 3% paraformaldehyde and washed twice with phosphate-buffered saline (PBS) containing 50 mM glycine. Cells were then examined for GFP expression via fluorescence miscroscopy (Zeiss Axiophot, West Germany), and morphology was assessed via phase contrast microscopy.

### Results

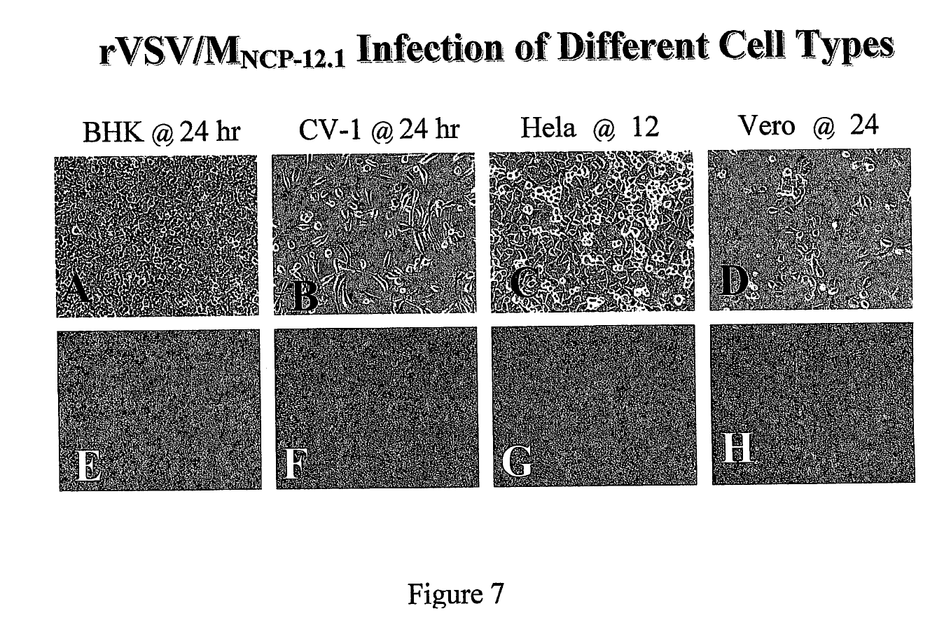

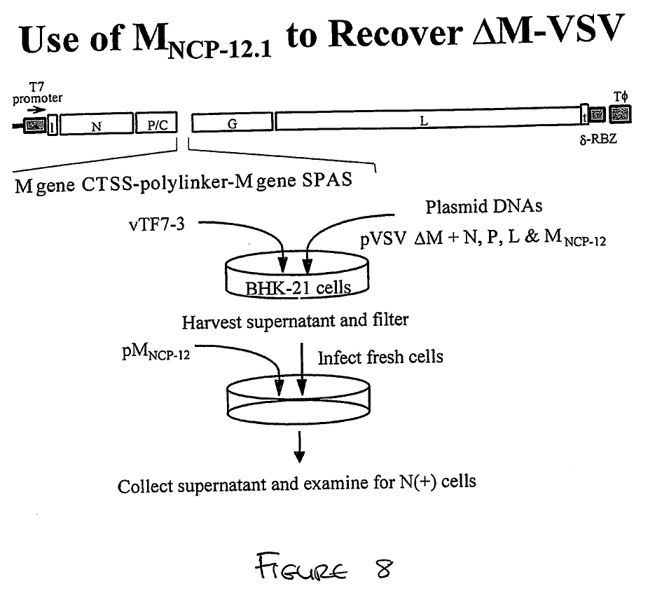

In order to determine whether noncytopathic rVSV/M_{NCP12.1} altered cellular tropism, BHK, CV-1, Vero, and HeLa cells were infected at a multiplicity of 10 (FIG. 7), and infection was determined as a function of GFP expression. Regardless of cell type, rVSV/M_{NCP12.1} was able to infect and replicate within cells, without evidence of any cytopathic effect.

### Example 3

### Development of Superior Vectors for Gene Therapy Application

### Materials and Methods

Recombinant VSV M mutants were generated as described above. Mutant virus was grown and recovered via co-expression with plasmids expressing N, P and L proteins druing BHK-21 cell infection. Mutant virus was propagated in cells expressing M_{NCP12.1}. Supernatants from cells infected with rVSV-ΔM (VSV replicon) were applied to cells transfected 24-hours prior with 5 μg of pc-M_{NCP12.1} plasmid. Cells were fixed at 24 hours post infection and probed with an N-specific monoclonal antibody labeled with a rhodamine conjugated goat anti-mouse secondary antibody (Jackson ImmunoResearch Laboratories, Inc.).

### Results

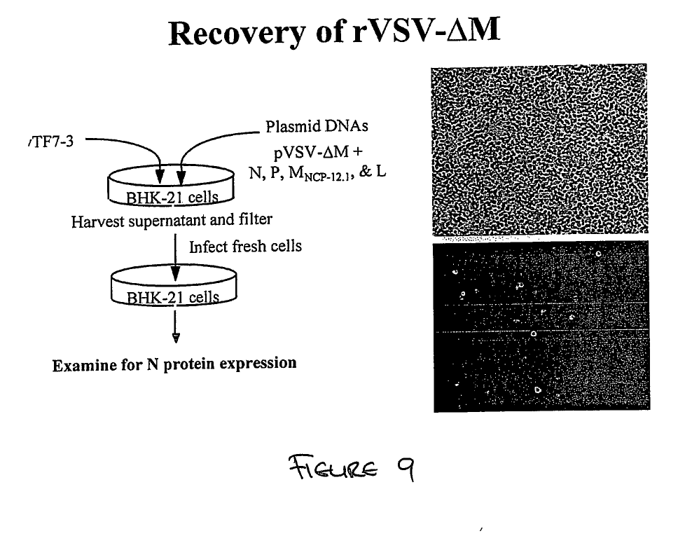

Previous attempts to recover VSV mutated or deleted for the M protein (ΔM-VSV) failed, presumably because M protein toxic effects killed the cells thereby limiting the amount of M protein expressed. In order to determine whether the methods and strains above provide a readily recoverable vector, a recovery scheme (FIG. 8) similar to the standard method used to recover recombinant VSV was utilized, with the exception that M_{NCP12.1} was co-expressed with N, P, and L support plasmids during initial viral recovery, followed by propagation of the virus from cells (or a cell line) expressing M_{NCP12.1}.

ΔM-VSV was readily recoverable under these conditions (FIG. 9), and hence is an excellent gene delivery/gene therapy vector candidate. ΔM-VSV replicated exclusively in the cell cytoplasm, eliminating potential problems of insertional mutagenesis and transgene silencing, which is often a byproduct with the use of other typical gene delivery vectors, such as retroviral gene therapy vectors.

### Example 4

### Recombinant VSV Deleted for the M and G Protein Infect Islet Cells and are not Cytopathic

### Materials and Methods

Informed consent was obtained from participating patients at the islet lab at the University of Tennessee. Islet cell preparations were prepared, infected with either a replication competent VSV deleted for the M protein (NCP12) or the replication restricted VSV, deleted for both M and G proteins (ΔG-NCP12). Cells were maintained in a volume of 750 ul media in a 12-well plate. Following infection, the viral inoculum was not removed and islets were harvested at day 3 and day 8 post infection for imaging as well as flow cytometry analysis. A small volume of the islet cells was removed from the plate and spun at 1500 rpm for 5 minutes. The pellet was resuspended in 100 ul PBS and added as a suspension on the glass plate, covered with glass coverslip and observed under 40× objective. The remainder was stained with Annexin V to determine the percent of apoptotic islet cells.

### Results

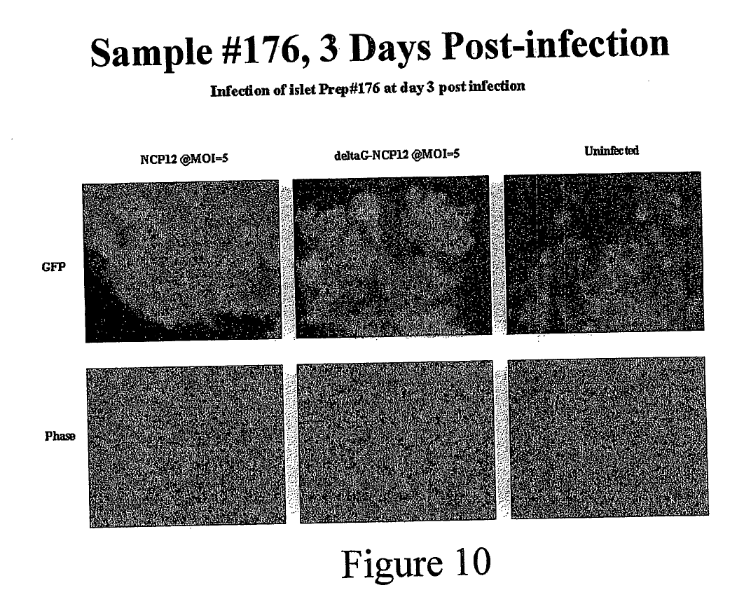

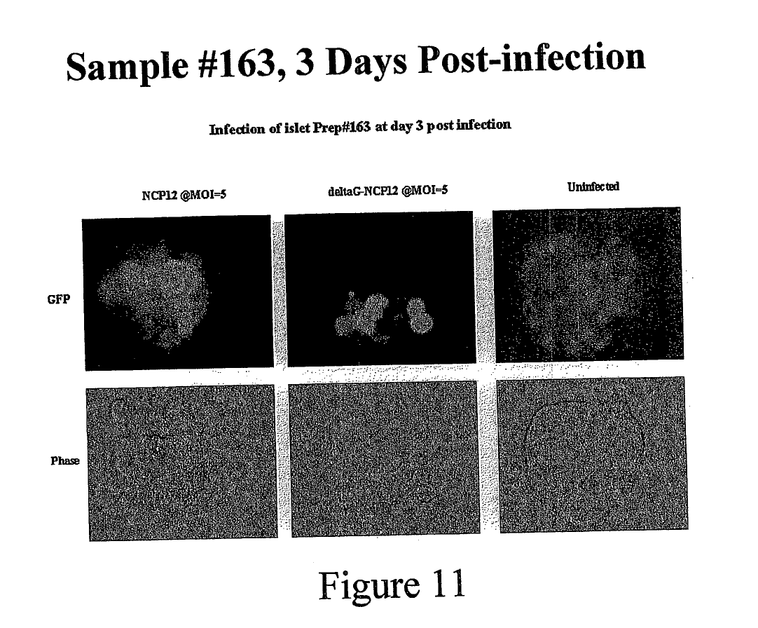

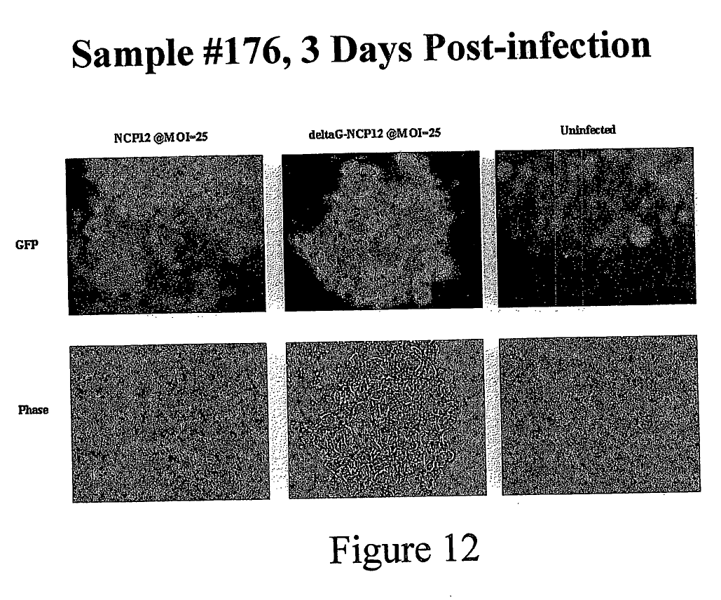

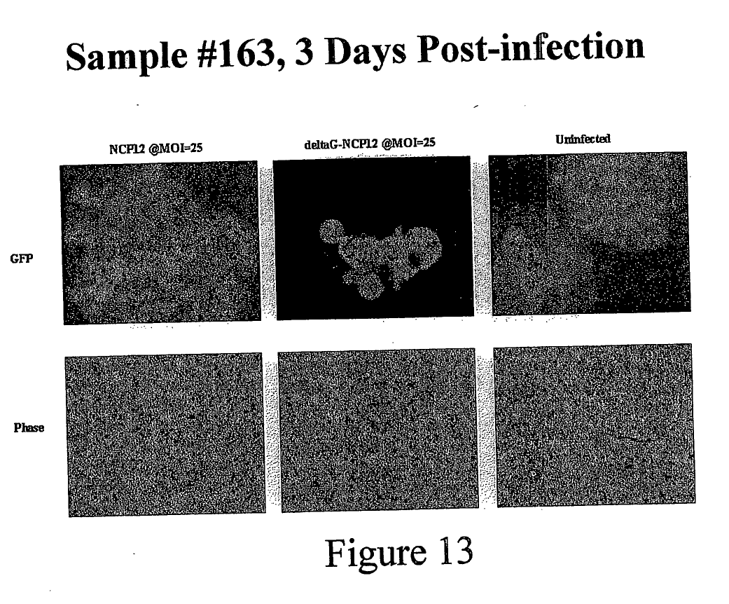

Two samples were studied, sample number 176 and 163. In both samples, infection with a recombinant VSV deleted for the M protein, at an MOI of 5, resulted in high levels of expression in infected cells (FIGS. 10 and 11 respectively) with minimal cytopathic effects. Deletion of the G glycoprotein had no obvious effect on the level of infection, however, in general sample 176, was more efficiently infected. Infection with an MOI of 25 did not produce discernable increases in either infection rate, or cytopathic effects (FIGS. 12 and 13, respectively), at 3 days post infection.

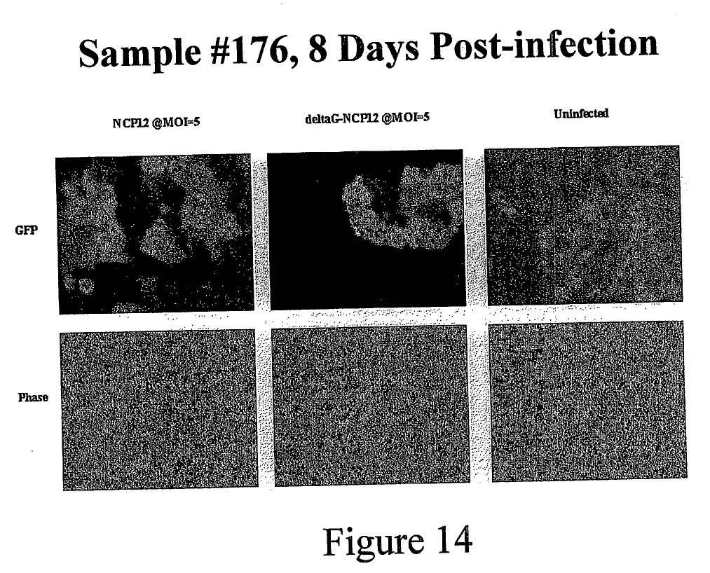

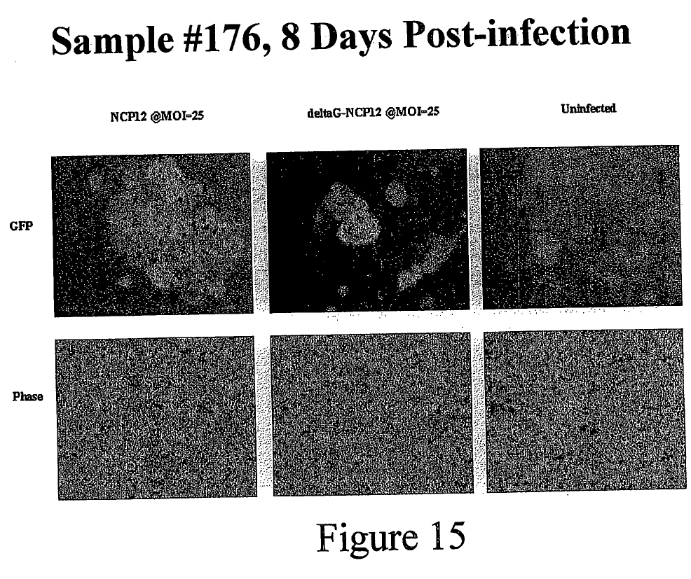

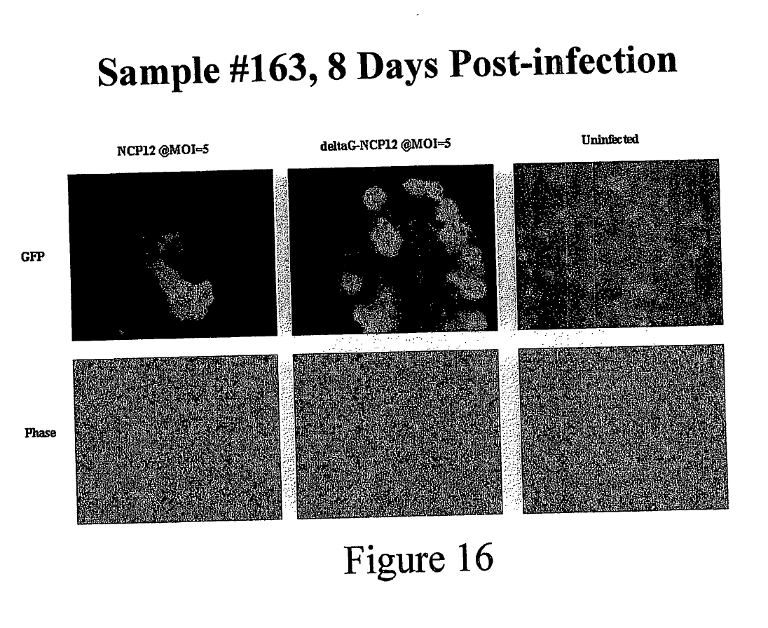

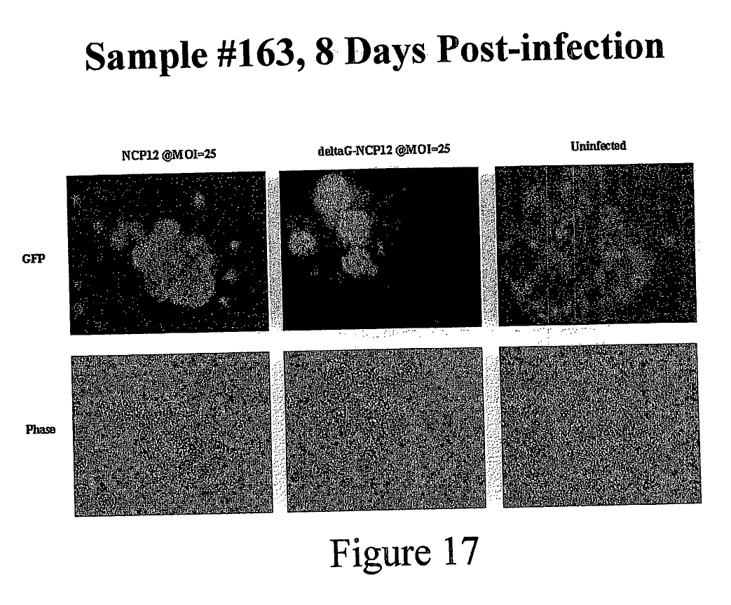

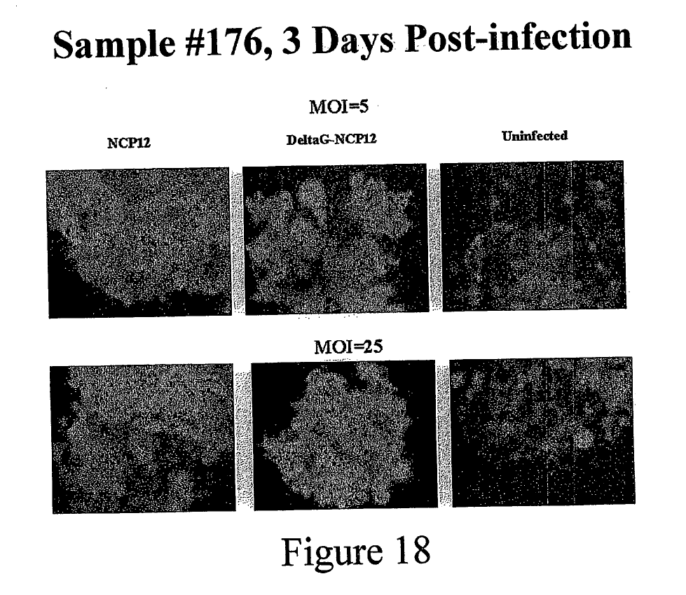

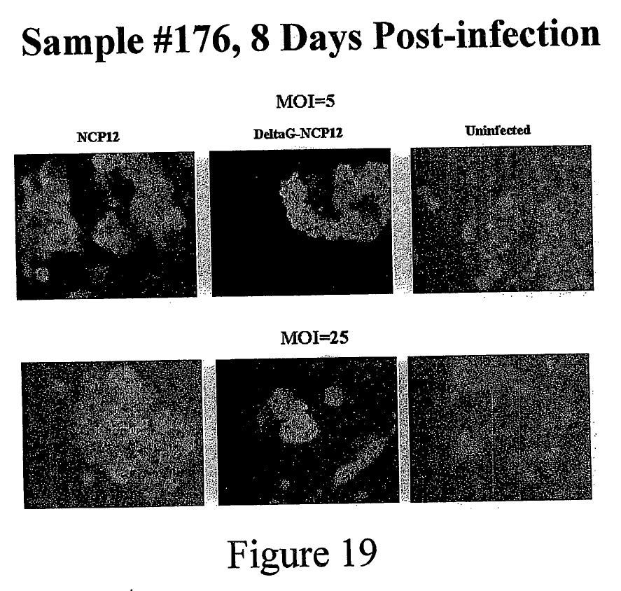

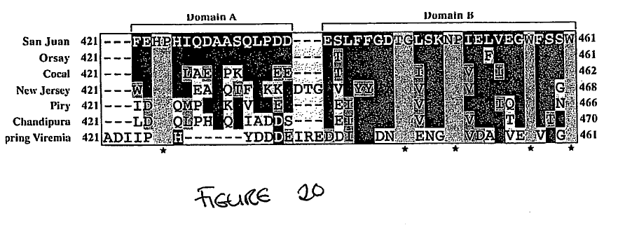

Infection persisted out to 8 days in culture, with no discernable reduction in expression, regardless of MOI (FIGS. 14-17). Regardless of MOI, high levels of expression were detected at 3 and 8 days post infection (FIGS. 18-19). Thus, islet cells are efficiently infected with the VSV constructs, with infection resulting in no discernable cytopathic effects, further emphasizing the utility of such constructs as gene delivery vehicles.

### Example 5

### Mutations in the Membrane-Proximal Region of the VSV G Ectodomain do not Affect G Protein Expression or Stability

### Materials and Methods

Plasmids and Oligonucleotide Directed Mutagenesis.

The gene encoding the G protein of VSV, serotype Indiana, strain San Juan, was cloned into the eukaryotic expression vector pXM to produce the plasmid pXM-G as described earlier (14). Mutants E452A (SEQ ID NO: 6), G456D (SEQ ID NO: 7), W457A (SEQ ID NO: 8), F458A (SEQ ID NO: 9) and W461A (SEQ ID NO: 10) were constructed by oligonucleotide-directed mutagenesis (14) and the mutated regions were cloned into pXM G(AXB) (32). The double- and triple-mutants, G456DW457A (DA) (SEQ ID NO: 11), W457AW461A (WW-AA) (SEQ ID NO: 12), W457AF458AW461A (AAA) (SEQ ID NO: 13) and G456DW457DW461A (DAA) (SEQ ID NO: 14) were generated by using a Quick Change Site-Directed Mutagenesis Kit according to the manufacturer's instructions (Stratagene, Canada). The pXM-G(AXB) plasmid was used as the template. Two complementary synthetic oligonucleotide primers (from McMaster University Central Facilities) containing the desired mutation were used for mutagenesis using turbo Pfu DNA polymerase. The deletion and insertion mutants were constructed as described earlier (32). Construct GD9 and DF440-N449 was made by deleting amino acids 453-461 (SEQ ID NO: 15) and 440-449 (SEQ ID NO: 16), of the VSV G ectodomain. Constructs GLODAF and GD9-10DAF were made by inserting 9 amino acids (residues 311-319) from the juxtamembrane region of decay acceleration factor (DAF) (7) between amino acids 464 and 465 of VSV G(AXB) (SEQ ID NO: 17) and GD9 (SEQ ID NO: 18), respectively. The reason we call these constructs “10-DAF” is because the vector G(AXB) contains additional serines at the TM junction due to insertion of a restriction site. The chimera G(+9)gBG was constructed by inserting amino acids 721-726 and 773-795 of herpes simplex virus type 1 (HSV1) glycoprotein gB between amino acid 464 of the ectodomain and amino acid 483 of the cytoplasmic tail of VSV G, such that the membrane anchoring (TM) domain of VSV G (residues 465-482) was replaced by the third TM domain of HSV1 gB protein (38). This chimera contains an extra serine residue at the ecto-TM domain junction to maintain the reading frame of VSV G protein.

The constructs W457-A (W1A), W461-A (W2A), W457W461-AA (WW-AA), G413, GSrev11, GSrev11-AA were made using an overlap PCR method with the plasmid pVSV 9.1(+) (34) as template. The sequences of the primers used are available upon request. The overlap PCR products were then purified on a 6% polyacrylamide gel, electroeluted, digested with unique restriction enzymes KpnI and NheI and used for subcloning into pVSV-FL(+)₂ (34) that was previously digested with the same enzymes. The sequences were then confirmed by dideoxynucleotide sequencing. The G genes having the desired mutations were also subcloned into a modified form of the eukaryotic expression vector pCAGGS-MCS as MluI and NheI fragments. The constructs pVSV-DAA, -AAA, -G10DAF, -G(+9)gBG, -GA9, -GA9-10DAF, and -DF440-N449 were generated by amplifying the region of the G gene between the KpnI site and the 3′ end using the corresponding pXM plasmid containing the mutant G genes as templates and subcloning this region into pVSV-FL(+)₂.

Recovery of the viruses was conducted as described (33-35) with a few modifications. Briefly, confluent monolayers of BHK-21 cells in 35 mm plates were infected with a recombinant vaccinia virus encoding the T7 RNA polymerase (vTF7-3) (36) at a multiplicity of 5 for 1 hour at 31° C. The cells were then transfected with a DNA:liposome suspension consisting of 5 mg of pVSV(+)-G* (* indicates G gene with mutations), 3 mg, 5 mg, 1 mg and 8 mg of plasmids containing the N, P, L and G genes from VSVIND respectively and 90 ml of TransfectACE (36,37). After 3 hours, the transfection mix was replaced with DMEM+10% FBS and cells were incubated at 37° C. The supernatants were collected after 48 hrs of incubation and filtered through a 0.2 m filter (Millipore, Millex-GS) to remove vaccinia virus. The filtrates were applied to BHK-21 cells that had been transfected with 2 mg of pCAGGS-GIND 24 hours earlier. Recovery of the virus was assessed by examining the cells for cytopathic effects that are typical of a VSV infection after 24-36 hours. The recovered viruses were then plaque purified, passaged and their RNA was isolated. Mutations in the G genes were confirmed by RT-PCR sequencing.

### Results

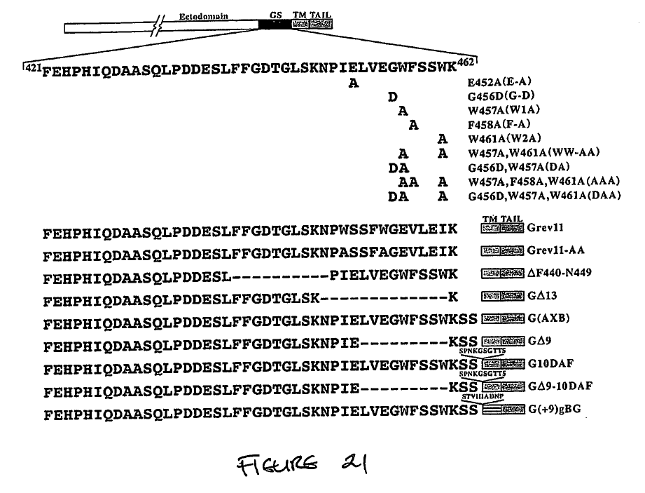

To determine which residues in the membrane-proximal region of VSV G may be important for membrane fusion activity, sequence alignments of the membrane-proximal domains from different vesiculoviruses were performed (FIG. 20). The alignment shows that this region is well-conserved across closely related vesiculoviruses and two distinct domains labeled as A and B based on the number of conserved residues in these subdomains were defined. The greatest conservation (>90%) was in the region between residues E437 and W461 (Domain B). The amino acid sequence between residues F421 and D436 (Domain A) was identical for the two strains of the VSV Indiana serotype (San Juan and Orsay), while there was >80% conservation with Cocal virus, which is a more distantly related virus classified as an Indiana-2 serotype and the other two Indiana serotype viruses. However, domain A was not well conserved among the other viruses that were examined. The tryptophan (W) residues at position 457 and 461, were conserved across all vesiculoviruses examined. Apart from the tryptophan residues, several other amino acids (H423, P424, T444, G445, N449 and P450) were also conserved across the vesiculovirus sequences examined. The FFGDTG (SEQ ID NO: 19) motif near the beginning of domain B and extending from residue 440 to 445 was conserved across most of the closely related members with the TG residues being invariant across all vesiculoviruses examined.

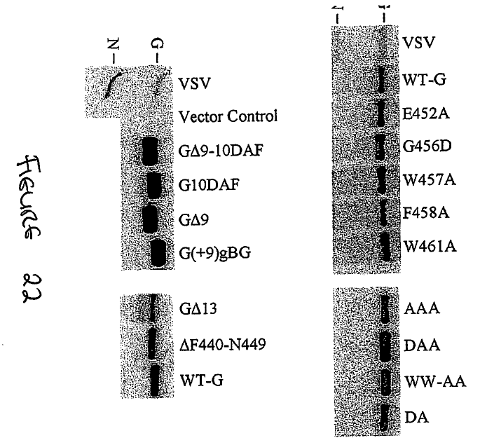

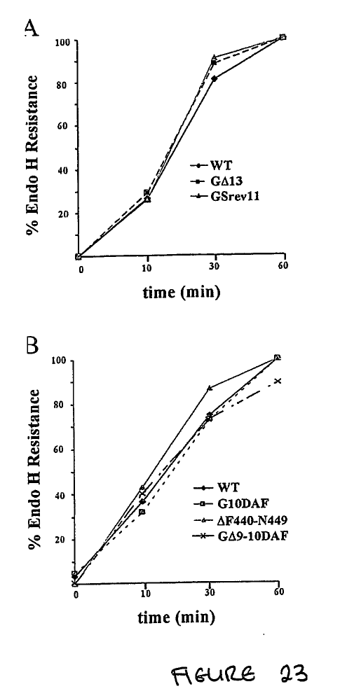

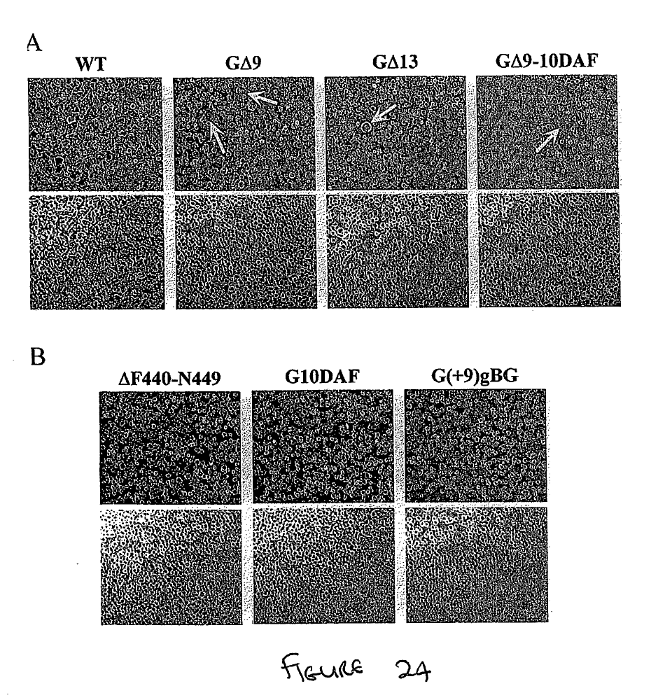

To determine the role of these conserved regions in the membrane fusion activity of G protein we made substitutions, deletions and insertions in this region (FIG. 21). The two invariant tryptophan (W) residues at position 457 and 461 were replaced with alanine. Similarly the conserved glutamic acid (E452), glycine (G456) and phenylalanine (F458) were individually replaced with alanine, or aspartic acid and alanine. Two double mutants were also constructed by replacing W457 and W461 with alanines as well as G456 and W457 with aspartic acid and alanine, respectively. In addition, two triple mutants were also generated by replacing W457, F458 and W461 with alanine and by substituting G455, W457 and W461 with aspartic acid and alanines, respectively. The mutants E452A, G456D, W457A, F458A, W461A, W457W-461-AA; G456D-W457A; G456D-W457A-W461A and W457A-F456A-W461A are also referred to as E-A, G-D, W1A, F-A, W2A, WW-AA, DA, DAA and AAA, respectively.

In addition to the above point mutations the deletion mutants (GA9, which is a deletion of amino acids 453-461 (SEQ ID NO: 15); GA13, a deletion of residues 449-461 (SEQ ID NO: 20); and AF440-N449, a deletion of amino acids 440-449) (SEQ ID NO: 16), and several insertion mutants were prepared. The construct G(AXB) introduces two additional serines between K462 and S463 {described previously in (38)}, while G10DAF and GΔ9-10DAF contain 9 aa (residues 311-319) from decay acceleration factor (DAF) inserted between aa S464 and S465 of G(AXB) and GΔ9, respectively. The mutant G(+9)gBG has an insertion of 9 residues between the ectodomain of G(AXB) and the transmembrane domain of GgB3G (39) (SEQ ID NO: 21). The remaining mutants that were examined have the sequence of the 11 aa adjacent to the transmembrane domain inverted (Gsrev11) (SEQ ID NO: 22). The mutant GSrev11-AA has the same 11 aa inverted and also has the two W residues changed to alanine (SEQ ID NO: 23).

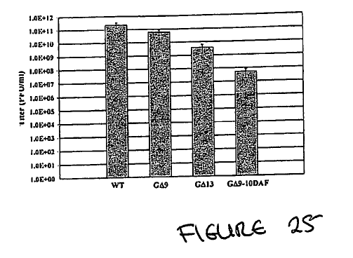

The mutant genes were expressed in COS-1 cells using the pXM vector (11), the proteins were labeled with [³⁵S]-methionine and then analyzed by immunoprecipitation with a polyclonal anti-G antibody followed by SDS-PAGE (FIG. 23). All of the substitution mutants co-migrated with the wild-type G protein and the intensities of bands corresponding to the wild-type and the mutants were similar, suggesting that the substitution of conserved residues and deletion or insertion of extra residues in the context of the G protein did not affect the expression or stability of the proteins.

Recovery of recombinant viruses encoding the mutant G proteins was accomplished via WT G protein co-expression during the initial recovery and subsequent amplification steps. This ensured that all viruses could be recovered, regardless of whether the G protein mutant was membrane fusion defective or not.

### Example 6

### Transport and Cell Surface Expression of Mutant G Proteins

### Materials and Methods

Indirect immunofluorescence assays were used to examine surface expression of the various G proteins. Cells were transfected, fixed with 3% paraformaldehyde and probed with G-specific monoclonal antibody (mAb I1) (40) followed by rhodamine conjugated goat anti-mouse (or anti-rabbit) secondary antibody (Jackson Immunoresearch Laboratories, Inc.). To quantify surface expression of G protein, flow cytometric analysis of virus infected cells or lactoperoxidase-catalyzed iodination of transfected COS-1 cells, were conducted (15). For flow cytometry, BHK-21 cells (5×10⁵) in 35 mm plates were infected with either wild type VSV or the appropriate G-complemented mutant virus at a multiplicity of 10. Six hours post-infection the cells were removed from the plates using PBS containing 50 mM EDTA and pelleted by centrifugation at 1250×g for 5 minutes. The cells were then fixed in suspension using 3% paraformaldehyde for 20 minutes at room temperature. The cells were washed two times with PBS-glycine to remove the fixative. The cells were then incubated in PBS-glycine+0.5% bovine serum albumin (BSA) (Sigma-Aldrich) for 30 minutes at room temperature. Following blocking with BSA, cells were probed with I1 mAb as primary antibody and rhodamine-conjugated goat anti-mouse antibody. The cells were then analyzed by flow cytometry to quantify surface expression levels of the various mutant G proteins.

### Results

Both cell-cell fusion and viral budding requires viral protein localization to the plasma membrane. In order to determine whether viral proteins were transported to the cell surface, both flow cytometry or lactoperoxidase-catalyzed cell surface iodination were conducted. Mutant G proteins were expressed on the cell surface at levels between 80% to >100% of wild-type G protein (Table 1).

TABLE 1
Surface expression and membrane fusion activity of mutant G proteins.

| | Relative | | | |
|---|---|---|---|---|
| | Surface Expression | | Percent (%) | |
| | Surface | | Fusion Activity | |
| | Iodina- | Flow | Transfected | Infected |
| Expressed Protein | tion^{a} | Cytometry^{b} | cells^{c} | cells^{d} |
| WT | 1.0 | 1.0 | 100 | 100 |
| E452A (E-A) | 1.17 | N.D. | 50 | N.D. |
| G456D (G-D) | 0.82 | N.D. | 30 | N.D. |
| W457A (W1A) | 1.11 | 0.91 | 70 | 100 |
| F458A (F-A) | 1.25 | N.D. | 35 | N.D. |
| W461A (W2A) | 1.64 | 1.04 | 30 | 100 |
| WW-AA | 1.50 | 0.95 | 15 | 100 |
| G456DW457A (DA) | 1.35 | N.D. | 30 | N.D. |
| DAA | 1.85 | 1.03 | 5 | 100 |
| AAA | 1.80 | 1.15 | 5 | 100 |
| G(AXB) | 1.0 | 0.98 | 100 | 100 |
| GSrev11 | N.D. | 0.97 | N.D. | 100 |
| GSrev11-AA | N.D. | 1.01 | N.D. | 100 |
| GΔ9 | 2.08 | 1.01 | <1 | <1 |
| GΔ13 | 1.15 | 1.1 | <1 | <1 |
| ΔF440-N449 | 1.10 | 1.01 | <1 | 0 |
| G10DAF | 1.28 | 1.06 | <1 | 0 |
| GΔ9-10DAF | 2.70 | 1.16 | <1 | <1 |
| G(+9)gBG | 1.2 | 1.03 | <1 | 0 |

^{a}COS cells were transfected with pXM vectors encoding the indicated G protein, the cells were surface iodinated and the relative amount of surface expression was calculated as described previously (15).
^{b}BHK-21 cells were infected with G-complemented viruses and fixed at 6 h post-infection. The cells were then stained with the G-specific monoclonal antibody I1 and a rhodamine-labeled secondary antibody, and then analyzed by flow cytometry. Relative surface expression was calculated using the following formula: (% positive cells in mutant population × mean fluorescence intensity of mutant)/(% WT positive cells × mean fluorescence intensity of WT).
^{c}Transfected COS cells were bathed in fusion medium buffered to pH 5.6 and cell-cell fusion was determined as described in the Materials and Methods.
^{d}BHK-21 cells were infected with the respective virus mutants and incubated in fusion medium buffered to pH 5.9 as described. Values are expressed as a percentage of WT fusion activity, which was set at 100%.
N.D. = not done.

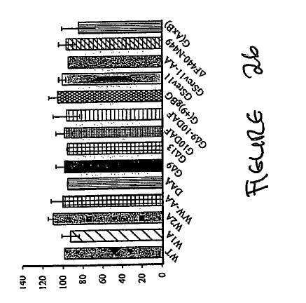

To determine whether mutations in the membrane-proximal region affected the intracellular transport or cell surface localization of G protein, wild-type and mutant G proteins in both COS-1 and BHK-21 cells were expressed. Transport from the endoplasmic reticulum (ER) to the Golgi complex was evaluated by examining the acquisition of endo H resistance. After a 15-nin pulse all the mutants were sensitive to endo H, demonstrating that they were glycosylated with N-linked oligosaccharides. Following a 1-hour chase, the mutants were resistant to endo H digestion, indicating that all the mutant G proteins were transported from the ER to the Golgi with similar kinetics. Representative examples of some mutants expressed in BHK-21 cells are shown in FIG. 24.

### Example 7

### G Protein Deletion Mutants are Reduced in Their Capacity to Promote Membrane Fusion

### Materials and Methods

Virus-infected BHK-21 cells and plasmid-transfected COS-1 cells were utilized for assays determining syncytia formation. Media was removed at six hours post-infection, in virus infected cells, then cells were rinsed once with fusion medium [10 mM Na2HPO4, 10 mM N-2-hydroxyethylpiperazine-N′-2-ethanesulfonic acid (HEPES), 10 mM 2-(N-morpholino)ethanesulfonic acid (ES)] titrated to the indicated pH (5.9, 5.5 or 5.2) with HCl] and bathed for 1 minute in fresh fusion medium at room temperature. After 1 minute, fusion medium was replaced with fresh Dulbecco's modified Eagle's medium (DMEM) containing 5% fetal bovine serum (FBS) and cells were incubated at 37° C. for 30 minutes. Cells were then fixed with 3% paraformaldehyde and processed for immunofluorescence as described above. Transfected COS-1 cells were processed as previously described (11).

### Results

To examine the effect of the mutations on membrane fusion activity, a cell-cell fusion assay was conducted, in which cells expressing either WT or the respective G mutants were treated with fusion medium buffered to pH 5.9 to 4.8. When assayed in transiently transfected COS-1 cells all of the substitution mutants showed reduced membrane fusion activity ranging from 70% to 5% of wild-type activity (Table 1).

However, when assayed in virus-infected BHK cells, many of these mutants (W1A, W2A, WW-AA, DAA, and AAA) produced extensive syncytia similar in size and extent to that seen in wild-type VSV infected cells. The basis for this difference is not fully understood, but it may simply be a function of the level of G protein expression in virus infected versus transiently transfected cells.

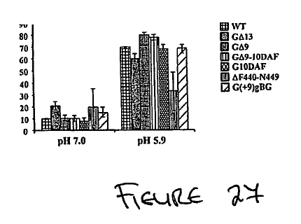

In contrast to the substitution mutants, the deletion and insertion mutants had very low to undetectable membrane fusion activities in both COS and virus-infected BHK cells. The mutants GΔ9, GΔ13 and GΔ9-10DAF produced very few syncytia that had only three to four nuclei when cells were exposed to pH 5.9 (FIG. 25A, arrows). When cells expressing these proteins were bathed in medium buffered to pH 5.5, 5.2, or 4.8 neither the size nor the number of syncytia increased (data not shown), indicating that the defect in syncytia formation was not due to a shift in the pH threshold. In addition, prolonged incubation following exposure to the low pH trigger did not increase the number or size of syncytia seen (data not shown). When 9 or 10 amino acids were inserted between the boundary of the membrane anchoring domain and the ectodomain {mutants G(+9)gB and G10DAF)}, and when residues F440-N449 were deleted, there was a complete loss of membrane fusion activity (FIG. 25B). These data suggest that the sequence context of the juxtamembrane region is critical for fusion activity. In all of these cases increasing the time of exposure to acidic pH or decreasing the pH of the fusion medium did not enhance membrane fusion activity.

Conserved membrane-proximal aromatic residues (tryptophan and phenylalanine) and the glycine residue were therefore not critical for VSV G induced membrane fusion in the context of virus infected cells, but when expressed transiently in COS-1 cells, some reduction in activity was seen. Inversion of the 11 membrane-proximal residue sequence had no significant influence on the fusion activity, as well. While the linear order of the 9 membrane-proximal amino acids did not seem to affect membrane fusion activity, these residues are critical, since their deletion reduced fusion activity by >99%. Also, deletion of 13 amino acids had a similar effect on cell-cell fusion mediated by G protein. However, deletion of the region between F440 and N449 which includes the conserved FFGDTG (SEQ ID NO:) motif completely abolished fusion activity showing that this sub-domain is important for the fusion activity of G. Results with the insertion mutants G10DAF and G(+9)gBG showed that although these insertions do not affect surface expression, they completely abolished fusion activity indicating that the spacing of the membrane-proximal domain from the transmembrane domain is very important for membrane fusion activity. In accordance with this concept, when the length of the juxtamembrane region in G10DAF was reduced by deleting the 9 membrane-proximal residues (GD9-10DAF), membrane fusion activity was partially restored. Taken together these results indicated that the region immediately adjacent to the membrane-anchoring domain is essential for the membrane fusion activity of VSV G protein, and unlike, for example, in HIV-1 gp41 (41) the conserved membrane-proximal W residues in VSV G are not critical for G protein incorporation into virus or for membrane fusion activity.

### Example 8

### Stability of the Mutant G Proteins

### Materials and Methods

The oligomeric state of the expressed G proteins was determined by sucrose density gradient centrifugation as described (15, 17). The endoglycosidase H assays were performed on transfected cell lysates, as described (15) and with virus-infected cell lysates, as described (10), with a few modifications. BHK-21 cells grown in 35 mm plates were infected with the appropriate G-complemented mutant viruses at a multiplicity of 10. Six hours post-infection the cells were rinsed once with methionine-free DMEM (Met-free DMEM) and then incubated in 2 ml Met-free DMEM for 20 minutes. The media was replaced with 2 ml of Met-free DMEM containing 55 mCi of [³⁵S] methionine (Translabel protein labeling mix, New England Nuclear) for the indicated amounts of time. Following the pulse period the cells were either immediately lysed with 1 ml of detergent lysis buffer [10 mM Tris (pH 7.4), 66 mM EDTA, 1% TX-100, 0.4% deoxycholic acid, 0.02% sodium azide] or chased with DMEM+10% FBS medium containing 2 mM excess non-radioactive methionine. Nuclei and cell debris were removed by centrifugation at 14,000 rpm for 1 minute on a tabletop micro-centrifuge (IEC Centra). Immunoprecipitation was performed with anti-G tail rabbit polyclonal antibody (peptide Ab #3226) essentially as described previously (42) except that the post-nuclear supernatants were made to 0.3% sodium dodecyl sulfate (SDS) and the antigen-antibody complexes were formed for 1 hour at 37° C. One half of the immunoprecipitates were digested with Endo H (New England Biolabs) according to the manufacturer's instructions.

Trypsin sensitivities of the wild-type and mutant proteins were determined as described (38,43). Briefly, transfected cells were labeled with [³⁵S]methionine and lysed with 1% Triton X-100 in 2×MNT [40 mM 2-(N-morpholine)ethanesulfonic acid, 60 mM Tris, 200 mM NaCl, 2.5 mM EDTA] buffer at the indicated pH. The lysate was centrifuged at 14,000 g for 5 minutes, and equivalent volumes of the supernatant incubated in the absence or the presence of 10 mg TPCK-trypsin for 30 minutes at 37° C. The digestion was stopped by addition of aprotinin (100 units), and the mixture was centrifuged again at 14,000 rpm for 2-5 min to remove any insoluble material. The supernatant was immunoprecipitated with anti-G (Indiana) antibody and analyzed by SDS-PAGE.

Titers of the recovered viruses were determined by plaque assay. BHK-21 cells (5×10⁵) in a 6 well plate were infected with 10-fold serial dilutions of the virus. At one hr post infection the inoculum was removed and the cells were overlayered with DMEM containing 0.9% agar and 5% FBS, and incubated at 37° C. for 36 hours. After the incubation period the number of plaques were counted and averaged between at least two dilutions. Virus titers were expressed as plaque forming units (PFU)/ml. The plaques were photographed with a Nikon digital camera using a 75-200 Nikorr lens. The digital images were then magnified and printed on a 4×8 inch Kodak photo paper. The sizes of at least 15 plaques per virus were determined and averaged.

### Results

Although all of the mutant proteins were expressed on the cell surface, indicating they could fold and oligomerize sufficiently in the ER to be transported to the plasma membrane, we have shown previously that some mutations can affect trimer stability in sucrose density gradients without affecting transport (43,44). To determine whether the mutations in the membrane-proximal region that reduced membrane fusion activity affected trimer stability, we utilized sucrose density gradient centrifugation at acidic and neutral pH. For the mutants that were examined, all showed sedimentation patterns that were similar to those of wild-type G with the exception of G(+9)gBG, which was slightly less stable to centrifugation in gradients buffered to pH 5.6 (Table 2).

TABLE 2
Oligomerization and Trypsin sensitivity of G protein mutants

| | Trimer Stability | Trypsin resistance | | |
|---|---|---|---|---|
| Protein | at pH 5.6 | pH 7.4 | pH 6.5 | pH 5.6 |
| WT | + | − | ++ | +++ |
| W1A | + | − | ++ | +++ |
| W2A | + | − | ++ | +++ |
| WW-AA | + | − | ++ | +++ |
| DAA | + | − | ++ | +++ |
| G(AXB) | + | − | ++ | +++ |
| GSrev11 | + | − | ++ | +++ |
| GSrev11-AA | + | − | ++ | +++ |
| GΔ9 | + | − | + | +++ |
| GΔ13 | + | − | ++ | +++ |
| ΔF440-N449 | + | − | ++ | +++ |
| G10DAF | + | − | + | +++ |
| GΔ9-10DAF | + | − | ++ | +++ |
| G(+9)gBG | +/− | − | − | ++ |
| Confluent monolayers of BHK-21 cells were infected with either WT or mutant viruses at a multiplicity of 10. Six hrs post-infection the cells were radioactively labeled and chased as described in the Materials and Methods. For the trimer assays the cells were lysed in 2x MNT buffered to pH 5.6. Lysates were then centrifuged through a 5-20% sucrose density gradient buffered to the same pH. Fractions were collected from the bottom and G proteins | | | | |
| # were immunoprecipitated with a polyclonal anti-VSV antiserum. For the trysin sensitivity assays cells were lysed in 1x MNT buffered to the indicated pH. Lysates were clarified by centrifugation to remove cell debris and nuclei. The supernatants were then treated with or without TPCK-trypsin and the G proteins were immunoprecipitated with a polyclonal anti-VSV antibody. Immunoprecipitated proteins were resolved by SDS-PAGE, visualized | | | | |
| # by fluorography and quantified by scanning densitometry. | | | | |

(+++) = 95% to 100% resistant;
(++) = 50% to 95% resistant;
(+) = 10% to 50% resistant;
(−) = <10% resistant.

Since G protein becomes resistant to trypsin digestion at low pH, presumably due to conformational changes induced by acidic pH (42), it was important to determine whether changes in fusogenic activity of the membrane-proximal mutants were due to low pH-induced conformational changes. we examined the pH dependent resistance to trypsin digestion of mutant and wild-type G proteins (Table 2). Most of the mutants showed trypsin resistance profiles similar to that observed for wild-type G protein. For example, at pH 6.5 approximately 75-80% of the mutant and wild-type G proteins were resistant to trypsin digestion. Two of the mutants, GD9 and G10DAF, were somewhat less resistant (48% and 40%, respectively) to trypsin digestion at pH 6.5, whereas the mutant G(+9)gBG showed a drastic change in the resistance pattern and was completely sensitive to digestion at pH 6.5 and only partially resistant at pH 5.6.

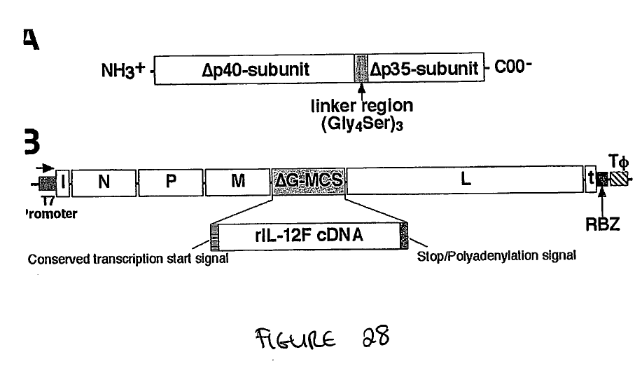

Based on the cell-cell fusion assay results we predicted that recombinant virus encoding the mutants which exhibited wild-type G fusion activity would grow similar to wild-type VSV, and those that had undetectable membrane fusion activity would not be able to grow without complementation using wild-type G. As predicted, all of the point mutants, the insertion mutant G(AXB), and the sequence reversal mutants GSrev11 and GSrev11-AA produced titers similar to wild-type VSV when grown on BHK-21 cells. However, to our surprise some of the viruses that showed a >99% reduction in cell-cell fusion activity (e.g. GΔ9, GΔ13 and GΔ9-10DAF) were able to grow and spread on BHK-21 cells without the need for expression of G protein for complementation, albeit to lower titers (FIG. 26). The deletion mutant GΔ9 gave titers that were consistently 10-fold lower than wild-type virus, while the deletion mutant GΔ13 had titers that were approximately 100-fold lower. The titer of the GΔ9-10DAF was 10,000 fold lower than that of wild-type VSV. In accordance with the reduced viral titers, plaque formation by these mutants required 48-60 hours, whereas wild-type VSV and the other mutants produced plaques by 24-30 hours post-infection (Table 3).

TABLE 3
Growth characteristics of mutant G viruses

| | Average | Relative | | | |
|---|---|---|---|---|---|
| | plaque | plaque | | Budding | |
| | size | size | Titer | efficiency | Specific |
| Virus | (cms) | (%) | (PFU/ml) | (%) | infectivity* |
| WT | 0.86 | 100 | 2.8 × 10¹¹ | 100 | 2.8 × 10¹¹ |
| GΔ9 | 0.46 | 54 | 4.4 × 10¹⁰ | 78 | 5.6 × 10¹⁰ |
| GΔ13 | 0.31 | 36.5 | 3.5 × 10⁹ | 79 | 4.4 × 10⁹ |
| GΔ9-10DAF | 0.11 | 13.6 | 4.75 × 10⁷ | 62 | 7.7 × 10⁷ |

To determine if any compensating mutations occurred in the G genes of GΔ9, GΔ13 or GΔ9-10DAF during recovery or subsequent amplification that might explain the ability to grow in the apparent absence of detectable membrane fusion activity we performed RT-PCR sequencing on the entire G gene of these viruses. No other mutations other than those specifically introduced were detected, which indicated that the phenotypes of the viruses were due to the designed mutations.

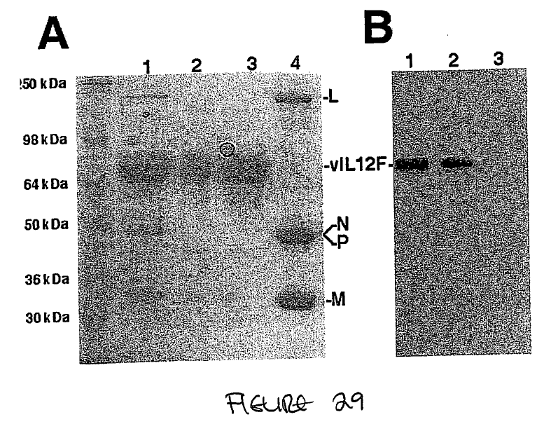

The remaining mutants, which included GLODAF, G(+9)gBG and ΔF440-N449, were noninfectious and unable to grow in BHK-21 cells without co-expression of wild-type G protein, which is consistent with their lack of cell-cell fusion activity. The lack of infectivity was not due to differences in the amount of G protein incorporated into virions since all the mutant G proteins were incorporated at levels similar to that of the WT protein (FIG. 27).

### Example 8

### Viral Binding to Cells

### Materials and Methods

A budding assay was conducted, essentially as described (46). Confluent BHK-21 cells in 35 mm plates were infected with the respective mutant viruses at a multiplicity of 10. Following adsorption, the residual innoculum was removed by rinsing the plate twice with serum-free DMEM (SF-DMEM) and washed two times in SF-DMEM with rocking at 37° C. for 5 minutes each. The cells were then incubated at 37° C. in 2 ml of SF-DMEM. Following 16 hours incubation, the supernatants were harvested and clarified by centrifugation at 1,250×g for 10 minutes. An aliquot of the supernatant was used to determine the titers of the viruses by a plaque assay. Virions were pelleted from the remaining 1.5 ml of the supernatants through a 20% sucrose cushion at 45,000 rpm for 35 minutes. The viral pellet was resuspended in 50 ml of reducing sample buffer. One-fifth of each sample (10 ml) was resolved by electrophoresis on a 10% polyacrylamide gel. The gels were stained with Coomassie (GELCODE-blue, PIERCE Co.) as per the manufacturer's instructions. The gels were destained and photographed using a Nikon digital camera using a 35-80 mm Nikkor lens. Quantification of viral protein was done using the ImageQuant analytical software (Molecular Dynamics). Virus yield was determined by measuring the intensity of the N protein band.

### Results

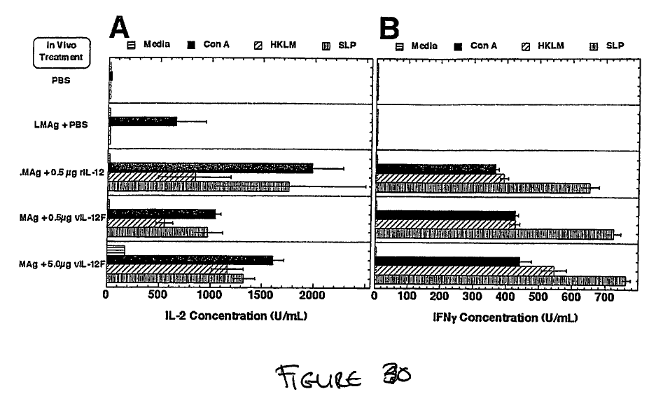

One explanation for the reduction or lack of membrane fusion activity, as well as the loss of virus infectivity in some of the mutants is that the mutations may have affected the ability of G protein to bind to cells. To determine if any of the mutations affected viral binding, radiolabeled virions were incubated with BHK-21 cells in binding media buffered to pH 7.0 or pH 5.9. Binding was conducted on ice to prevent endocytosis of the virions as well as to prevent fusion of the viral envelope with the cell membrane following exposure to low pH. VSV binding is enhanced at acidic pH (8.47). All mutants examined, except for GΔ13 and ΔF440-N449, bound to cells similar to wild-type VSV at both pHs 7.0 and pH 5.9 (FIG. 28). Both GΔ13 and ΔF440-N449 consistently gave 2-fold better binding at pH 7.0 compared to the WT; however at pH 5.9 GΔ13 binding was approximately 10% less than wild-type VSV while ΔF440-N449 binding was reduced by 50% compared to the wild-type virus (FIG. 28). These data indicate that residues in the region between F440 and V454 contribute to viral binding and that the reduced amount of binding may be partially responsible for the defect in membrane fusion activity seen with these mutants. Insertion of 10 aa from DAF (G10DAF, GΔ9-10DAF) or 9 aa from HSV gB did not affect binding indicating that the spacing of the residues between the TM and the ectodomain is not critical for binding. Based on these results it appears the defect in fusion activity and virus infectivity for G10DAF, G(+9)gBG and GΔ9-10DAF is most likely at a post-binding step.

In addition to affecting membrane fusion activity, the mutations may also reduce the amount of virus released from cells. To determine if some of the reduction in viral titers observed with GΔ9, GΔ13 or GΔ9-10DAF was due to reduced virus budding we determined the specific infectivity of each virus, which is calculated as the ratio of the virus titer to the relative amount of virus released compared to the WT virus. All three of the mutants that gave reduced viral titers produced between 60 and 90% of the amount of virus made from WT infected cells. Therefore, the defect in the ability of these mutants to spread in culture is primarily due to defects in membrane fusion activity rather than in viral budding.

### Example 9

### Recombinant VSV-ΔG Expressing IL-12F

### Materials and Methods

The following plasmids were used to produce recombinant VSV-ΔG via reverse genetics: pBS-N, pBS-P, pBS-L, and pBS-G (pBluescipt-based plasmids that encode the indicated VSV protein (48)). Plasmid pVSVΔG-PL is a Bluescript-based plasmid that expresses the anti-genome RNA of VSV-ΔG, in which the coding region for G protein has been replaced with a polylinker (49). Plasmid pCAGGS-GIND is a pCAGGS-based plasmid that encodes the Indiana serotype of VSV G protein (GIND). The IL-12F construct was obtained from Dr. Richard Mulligan (Harvard University) as a component of pSFG-mIL12.p40.L.Δ.p35 (50). The IL-12F construct was removed from the parent plasmid and cloned into Bluescript SK (Strategene, La Jolla, Calif.) as a SmaI/SmaI fragment, and was subsequently cloned into pVSVΔG-PL as a XhoI/EagI fragment to produce pVSVΔG-IL12F.

Recovery of recombinant VSV-ΔG expressing the IL-12F protein was performed using a reverse genetic strategy that was described previously [Takada, 1997 #1055; Robison, 2000 #1057]. This recovery system is based upon synthesis of recombinant anti-genomic RNA in conjunction with expression of the structural proteins of VSV in suitable host cells. Genes encoding each of the required structural proteins of VSV (N, P, L, and G) and the recombinant anti-genomic sequences are encoded on separate Bluescript-based plasmids under the control of a T7 promoter; the recombinant anti-genome plasmid is designated pVSVΔG-IL12F. Briefly, BHK-21 cells (previously infected with recombinant vaccinia virus expressing T7 polymerase; vTF7-3) were co-transfected with Bluescript-based plasmids encoding the N, P, L, and G proteins of VSV, as well as the plasmid encoding the anti-genome (pVSVΔG-IL12F), at a ratio of 3:5:1:8:5, respectively. The transfected cells were incubated for 5 hours in serum-free DMEM (DMEM-0), and then for 48 hours in DMEM supplemented with 10% FCS (DMEM-10). Culture supernatants were collected, filtered (0.2 μm) to remove vaccinia virions, and then overlayed onto fresh BHK-21 cells that had been previously transfected with pCAGGS-GIND. Because the recombinant virus does not produce G protein, it must be supplied in trans so that newly-budding virions are infectious. Recombinant virus was then plaque-purified, amplified and titered on G-complemented BHK-21 cells.

BHK-21 cells were infected with G-complemented VSVΔG-IL12F (MOI=5) and cultured in DMEM-0 for 17 h at 37° C. Culture supernatant containing IL-12F protein was collected and centrifuged at 100,000×g over 20% sucrose to remove ΔG virions. The clarified supernatant was dialyzed against three changes of sterile PBS, filter sterilized, and stored at −85° C.

Protein components of VSVΔG-IL-12F-infected BHK cell culture supernatants, as well as purified virus preparations, were separated by SDS-PAGE (10%) using a procedure modified from that previously described (51). Separated proteins were visualized by staining with Coomassie Brilliant Blue (Sigma Chemical Co., St. Louis, Mo.).

Western analysis was performed using a modified procedure from that described previously (52). Briefly, proteins separated via 10% SDS-PAGE were electrophoretically transferred to nitrocellulose membrane, which was then probed with an IL-12-specific mAb (C17.8.20.15). After several washes with TBST, the membrane was probed with anti-rat Ig peroxidase (Sigma Chemical Co). Unbound secondary Ab was removed by washing with TBST, and secondary antibody bound to the membrane was detected using the Renaissance Western Blot Chemiluminescence reagent system (NEN Life Science Products, Inc. Boston, Mass.)

### Results

Using a reverse genetics approach, we constructed a replication-restricted vesicular stomatitis virus (VSV-ΔG) that expresses large quantities of vIL-12F during its replicative cycle. The cDNA encoding IL-12F was originally constructed by Dr. Richard Mulligan and colleagues (50) as diagrammed in FIG. 28A. Production of recombinant VSVΔG-IL12F was accomplished by co-transfection of recombinant vaccinia virus (expressing T7)-infected BHK cells with plasmids encoding the VSV N, P, G, and L proteins (all under T7 promoter control) as well as a plasmid encoding the recombinant VSV anti-genome. FIG. 28B shows the organization of the recombinant VSVΔG-IL12F in which the G coding region of the anti-genome plasmid had been replaced with the IL-12F coding region. Recombinant VSVΔG-IL12F recovered from the co-transfected BHK cells was plaque-purified, amplified, and titered on BHK cells expressing G protein. The resulting G-complemented virus can infect cells and replicate, but produces non-infectious “bald” virions when G protein is not provided in trans.

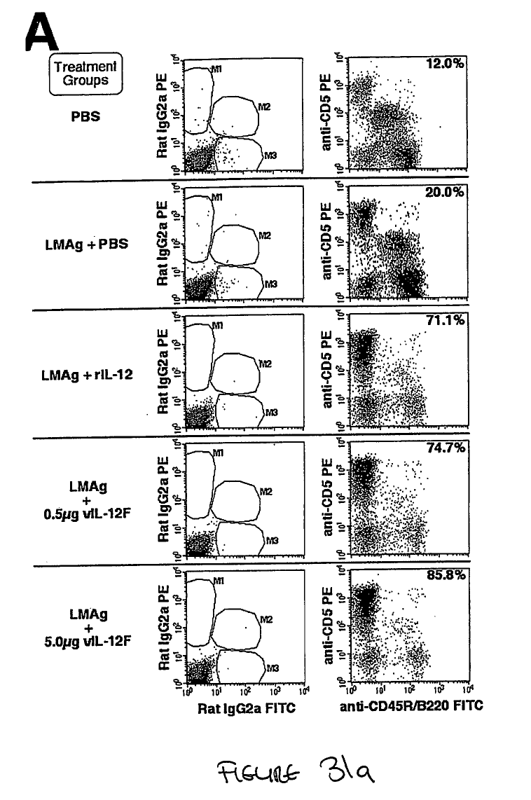

To produce vIL-12F, BHK cells were infected with VSVΔG-IL12F (MOI=5) and cultured for 17 hours in protein-free medium, and then culture supernatants were collected and clarified by centrifugation (virions were pelleted through a 20% sucrose cushion). To assess vIL-12F secretion from infected BHK cells, pre- and post-clarified supernatants and a sample of the virus pellet were analyzed by SDS-PAGE followed by staining with Coomassie Blue (FIG. 29A) and Western blot analysis with an IL-12 p40-specific mAb (FIG. 29B). The pre-clarified supernatant (FIG. 29A, lane 1) from cells infected with VSVΔG-IL-12F reveals the presence of all VSV proteins except G protein, as well as an additional band (a doublet) that corresponded to the expected size of the vIL-12F (approx. 70 kDa). Western analysis confirmed that this prominent 70 kDa band was indeed the vIL-12F (FIG. 29B, lane 1). Once the supernatant was cleared of virus (FIG. 29A, lanes 2 and 3; FIG. 29B, lane 2), the only detectable protein remaining in the clarified supernatant was the IL-12F. Analysis of the viral pellet revealed that each of the viral proteins encoded by the recombinant VSVΔG-IL12F genome (L, N and P, and M) are easily detectable (FIG. 2A, lane 4), but there is no detectable quantity of vIL-12F (FIG. 29A, lane 4 and FIG. 29B, lane 3). It should also be pointed out that, as expected, no band corresponding to the VSV G protein (the surface glycoprotein that is required for viral infectivity) was detectable. These results clearly showed that large quantities of vIL-12F were produced and secreted from VSVΔG-IL12F-infected BHK cells, and that >95% of the protein content of the clarified culture supernatants was vIL-12F.

### Example 10

### Recombinant VSV-ΔG Expressing IL-12F Enhances Antigen Specific Responses to Listerial Infection

### Materials and Methods

Female C3HeB/FeJ mice were obtained from The Jackson Laboratory (Bar Harbour, Me.). For each experiment, mice were age matched and used between 8-16 wks of age. Mice were housed in micro-isolator cages with laboratory chow and water available ad libitum.

Specificities and sources for antibodies were as follows: PE-conjugated anti-CD5 (clone 53.7.3, see ref (53)) and anti-CD3 (clone 145-2C11, see ref (54)); FITC-conjugated anti-CD45R/B220 (clone RA3-6B2, see ref (55)), anti-TCR β chain (clone H57-597, see ref (56)), anti-γδ TCR (clone GL3, see refs. (57,58)), anti-CD4 (clone RM4-5, see reference 59) and anti-CD8 (clone 53-6.7, see reference 60) antibodies, and all isotype control antibodies were obtained commercially (Pharmingen, San Diego, Calif.); anti-IFN-γ hybridomas R4-6A2 (American Type Culture Collection or ATCC, Rockville, Md., ATCC #HB 170) Rockville, Md. (61) and XMG1.2 (62) were provided by DNAX Inc. (Palo Alto, Calif.); anti-mouse IL-12 hybridoma C17.8.20.15 was provided by Dr. G. Trinchieri (Wistar Institute, Philadelphia, Pa.). mAb generated from cell lines in the laboratory were purified from culture supernatants by protein A or protein G affinity chromatography (63). Purified antibodies were directly conjugated to biotin, for use in ELISA assays, using standard techniques (63).

The composition of individual antigen/cytokine mixtures and the immunization schedules employed are described in the Results section. All injection doses were prepared in a total volume of 1 ml, using endotoxin-free PBS, and were administered intraperitoneally. Murine rIL-12 used in these studies was a kind gift from the Genetics Institute (Andover, Mass.).

Experimental Listeria monocytogenes infections were established by i.p. injection of a sub-lethal dose (target dose of 6×10³) of viable L. monocytogenes strain 43251 (ATCC). Mice were challenged by i.p. injection of a large dose of viable L. monocytogenes (target dose of 4-6×10⁵, approximately 10×LD₅₀, for C3HeB/FeJ mice). Listeria used for injection were grown overnight in brain heart infusion (BHI) broth (Difco Laboratories, Detroit, Mich.) at 37° C. with aeration. Bacteria were washed three times in PBS and concentrations were determined by optical density with confirmation by colony counts on BHI agar plates. Heat-killed Listeria monocytogenes (HKLM) were prepared by incubating the bacteria at 80° C. for 1 hour. The heat-killed bacterial preparations were tested for lack of viability on BHI agar plates. Prior to killing, the bacteria were washed three times and resuspended in LPS-free PBS.

Soluble listerial protein (SLP) was prepared as previously described (64). Briefly, L. monocytogenes was grown overnight at 37° C. in BHI broth. Bacteria were pelleted by centrifugation, washed in PBS, and resuspended in a small volume of PBS. The suspension was then sonicated, the particulates were pelleted by centrifugation and discarded, and the supernatant was dialyzed against PBS. The supernatant was then banded on cesium chloride by means of isopycnic gradient centrifugation, and the protein-containing fraction was identified, collected, and dialyzed against PBS.

Peritoneal exudate cells (PEC) were obtained from mice by peritoneal lavage with HBSS supplemented with 0.06% BSA, 10 mM Hepes buffer (Irvine Scientific, Santa Anna, Calif.), 50 U/ml penicillin, 50 μg/ml streptomycin, and 10 U/ml heparin. Cells from all mice within an immunization group were pooled. Macrophages were isolated by incubating PEC (4×10⁶/ml/well) in 100 mm tissue culture-treated plates (Corning Inc., Corning, N.Y.) for 2 hours at 37° C. Non-adherent cells were removed and pooled resulting in cell populations designated as plastic non-adherent peritoneal exudate cells (PNA). Following peritoneal lavage, spleens were removed by sterile dissection.

PNA were suspended (1.5×10⁶/ml) in culture media (RPMI 1640 supplemented with 10% FCS, 5×10⁻⁵M 2-ME, 0.5 mM sodium pyruvate, 10 mM Hepes buffer, 50 U/ml penicillin, 50 μg/ml streptomycin, and 2 mM L-glutamine) and a variety of in vitro stimulants were added at pre-determined optimal doses (as indicated in figure legends) in 24-well plates (Corning Inc., Corning, N.Y.). Reagents used as stimulants in vitro were as follows: Con A (2 μg/ml) purchased from Sigma Chemical Co. (St. Louis, Mo.), HKLM (107/ml), and SLP (8 μg/ml). The cell cultures were incubated at 37° C. for 24 hours, then supernatants were frozen (−20° C.) and saved for use in IL-2 and IFN-γ quantitation assays.

IL-2 was quantitated in PNA culture supernatants using a previously described bioassay (65,66). Briefly, supernatants from PNA cultures were transferred into 96-well tissue culture plates along with 1×10⁴ HT-2 cells (an IL-2-dependent T cell line) in a total volume of 200 μl of culture medium and incubated at 37° C. [^{3H}]Thymidine (1 μCi/well) was added after 24 hours of culture and cells were harvested 6-18 hours later onto glass fiber filters using a Filtermate Cell Harvester (Packard Instrument Co., Inc., Downers Grove, Ill.) and counted using a Matrix 9600 Direct Beta Counter (Packard Instrument Co.). IL-2 concentration in the supernatants was related to a standard curve generated from wells containing varying concentrations of IL-2. The source of the IL-2 standards was supernatant from P815-IL-2 (67) cultures; the IL-2 concentration of this supernatant was measured using the described assay with comparison to known concentrations of human rIL-2 (a gift from Immunex Corp.; Seattle, Wash.). Typically, this assay was linear to approximately 500 U/ml and the lower detection limit was approximately 5 U/ml. All assays were performed in triplicate, and results were reported as the mean (+/−SD) of the triplicate samples.

IFN-γ was measured in PNA culture supernatants using a sandwich ELISA assay, as described by Cherwinski et al. (62). R4-6A2 served as the capture antibody and biotinylated XMG1.2 as the detection antibody. StrepAvidin-peroxidase (Sigma Chemical Co.) was used to amplify the biotin signal and Developer Buffer (600 μg ABTS and 0.02% hydrogen peroxide in 50 mM citrate buffer) was used to develop the assay. Absorbance at 405 μm was read using a SpectraMax 340 automated plate reader (Molecular Devices, Sunnyvale, Calif.). IFN-γ concentration in the supernatants was related to a standard curve generated from wells in which varying concentrations of murine rIFN-γ (Genzyme Corp., Cambridge, Mass.) was captured. Typically, the assay was linear to approximately 120 U/ml and the lower detection limit was 8 U/ml. All assays were performed in triplicate, and results were reported as the mean (+/−SD) of the triplicate samples.

Flow cytometry was performed as previously described (68,69). Briefly, PNA (1-5×10⁵) were incubated on ice for 30 minutes with 25 μl of pre-determined optimal concentrations of fluorochrome-conjugated mAb, washed twice with PBS containing 3% FCS and 0.1% sodium azide, and fixed with 1% paraformaldehyde in PBS. Samples were analyzed on a FACScan® (Becton Dickinson & Co., Mountain View, Calif.) using forward scatter/side scatter gating to select the lymphocyte population for analysis. Isotype-matched control Ig was used to determine background immunofluorescence levels for each test antibody.

### Results

The murine model of listeriosis has been well established and is among the most popular systems available for studying the mechanisms of cell-mediated immune responses. It has been well documented that mice infected with a sub-lethal dose of viable Listeria rapidly clear the infection and are left with long-lived protective listerial immunity (70-72). In contrast, inoculation of mice with high doses of viable Listeria results in systemic infection that is characterized by uncontrolled replication of bacteria in the spleen and liver for 2-4 days, culminating in death between days 4-10 post-infection (71,73). Because the number of bacteria in the spleen and liver is inversely related to the immune status of the mouse, enumeration of Listeria in these organs 2-4 days post infection is an accepted method for determining the susceptibility of mice to listerial infection (74). Specific acquired immunity to Listeria is characterized by a 2-4 log₁₀ reduction in bacterial load in the spleen or liver, in comparison to the bacterial load observed in susceptible mice. It is also important to note that although HKLM (and other listerial subunit preparations) are known to be poorly immunogenic when administered alone (75,76), co-administration of such antigens with an effective adjuvant can result in production of protective immune responses (68,69,77). Therefore, in addition to its utility as a model for study of cell-mediated immunity, the murine model of listeriosis is also an excellent model for evaluating the efficacy of vaccine formulations or immunotherapy strategies.

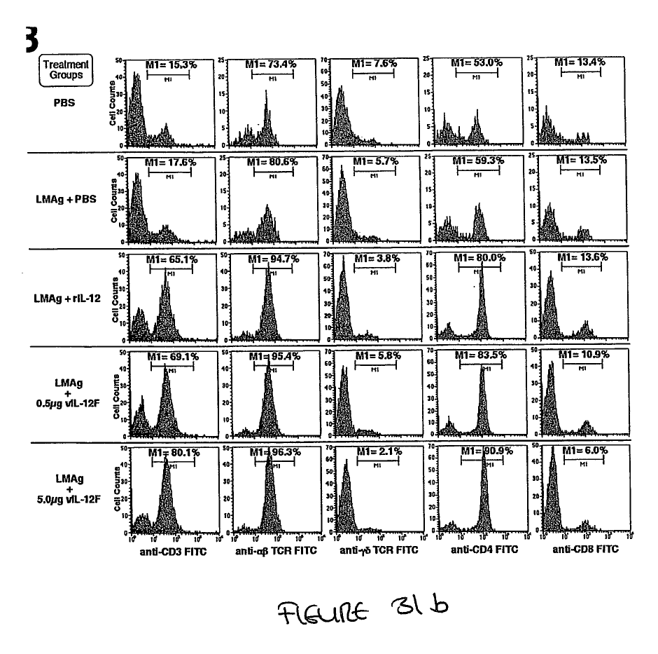

To evaluate the adjuvanticity of vIL-12F, we immunized C3HeB/FeJ mice (5/group) with either PBS, 10⁹ HKLM/10 μg SLP (LMAg)+PBS, LMAg+0.5 μg rIL-12, LMAg+0.5 μg vIL-12F, or LMAg+5.0 μg vIL-12F on days 0, 5, and 15. On day 20, mice were killed and tissues were collected for analysis. PECs were collected by lavage and pooled, PNA populations were prepared, and PNA were restimulated in vitro with a series of stimuli (culture media: no further stimulation; Con A: a polyclonal T cell stimulator; and HKLM or SLP: listerial antigen preparations) at predetermined optimal doses for 24 hours at 37° C. As a measure of immune responsiveness, cell-free supernatants from these cultures were analyzed to quantitate IL-2 (FIG. 30A) and IFN-γ (FIG. 30B) produced by T cells in response to the indicated in vitro stimuli.

As observed in previous studies (68,69,77), the mice immunized with LMAg+rIL-12 (positive control) produced listerial antigen-specific T cells similar to those produced by mice that have been infected with a sub-lethal “immunizing” dose of viable Listeria. Also as expected, the mice immunized with PBS alone or LMAg+PBS (negative controls) failed to mount any detectable Listeria-specific T cell responses. The pattern of reactivity of T cells from mice that received vaccine formulations of LMAg+either 0.5 μg vIL-12F or 5.0 μg vIL-12F was very similar to the pattern produced by T cells from the LMAg+rIL-12 (positive control) group. In addition, the responses produced by these two test groups appeared to be somewhat vIL-12F dose-dependent; T cells from mice that received LMAg in combination with the higher dose of vIL-12F produced larger quantities of IL-2 and IFN-γ following stimulation in vitro with listerial antigens. These findings correlated with the general state of immune stimulation that was suggested by the relative spleen sizes of mice from each immunization group. Compared to the two negative control treatment groups (PBS, LMAg+PBS), dramatic increases in spleen size were observed in each of the positive control mice (LMAg+rIL-12) as well as each of the mice immunized with LMAg and vIL-12F (data not shown). Moreover, a vIL-12F-dependent dose response was evident as the splenomegaly observed in mice that received LMAg+5.0 μg vIL-12F was even more pronounced than in the mice that received the lower dosage of vIL-12F (LMAg+0.5 μg IL-12F). These results indicated that vIL-12F is similar to native rIL-12 in its ability to promote immune responsiveness to a non-immunogenic antigen mixture.

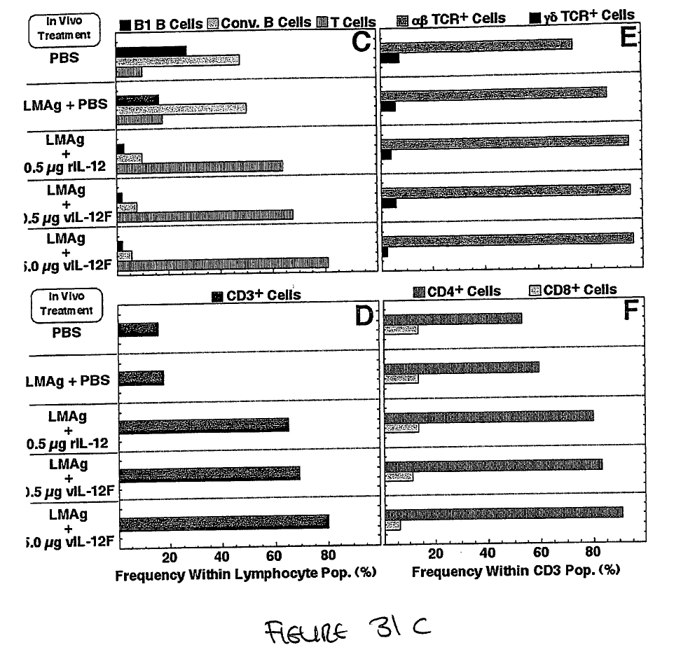

As an additional measure of the immune status of mice immunized with LMAg and vIL-12F, flow cytometric analysis was performed to characterize the cell populations resident in the peritoneal cavity (PNA) of each immunized animal. Analysis of PNA double-stained with anti-CD5 and anti-CD45R/B220 revealed a marked increase (3-4 fold) in the frequency of CD5+ cells (T cells) with a concomitant decrease in CD45R/B220+ (B cells) and CD5lo/B220lo (B1 B cells) in mice immunized with LMAg and either rIL-12 or vIL-12F (compared to the frequencies observed in mice from negative control immunization groups; FIGS. 31A and 31C). To further characterize the CD3+ peritoneal cell populations of these animals, PNA were double stained with anti-CD3 and either anti-αβ TCR, anti-γδ TCR, anti-CD4, or anti-CD8 and flow cytometric analysis was performed (FIG. 31B). As observed for the CD5 staining patterns, the CD3 staining patterns revealed that the frequency of CD3+ cells within the peritoneal lymphocyte population was dramatically higher (3-4 fold) in animals that received LMAg and either rIL-12 or vIL-12F, than in mice that received a negative control formulation (FIGS. 31B and 31D). In addition, mice that received LMAg and either rIL-12 or vIL-12F experienced a selective increase in peritoneal frequency of αβ TCR+/CD4+ cells (FIGS. 31E and 31F, respectively). The results reported here revealed that the alterations in peritoneal cell frequencies elicited by co-administration of LMAg with vIL-12F closely mimicked the alterations elicited by LMAg+rIL-12 treatment. Moreover, the alterations in peritoneal cell frequencies experienced by the mice that received LMAg+rIL-12 (positive control) in the current report are consistent with those observed and reported previously (68,69,77).

### Example 11

### Administration of LMAg+vIL-12F Confers Long-Lived Protective Listerial Immunity

### Materials and Methods

Following sterile removal from immunization/challenge recipients, each spleen/liver was homogenized using a ground glass homogenizer. Cells were disrupted by treatment with 0.5% Triton X-100 (Sigma Chemical Co., St. Louis, Mo.) in a total of 10 ml PBS to release the intracellular bacteria. Serial 10-fold dilutions of each sample were made and 100 μl of each dilution was spread evenly onto BHI plates to quantitate the live Listeria in these organs.

### Results

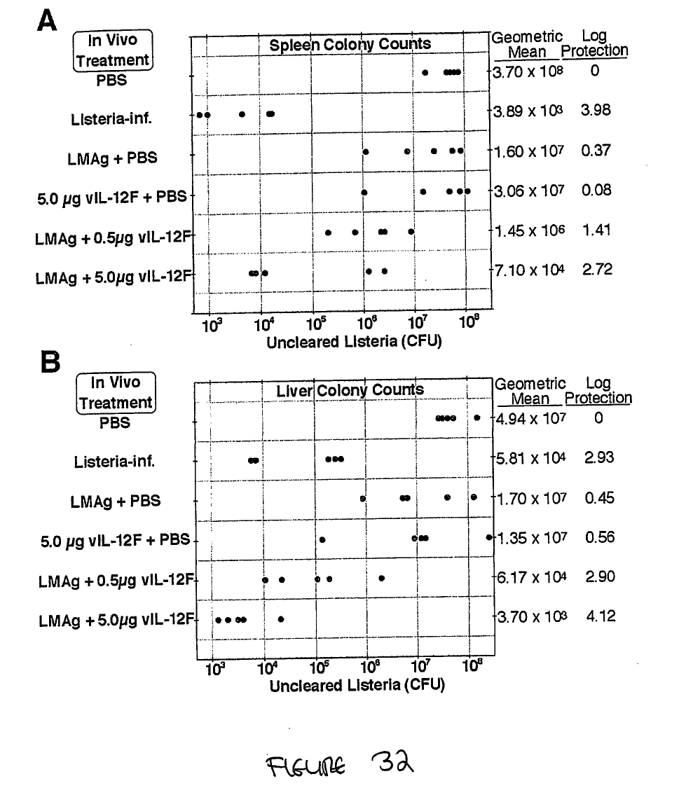

To determine whether the co-administration of vIL-12F with a typically non-immunogenic antigen could confer a protective immune response, C3HeB/FeJ mice (5/group) were immunized with either PBS alone, LMAg+PBS, 5.0 μg vIL-12F+PBS, LMAg+0.5 μg vIL-12F, or LMAg+5.0 μg vIL-12F on days 0, 5, and 15. An additional group of mice received a single sublethal dose (6×10³/mouse or 0.12×LD₅₀) of viable Listeria (Listeria-infected; positive control) on day 0; this group of mice was used a benchmark for the typical acquired immunity that results following recovery from listeriosis. On day 45, the mice were challenged (i.p.) with a lethal dose of viable Listeria (6.4×10⁵ or 12.9×LD₅₀). The mice were killed on day 49, and the bacterial load in the spleen (FIG. 32A) and liver (FIG. 32B) of each mouse was determined. The bacterial loads observed in the organs of the mice that received LMAg+5.0 μg vIL-12F were dramatically reduced compared to the PBS control group (2.72 and 4.12 log₁₀ reduction in the spleen and liver, respectively). In fact, these results were similar to those observed in animals that received an immunizing dose of viable Listeria (positive control group) in which animals experienced a 3.98 and 2.93 log₁₀ reduction of bacterial load in their spleens and livers, respectively. These results indicated that mice immunized with LMAg+5.0 μg vIL-12F produced a protective Listeria-specific immune response. Immunization with LMAg+0.5 μg vIL-12F appeared to promote partially protective immune responses as evidenced by a moderate reduction of bacterial load in the spleen and liver of each animal (1.41 and 2.90 log₁₀ reduction, respectively). As expected, little to no reduction in bacterial load was ₁₀observed in the spleens and livers of mice immunized with LMAg+PBS (0.37 and 0.45 log reduction, respectively) or vIL-12F+PBS (0.08 and 0.56 log₁₀ reduction, respectively). The protective immunity (and the vIL-12F-dependent dose response) observed in this experiment correlated well with the presence of Listeria-specific T cells (as indicated by IL-2 and IFN-7 production by restimulated peritoneal lymphocytes in vitro; FIG. 31) in mice immunized with LMAg+vIL-12F.

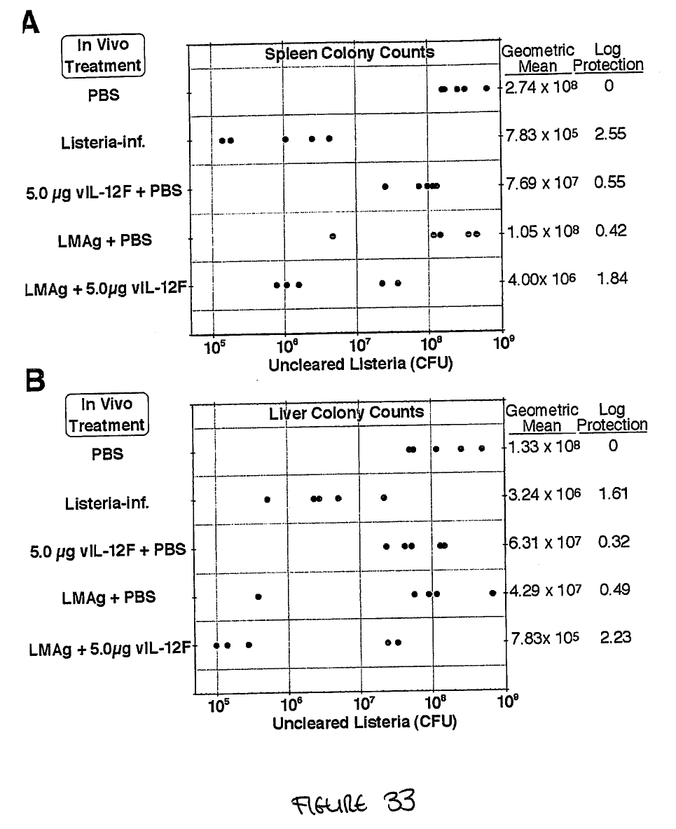

To determine whether the protective immunity conferred by immunization with LMAg+vIL-12F was long-lived, C3Heb/FeJ mice (5/group) were immunized with LMAg+5.0 μg vIL-12F or appropriate control formulations on days 0, 5, and 15. An additional group of mice received a single immunizing dose (6×10³/mouse or 0.12×LD₅₀) of viable L. monocytogenes (Listeria-infected; positive control) on day 0. More than three months after the final booster immunization was administered (day 120), each mouse received (i.p.) a large challenge dose (3.8×10⁵ or 7.6×LD₅₀) of viable Listeria. Four days later (day 124), the mice were killed and the bacterial load in the spleen and liver of each mouse was quantified as a measure of susceptibility to L. monocytogenes. Similar to the results observed in the short-term protective immunity trial (FIG. 33) described above, a dramatic reduction of bacterial load (compared to the PBS treatment group) was observed in the spleens and livers of mice immunized with either LMA-g+vIL-12F (1.84 and 2.23 log10 reduction, respectively) or an immunizing dose of viable Listeria (2.55 and 1.61 log 10 reduction, respectively), but not those immunized with either LMAg or vIL-12F alone (FIG. 33). These results demonstrate that immunization with LMAg+vIL-12F confers long-lived protective immunity similar to that elicited by sub-lethal infection with viable Listeria.

### Example 12

### rVSV-DsRed Exhibits Strong Cytotoxic Activity In Vitro Against C6 Gliomas

### Materials and Methods

The C6 glioma cell line (American Type Culture Collection, Manassas, Va.) was maintained as a monolayer culture at 37° C., 5% CO₂ in Hams/F12 supplemented with 15% heat-inactivated horse serum, 2.5% heat-inactivated fetal bovine serum, 100 i.u./ml penicillin, and 100 μg/ml streptomycin. The C6 glioma cell line was stably transduced with the pFB retrovirus (Stratagene, La Jolla, Calif.) expressing green fluorescent protein (GFP) to allow for enhanced visual analysis. Cells stably transduced with GFP were sorted using flow cytometry to generate a cell population homogeneously expressing high levels of GFP. To prepare the cells for seeding onto the slice culture, cells in exponential growth were harvested by EDTA/Trypsin for 5 minutes at 37° C. Trypsinization was terminated with the complete media described above and the cells were centrifuged for 5 minutes at 1,000 RPM. The pellets were resuspended in sterile phosphate buffered saline (PBS) and counted using Trypan blue staining methods. The pellets were then resuspended in PBS at a concentration of 3×10⁴ cells/μl and placed on ice until seeded onto the slice culture.

To follow virus infection in real-time we constructed an rVSV that expresses the cDNA for the red fluorescent protein, DsRed, which is a commercially available chromoprotein derived from a colored sea anemone-Discosoma sp. (BD Biosciences-Clontech). The cDNA for the DsRed protein was excised from the parent plasmid and subcloned into the multiple cloning site of pVSV-MCS 2.6, which is the parent vector to pVSV-ΔG-GSHA/GFP which has been described previously (3). The resulting recombinant virus, rVSV-DsRed was recovered and characterized using standard protocols established previously in our laboratory and described elsewhere (78). To construct rVSV-ΔG-DsRed we subcloned the cDNA for DsRed into the multiple cloning site of pVSV-ΔG-PL 31 located upstream of L gene. The recombinant virus rVSV-ΔG-DsRed was then recovered using methodologies described previously (3). Because ΔG-DsRed does not encode the viral glycoprotein, virus is propagated in cells transiently expressing VSV G protein. Therefore, infection of cells utilizes VSV G protein-complemented virus.

C6 Glioma cells were plated in triplicate in 96-well flat-bottom plates at 30%, 60%, and 90% confluency in a 100 μl total volume of Hams/F12 medium supplemented with 100 U/ml of penicillin and 100 μg streptomycin, 15% horse serum, and 2.5% fetal bovine serum. Cells were incubated overnight at 37° C. to allow for adherence. The cultures were inoculated with varying amounts (10¹ to 10⁵ pfu) of rVSV-DsRed. Following addition of the virus inoculum, cell death was analyzed at 4, 8, 24, 36, 48, 72, and 96 h post-infection using the CellTiter 96R Non-Radioactive Cell Proliferation assay (G5421, Promega, Madison, Wis.). In this assay, the compound MTS [(4,5-dimethylthiazol-2-yl)-5-(3-carboxymethoxyphenyl)-2-(4-sulfonphenyl) 2H Tetrozolium] is mixed with the electron coupling reagent phenazine methosulfate in a 20:1 ratio and added to the 96 well plate culture. The MTS reagent is converted by living cells into an aqueous soluble formazan by dehyrdrogenase enzymes in metabolically active cells. Thus the number of living cells is directly proportional to the amount of formazan produced which is read at 490 nm. The percentage of viable cells present in the culture at each time point was calculated by comparing the absorbance value at 490 nm from the MTS assay for each condition with untreated control cells using a Lab Systems Multiskan Biochromatic Elisa plate reader (Vienna, Va.). All described values represent the average of three data points.

### Results

To confirm that C6-GFP glioma cells were susceptible to infection by rVSV-wt encoding DsRed, we infected cells grown in a 6-well dish with amounts of virus ranging from 10¹ to 10⁵ pfu. FIG. 33 shows the time course of infection of C6 gliomas at 10⁵ pfu. Most cells showed the characteristic cytopathic effects of VSV infection, i.e. cell rounding and cell death by 24 hours post-infection. Greater than 95% cells lifted off the dish by 48 hours. Infection with a lower viral inoculum produced similar cytopathic effects but with slower kinetics (data not shown). These results indicate that C6 glioma cells are sensitive to infection by rVSV-DsRed.

To better quantify the degree of cytoxicity of VSV for C6 glioma and more specifically define the time course, we used in vitro cytotoxicity assays. C6-GFP glioma cells were plated on 96-well flat bottom plates at 30%, 60%, and 90% confluency and were infected with varying amounts of rVSV-DsRed ranging from 10 to 10⁵ pfu. Cell death was analyzed at 4, 8, 24, 36, 48, 72, and 96 h post-inoculation using the Cell Titer Non-Radioactive Cell Proliferation assay. As shown in FIG. 33, rVSV-DsRed resulted in roughly 90% cell death within 72 hours irrespective of viral titer. The results were similar when cells were plated at 30% and 60% confluencies (data not shown). In summary, rVSV-DsRed showed excellent in vitro cytolytic activity against rat C6-GFP gliomas irrespective of cell density and the amount of virus inocula.

### Example 13

### An Organotypic Slice-C6-GFP Coculture System for Studying Efficacy and Toxicity of VSV-Based Anti-Glioma Therapies

### Materials and Methods

Organotypic brain slice culture methods were modified from those introduced by Plenz and Kitai (79). 1-2 day old Sprague-Dawley rat pups were decapitated, brains were removed rapidly, and kept in Gey's balanced salt solution (G9779, Sigma-Aldrich Corp., St. Louis, Mo.) with 0.5% dextrose at 4° C. Coronal slices were made on a vibratome at 500 μm for striatum and substantia nigra and 400 μm for cortex in Gey's/dextrose solution. The slices with areas of interest were cut under a dissecting microscope. The areas of cortex, striatum, and substantia nigra pars compacta were dissected into 0.5-1 mm size and were subsequently placed on Millicell culture insert (PICM03050, Millipore Corp., Billerica, Mass.) and submerged in 10 μl of chicken plasma (P3266, Sigma-Aldrich Corp., St. Louis, Mo.) on a cover-slip. After carefully aligning the tissue on the insert, 0.5 unit of bovine thrombin (T6634, Sigma-Aldrich Corp., St. Louis, Mo.) in 10 μl of Gey's/dextrose solution was added and mixed with the chicken plasma on the coverslip. The coverslip with the tissue was then placed into a culture tube (156758, Nalge Nunc International, Rochester, N.Y.), and to each tube was added 750 μl of incubation medium, which has the following components (all from Invitrogen Corp., Carlsbad, Calif.): 50% basal medium Eagle (BME) (21010-046), 25% Hanks' balanced salt solution (HBSS) (24020-125), 25% horse serum (26050-070), 1 mM L-glutamine (25030-081), and 0.5% dextrose (15023-021). The tubes were then incubated at 35° C. on a carousel rotated at a speed of 0.5 RPM. After 72 hours of culture, a mitosis inhibitor mix comprised by 4.4 μM each of cytosine-α-D-arabinofuranoside, uridine, and 5-fluro-2′-deoxyuridine was added into the culture medium. This was removed after 24 hours and replaced with 750 μl of fresh medium. The culture media was completely replaced twice a week thereafter. The slices were used for experiments after three weeks in culture. To generate a slice-C6-GFP glioma coculture, 150,000 GFP-positive C6 rat glioma cells in a volume of 5 μl were inoculated onto the slice under sterile conditions. The culture tube was placed horizontally without medium in the incubator for 30 minutes. The culture medium was then replaced and the tube was left in a horizontal position for another 2 hours before resuming revolution on the carousel. The growth of GFP positive C6 glioma cells in culture was visualized using an Olympus fluorescence microscope and the images were collected using a digital camera.

The brain slice culture, with or without C6-GFP glioma cells, which had grown for at least three weeks in culture, were infected with 10⁴ plaque-forming units (pfu) of rVSV-DsRed or 10⁶ infectious units (IU) of Infectious ΔG-DsRed viruses. The viruses were adsorbed on the slices for 5 hours at 37° C. on the carousel. The inoculum was then removed, the slices were rinsed in media and fresh media was added to the slices. The slices were then incubated for 3 additional days. For slices receiving IFN-β, the slices were pretreated with 1,000 U of rat IFN-β at 18-20 hours prior to virus infection. The virus was then adsorbed in presence of IFN-β as described above. The inoculum was then replaced after 5 hours with fresh media also containing 1,000 U of IFN-β.

Slices were fixed in 4% paraformaldehyde in phosphate buffer for 2 hours and washed with phosphate buffered saline (PBS) for 3 times before the tissue was mounted onto a glass slide. The cultured tissue was dried on a hot plate briefly and stored at 4° C. for later use. The immunohistochemistry was performed with the following procedures. The slides were briefly rinsed in PBS; treated with 3% hydrogen peroxidase and 10% methanol for 20 minutes with 3 subsequent rinses with PBS; incubated in 2% non-fat milk and 0.3% Triton-X in PBS for 1 hour; incubated in mouse anti-microtubule-associated protein 2 (MAP-2, 1:500, M-4403, Sigma-Aldrich Corp., St. Louis, Mo.) or mouse anti-tyrosine hydroxylase (TH, 1:1,000, MAB318, Chemicon International, Temecula, Calif.) with 3% donkey serum and 0.1% Triton-X overnight at room temperature; washed with PBS for 3 times; incubated in Cy™2 or Cy™3-conjugated Affinipure donkey anti-mouse IgG (1:250, Jackson ImmunoResearch Laboratories, Inc., West Grove, Pa.) with 2% donkey serum and 0.1% Triton-X for 4 hours at room temperature in the dark; washed with PBS for 3 times; and dehydrated through graded ethanol, cleared with xylene, and mounted with a coverslip in DPX mounting medium (44581, Fluka Biochemika). The MAP-2 and TH immunoreactivity was visualized with a Bio-Rad confocal microscope and digital images were collected using the associated confocal software. The extent of virus infection in the slices was visualized by following DsRed expression using a fluorescence microscope.

### Results

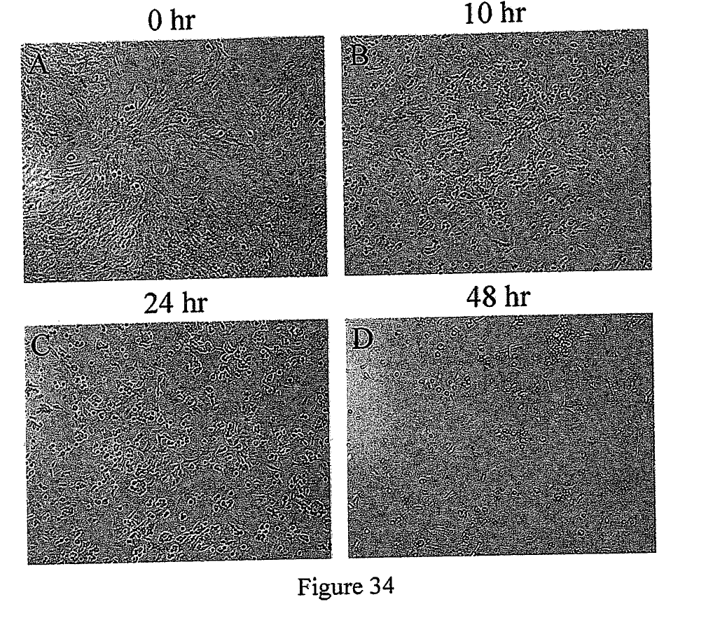

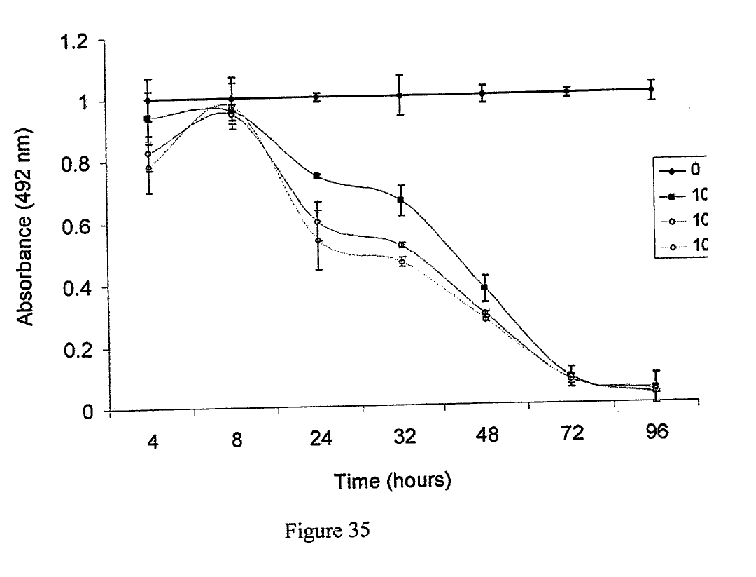

An organotypic brain slice-glioma coculture (FIG. 34), established for 3 weeks, is viable for up to 6-8 weeks post-plating, with demonstrable TH immunostaining indicative of mature substantia nigra neurons in the slice (FIG. 34B). FIG. 35C demonstrates baseline MAP-2 immunoreactivity in this organotypic slice culture model, and serves as a non-specific neuronal marker that is a sensitive indicator of neuronal integrity (80). Glioma cells stably expressing GFP were grown naturally over and through the slice tissues, and followed in real-time with fluorescence microscopy (FIGS. 34D and E) where, after a period of 3 to 4 days, depending on the C6 glioma inoculum size and rate of tumor cell growth, various facets of slice-glioma cell biology can be studied, including the effect of therapeutic agents on tumor regression and slice integrity.

Wild-type VSV is toxic to normal tissue in the slice culture system. rVSV-DsRed expresses a red fluorescent protein, thus providing a distinguishable signal from the GFP (green) fluorescence from the C6 glioma cells, which was followed during viral infection of the slice culture in real-time. The gene for DsRed was introduced between the G and L genes in the VSV genome. Insertion of an additional foreign gene between the G and L genes has no effect on the cytolytic properties or on the replication efficiency of the virus (81). Following infection, the slice cultures were examined by immunohistochemical staining for MAP-2 expression. rVSV-DsRed readily infected the slice and by the third day most of the cells in the slice were infected as indicated by the red fluorescence seen in the tissues (FIG. 35A). Not surprisingly, the MAP-2 immunostaining for the infected slice was poor, indicating loss of neuronal tissue integrity (FIG. 36A). Thus replication competent, wild-type VSV is quite cytopathic to neuronal tissues and therefore its application as an oncolytic agent would necessitate the use of an antiviral agent to protect the normal tissues from its toxicity.

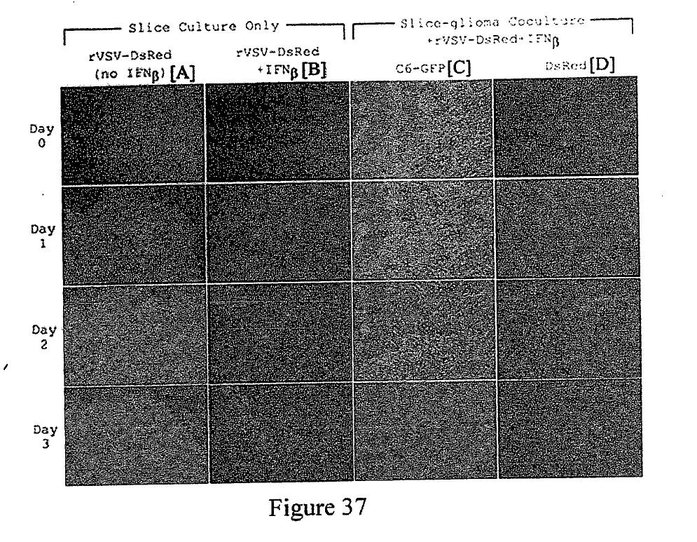

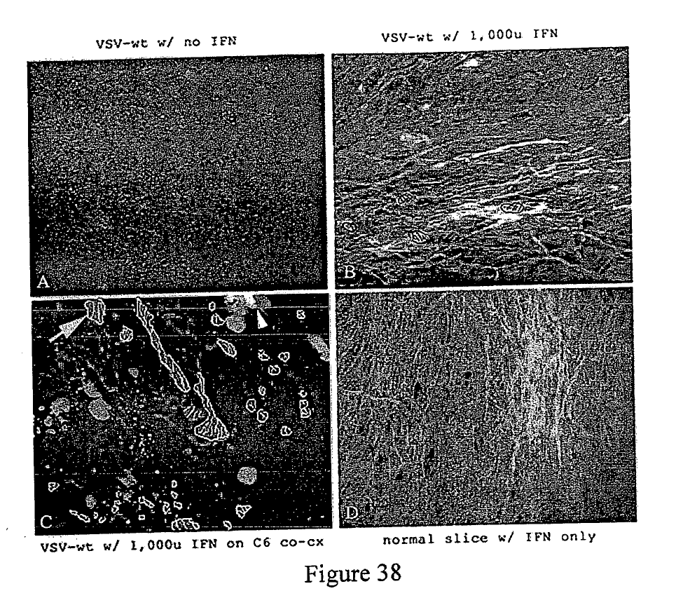

It is known that VSV is highly sensitive to the antiviral effects of IFN-β. Malignant cells of various lineages have one or more defects in the IFN signaling pathway (82,83), which has recently been exploited for specific tumor cell targeting by VSV-mediated cytolytic activity, while normal tissue is unaffected (84,85). The organotypic slice coculture system, was therefore utilized to determine whether IFN-β protected normal neuronal tissue from VSV infection yet was toxic to glioma cells in the culture. Slice cultures were pretreated with 2 different concentrations of IFN-β (100 U or 1,000 U) for 24 hours prior to VSV infection. Cultures pretreated with 1,000 U of IFN-β were protected from infection with rVSV-DsRed (FIG. 36). The MAP-2 staining pattern was also improved dramatically following IFN-β treatment prior to infection, indicating a significant decrease in VSV-mediated cytotoxicity to normal tissues (FIG. 37B) and consistent with the anti-apoptotic function of IFN-β (86). Pretreatment with 100 U of IFN-β was beneficial, though higher doses were more protective (data not shown). IFN-β alone was not toxic to the slice, providing an 15 attractive pre-treatment strategy in conjunction with replication-competent wild-type VSV (FIG. 37D).

Glioma cells grown on mature slice cultures for 4 developed into a sizeable tumor mass. Pretreatment with 1,000 U of IFN-β for 24 hours followed by rVSV-DsRed infection resulted in a reduction of C6-GFP glioma tumor load without infection of normal tissues by rVSV-DsRed (FIG. 36). Thus, rVSV-DsRed appeared to selectively destroy the C6-GFP tumor. Interestingly, despite little apparent infection of the slice with VSV in IFN-β pretreated cocultures, the slices were still signifcantly damaged as indicated by the aberrant MAP-2 staining pattern (FIG. 37C). This indicated that while rVSV-DsRed was effective at eliminating tumor cells, there was still sufficient damage to the slice tissues mediated by mechanisms other than those typically associated with viral infection.

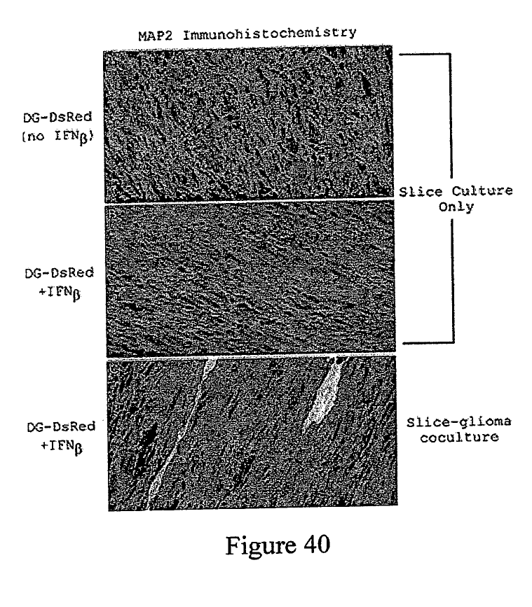

Replication-restricted VSV-ΔG is similar to wild-type VSV in tumor cytolytic activity, but is superior with respect to toxicity in the organotypic slice-glioma coculture system. rVSV-ΔG is a second-generation, replication-restricted VSV. rVSV-ΔG lacks the glycoprotein (G) gene which encodes for the envelope protein of the virus. The glycoprotein (G protein) of VSV mediates attachment of the virus to cells and fusion of the viral envelope with the endosomal membrane following endocytosis of the virus and, as such, is required for VSV infectivity. Therefore, to propagate rVSV-ΔG vectors, the virus must be grown in cells transiently expressing the wild-type G protein. The progeny viruses that are produced contain the transiently expressed G protein in the viral envelope (infectious ΔG virus) and can infect cells normally; however, since the genome of these viruses lack the G protein coding region, the progeny virions that are released from cells that do not express G protein are non-infectious and cannot re-infect adjacent cells. Therefore, rVSV-ΔG vectors undergo a single round of replication (e.g. replication-restricted). The advantage of using rVSV-ΔG is that the exponential increase in virus particles generated over time with replication competent virus, such as rVSV-DsRed is avoided. To test the toxicity of replication-restricted VSV-ΔG in the slice culture system we inoculated the culture with a modified ΔG virus encoding DsRed (ΔG-DsRed) and examined the slice culture after 3 days. The culture was fixed and stained for MAP-2 to determine neuron integrity following infection. We found that infectious ΔG-DsRed readily infected the slice by the second day in a manner similar to that seen with rVSV-DsRed (FIG. 39A). Interestingly, despite the significant infection resulting from ΔG-DsRed, MAP-2 immunoreactivity remained relatively intact (FIG. 40A). As predicted, little to no infection by ΔG-DsRed was observed when the slices were pretreated with IFN-β (FIG. 39B) and MAP-2 staining was similar to that seen in uninfected cultures (FIG. 40B).

C6-GFP glioma cells grown on mature slice for 4 days produced a sizeable tumor mass, however pre-treatment with IFN-β at a concentration of 1,000 U for 24 hours followed by inoculation of 10⁶ IU of infectious ΔG-DsRed virus demonstrated a significant reduction in tumor load 3 days post incubation with ΔG-DsRed virus (FIG. 39) Despite excellent cytolytic activity against the tumor, little if any infection of normal cells in the slice culture itself occurred (FIG. 39D). Thus, IFN-β pretreatment followed by infection with infectious ΔG-DsRed selectively destroyed the C6-GFP glioma without infecting normal tissue, which was further corroborated by MAP-2 immunoreativity results (FIG. 39C). Thus, the use of a replication-restricted ΔG-VSV, in combination with IFN-β pretreatment, is an attractive combination therapy for treatment of glioma.

### Example 14

### Efficacy of VSV-Based Anti-Glioma Therapies

### Materials and Methods

Cells and virus are injected into the appropriate anatomical intracranial area using the implantable guide-screw system (87). Adult female Sprague-Dawley rats 5-7 weeks old, 250-350 grams in weight are used. Animals are anesthetized intraperitoneally with a ketamine/xylazine solution (200 mg ketamine, 20 mg xylazine in 17 ml saline) at a dosage of 0.15 mg/10 g body weight. The cranial area of each rat are shaved and cleaned with povidine-iodine. Animals are immobilized on a stereotaxic frame. A midline incision approximately 5 mm in length is made, with extracranial tissues mobilized to locate the sagital and bregma sutures. A small burr hole is created with a drill 3 mm lateral from midline along the bregma suture. A small canula (Plastics One) is inserted into the burr hole. Phosphate buffered saline (PBS) alone or 1×10⁵ GFP positive C6 glioma cells resuspended in PBS in a volume of 10 μl is injected over a period of 5 minutes at a depth of 5 mm using a Hamilton syringe. Once in place, the canula stopper is inserted to plug the hole. Animals are observed daily for signs of infection or neurologic deficit as a result of tumor growth.

After a 10-day interval, rats are treated with a dose of sterile PBS, or with rVSV-RFP. The time point for administration of rVSV-RFP is chosen based on the predicted size of the intracranial tumor. This should represent a time point when the tumor is detectable by neuro-imaging but before significant neurologic deficit has occurred. Rats are anestehtized prior to intracranial viral administration. The previous incision is reopened and extracranial tissues are mobilized, in order to locate the canula. The stopper is removed and PBS or VSV is administered in a volume of 10 μl. This places the virus directly into the bed of the tumor. In vivo IFN-β pretreatment is also assessed, with respect to both intrinsic toxicity and blunting of VSV toxicity.

### Results

GFP positive C6 glioma cells are delivered to the frontal lobes of rats through a previously implanted canula. After a defined period of incubation, the canula is then used to deliver virus to the tumor bed. Animals are sacrificed at selected time points after treatment for analysis. Histopathological studies determine the degree of cytoreduction and CNS toxicity caused by virus. Additionally, parameters such as neurological deficit and survival are used as criteria for defining the outcome. Thus the ability of VSV to destroy cancerous cells in a live CNS background without significant damage to normal tissues is demonstrated.

It is possible that a one-time dose is sufficient to treat the tumor, owing to the use of a replication-competent virus, which would produce an exponential increase in viral load as tumor cells become infected and release progeny virus. This cycle may continue until most of the tumor is destroyed, at which time the immune system would clear remaining virus. The canula system allows for multiple dosing over a period of time, however, hence redosing is another possible course of action for eradication of the tumor.

It is possible that the rat intracranial glioma model provides variable results since a reported 80% success rate, with a range for 60 to 100% among different groups occurs. Use of immunodeficient animals (i.e., nude rats), may increase the success rate for “tumor take” to close to 100%, and may be undertaken, bearing in mind, however, that the immune system is an important component to these studies, and thus are not alone an appropriate model of study. Use of large sample sizes with the intracranial glioma model using immunocompetent Sprague-Dawley rats, should allow for correct interpretation of results.

Animals are assigned to groups according to Table 4.

TABLE 4

| | # | # Cells | Rx at Day | Incubation |
|---|---|---|---|---|
| Group | Animals | InjectedH | 10 = H | Time [symbol] |
| Control Toxicity | 10 | None | Sterile PBS | X days =* |
| Group | | | | |
| VSV Toxicity | 10 | None | VSV at X dose* | X days =* |
| Group | | | | |
| IFN-β Toxicity | 10 | None | Sterile PBS | X days |
| GroupΦ | | | | |
| Control Rx Group | 20 | 1 × 10⁵ | Sterile PBS | 17 days = |
| Treatment Group + | 20 | 1 × 10⁵ | Sterile PBS | 17 days = |
| IFN-β aloneΦ | | | | |
| Treatment Group + | 20 | 1 × 10⁵ | VSV at X dose* | 17 days = |
| VSV alone | | | | |
| Treatment Group + | 20 | 1 × 10⁵ | VSV at X dose* | 17 days = |
| VSV + IFN − βΦ | | | | |

*Dose or time point will be determined,
= Refers to time from initial inoculation of tumor cells, [symbol]
Refers to the time point at which analyses will take place, HTumor cells, Control Rx, and VSV Rx will administered intracranially in a volume of 10 μL.
ΦVSV protection using IFN α/β pretreatment (i.e., dose and timepoint of administration) is assessed, with the dose and timepoint as a function of the results obtained.
X denotes the no. of days for treatment of the control group and will depend on the symptoms of the disease displayed by the animals.

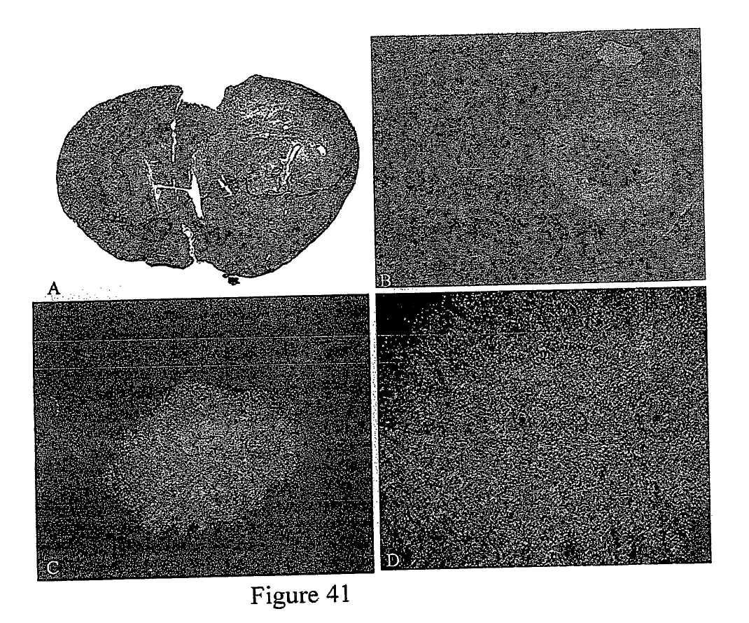

VSV effects following direct injection into the brain are compared to the PBS-only control. Animals are monitored for signs of encephalitis (lethargy, poor feeding, weight loss, etc.) and sacrificed after a specified period of time as determined from results of a pilot study. The tissues will be prepared according to standard protocols. Rats are perfused transcardially under deep anesthesia with heparinized saline, followed by 4% paraformaldehyde. The brain is removed and post-fixed in 4% paraformaldehyde for 2 hours at 4° C. The brain is sectioned on a cryostat, 20 μm sections. Different techniques will be used to examine the brain sections for signs of toxicity including direct fluorescence microcopy for VSV infection (monitoring of RFP fluorescence or immunohistochemistry staining using N protein-specific antibody), hematoxylin & eosin staining for basic neuropathology, and apoptosis studies for cell death in the tumor and surrounding parenchyma. An example of data obtained from control rats containing a significant tumor burden is shown in FIG. 41.

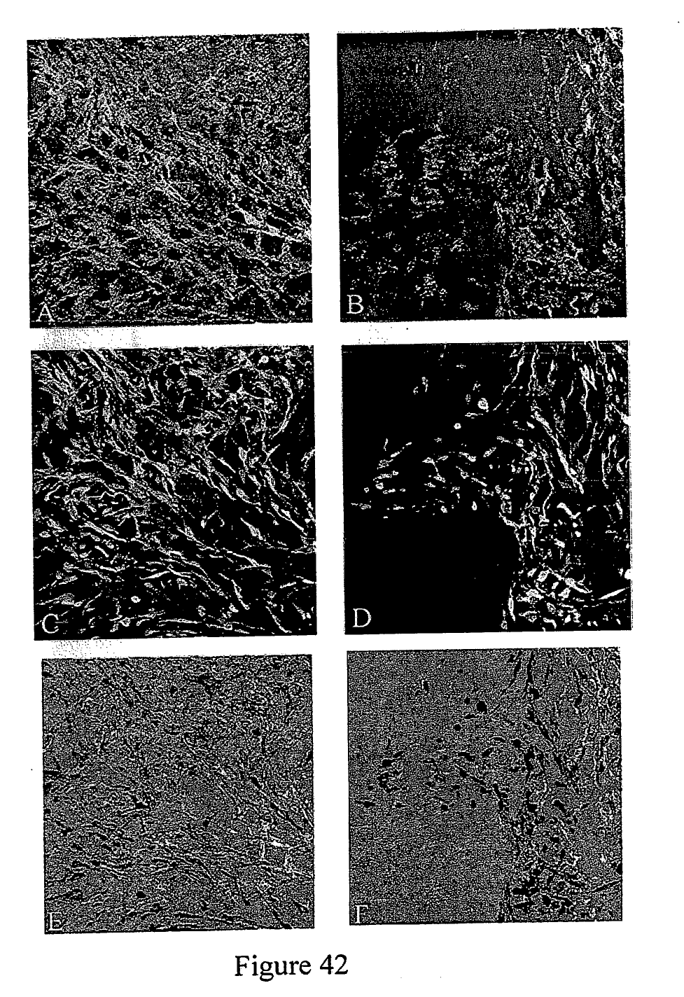

Following a 17-day period of incubation from the initial tumor inoculation (7 days after administration of virus), animals within each study group are sacrificed and analyzed for the histopathological characteristics of the tumor and surrounding tissues. Important parameters such as the size of the tumor, presence or absence of a midline shift, and presence or absence of necrosis in the tumor bed are analyzed. Additionally, the surrounding normal tissues is closely scrutinized for signs of toxicity related to the virus. Examination of brain sections for evidence of tumor growth, including direct fluorescent microcopy for GFP, hematoxylin & eosin staining for basic neuropathology, immunocytochemistry against specific tumor antigens, and apoptosis studies for cell death in the tumor and surrounding parenchyma, are conducted. GFP labelling in C6 glioma cells allows for very sensitive detection and analyses of the tumor mass and for micro-populations of cells that have moved away from the initial site of inoculation (FIG. 42).

An important aspect of this study is the ability of VSV to access and target micropopulations of cells that have escaped the primary site of tumor (FIG. 42). The GFP label carried by the C6 glioma cells used in this study allows detection of even very small populations of cells throughout the tissue sections using laser-scanning confocal microscopy, providing a comprehensive picture of the effects of administration of rVSV in the rat glioma model.

Rat survival following the administration of rVSV is also determined. Rats are treated with either PBS or VSV according to Table 5.

TABLE 5

| | # | # Cells | Rx at Day | Incubation |
|---|---|---|---|---|
| Group | Animals | InjectedH | 10 = H | Time [symbol] |
| Control Rx Group | 20 | 1 × 10⁵ | Sterile PBS | 17 days = |
| Treatment Group + | 20 | 1 × 10⁵ | Sterile PBS | 17 days = |
| IFN − βΦ | | | | |
| Treatment Group + | 20 | 1 × 10⁵ | VSV at X dose* | 17 days = |
| VSV | | | | |
| Treatment Group, | 20 | 1 × 10⁵ | VSV at X dose* | 17 days = |
| VSV + IFN − βΦ | | | | |

*Dose or time point will be determined, pending results of a pilot experiment,
= Refers to time from initial inoculation of tumor cells, [symbol]
Refers to the time point at which analyses will take place, HTumor cells, Control Rx, and VSV Rx will administered intracranially in a volume of 10 μL,
ΦA protocol for VSV protection using IFN α/βpretreatment will be developed based on the results of the pilot.

Animals persist indefinitely until such time when the tumor overcomes the animal or it become clear the animal will survive. At that time, all animals are sacrificed and analyzed as described above for the presence of any remnant of tumor. Overall survival of animals treated with VSV is significantly increased, as compared to controls.

1. A recombinant non-cytopathic Rhabdovirus comprising a nucleic acid of a Rhabdoviral genome wherein said Rhabdoviral genome comprises a deletion or a mutation within a region encoding a matrix protein (M).

2. The recombinant non-cytopathic Rhabdovirus of claim 1, further comprising a deletion or a mutation within a region encoding a glycoprotein (G).

3. The recombinant non-cytopathic Rhabdovirus of claim 1, further comprising a regulatory element.

4. The recombinant non-cytopathic Rhabdovirus of claim 1, wherein said deletion or mutation is in a region encoding the N-terminal half of said matrix protein.

5. The recombinant non-cytopathic Rhabdovirus of claim 4, wherein said deletion or mutation is in the region encoding the N-terminal part of said matrix protein encoding a nuclear localization sequence (NLS).

6. The recombinant non-cytopathic Rhabdovirus of claim 5, wherein said mutation encodes for the substitution of:
(a) An alanine amino acid residue for a methionine amino acid residue, at position 33 or 51; or
(b) A serine amino acid residue for a glycine amino acid residue, at position 226.

7. The recombinant non-cytopathic Rhabdovirus of claim 1, further comprising an insertion of a heterologous nucleic acid encoding a second polypeptide.

8. The recombinant non-cytopathic Rhabdovirus of claim 7, wherein said second polypeptide is a therapeutic polypeptide.

9. The recombinant non-cytopathic Rhabdovirus of claim 7, wherein said second polypeptide is immunogenic.

10. The recombinant non-cytopathic Rhabdovirus of claim 1, further comprising an insertion of a heterologous nucleic acid encoding a marker polypeptide.

11. The recombinant non-cytopathic Rhabdovirus of claim 10, wherein said marker polypeptide is green fluorescent protein (GFP), secreted alkaline phosphotase, DS-Red fluorescent protein, beta-galactosidase, or luciferase.

12. The recombinant non-cytopathic Rhabdovirus of claim 1, further comprising an insertion of a heterologous nucleic acid encoding a suicide gene.

13. The recombinant non-cytopathic Rhabdovirus of claim 1, further comprising an insertion of a heterologous nucleic acid encoding a cytokine gene.

14. The recombinant non-cytopathic Rhabdovirus of claim 13, wherein said cytokine is interleukin 2, interleukin 4, interleukin 12 or interferon-γ.

15. The recombinant non-cytopathic Rhabdovirus of claim 1, further comprising a Rhabdovirus G stem polypeptide.

16. The recombinant non-cytopathic Rhabdovirus of claim 1, wherein said recombinant non-cytopathic Rhabdovirus is being used as a gene delivery vector or a vaccine.

17. The recombinant non-cytopathic Rhabdovirus of claim 1, wherein said Rhabdovirus is preferentially cytopathic to neoplastic cells.

18. The recombinant non-cytopathic Rhabdovirus of claim 1, wherein said Rhabdovirus genome is a vesicular stomatitis virus (VSV) genome.

19. A method of producing a non-cytopathic recombinant Rhabdovirus comprising a genetically modified nucleic acid encoding Rhabdovirus proteins including a deletion or a mutation within a matrix protein (M) comprising the steps of: (A) inserting into a suitable cell a polynucleotide sequence encoding Rhabdovirus proteins including a deletion or a mutation within the matrix protein (M), a polynucleotide sequence encoding a marker polypeptide and a polycistronic cDNA comprising at least the 3′ and 5′ Rhabdovirus leader and trailer regions containing the cis acting signals for Rhabdovirus replication; (B) culturing the cell under conditions that select for a noncytopathic phenotype of said cell; (C) culturing said cell under conditions that permit production of the recombinant Rhabdovirus, and (D) isolating said non cytopathic recombinant Rhabdovirus.

20. The method of claim 19, wherein said non-cytopathic recombinant Rhabdovirus further comprises a heterologous nucleic acid sequence encoding a second polypeptide.

21. The method of claim 20, wherein said second polypeptide is a therapeutic polypeptide.

22. The method of claim 21, wherein said second polypeptide is immunogenic.

23. The method of claim 19, further comprising the step of isolating genomic RNA from said isolated non-cytopathic recombinant Rhabdovirus.

24. The method of claim 23, wherein said step of isolating genomic RNA is performed by using RT-PCR.

25. The method of claim 19, wherein said suitable cell, being selected from the group consisting of rodent, primate and human cells.

26. The method of claim 19, wherein said deletion or mutation resides in the N-terminal region of said matrix protein.

27. The method of claim 26, wherein said deletion or mutation residing in said N-terminal region of said matrix protein is part of a nuclear localization sequence (NLS).

28. The method of claim 19, wherein said mutation is an amino acid substitution of:
(a) An alanine amino acid residue for a methionine amino acid residue, at position 33 or 51; or
(b) A serine amino acid residue for a glycine amino acid residue, at position 226.

29. The method of claim 19, wherein the non-cytopathic recombinant Rhabdovirus is a vesicular stomatitis virus (VSV).

30. An isolated nucleic acid molecule comprising a polynucleotide sequence encoding a genome of a non-cytopathic Rhabdovirus, said polynucleotide sequence having a deletion or a mutation in a gene encoding a matrix protein (M).

31. The isolated nucleic acid molecule of claim 30, wherein said genome of a non-cytopathic Rhabdovirus further comprises a genetically modified glycoprotein (G).

32. The isolated nucleic acid molecule of claim 30, further comprising a regulatory element.

33. The isolated nucleic acid molecule of claim 30, wherein said deletion or mutation resides in a region encoding an N-terminal region of said matrix protein.

34. The isolated nucleic acid molecule of claim 33, wherein said deletion or mutation reside in the region encoding an N-terminal region of said matrix protein encoding a nuclear localization sequence (NLS).

35. The isolated nucleic acid molecule of claim 30, wherein said mutation encodes:
(a) An alanine amino acid residue for a methionine amino acid residue, at position 33 or 51; or
(b) A serine amino acid residue for a glycine amino acid residue, at position 226.

36. The isolated nucleic acid molecule of claim 30, further comprising an insertion of a heterologous nucleic acid sequence encoding a second polypeptide.

37. The isolated nucleic acid molecule of claim 36, wherein said second polypeptide is a therapeutic polypeptide.

38. The isolated nucleic acid molecule of claim 36, wherein said second polypeptide is immunogenic.

39. The isolated nucleic acid molecule of claim 30, further comprising an insertion of a heterologous nucleic acid sequence encoding a marker polypeptide.

40. The isolated nucleic acid molecule of claim 39, wherein said marker polypeptide is green fluorescent protein, secreted alkaline phosphotase, DS-Red fluorescent protein, beta-galactosidase, or luciferase.

41. The isolated nucleic acid molecule of claim 30, further comprising an insertion of a nucleic acid sequence encoding a suicide gene.

42. The isolated nucleic acid molecule of claim 30, further comprising a Rhabdovirus G stem polypeptide.

43. The isolated nucleic acid molecule of claim 30, wherein said genome is a vesicular stomatitis virus (VSV) genome.

44. A vector comprising the isolated nucleic acid molecule of claim 30.

45. A recombinant Rhabdovirus comprising a nucleic acid of a Rhabdoviral genome wherein said Rhabdoviral genome comprises a deletion or a mutation within a region encoding a membrane-proximal ectodomain of a Rhabdoviral glycoprotein (G).

46. The recombinant Rhabdovirus of claim 45, wherein said mutation encodes for the substitution of:
(i) An alanine amino acid residue for a tryptophan amino acid residue.
(ii) An alanine amino acid residue for a glutamic acid, glycine and/or phenylalanine amino acid residue; or
(iii) Aspartic acid and alanine amino acid residues for a glutamic acid, glycine or phenylalanine amino acid residue, or combinations thereof; or
(iv) Any combination of the substitutions in (a)-(c).

47. The recombinant Rhabdovirus of claim 45, wherein said mutation encodes for the deletion of:
(a) nucleotides encoding for the amino acid residues 449-461, or a fragment thereof; or
(b) nucleotides encoding for the amino acid residues 440-449, or a fragment thereof.

48. The recombinant Rhabdovirus of 45, wherein said mutation is an insertion of the nucleotides encoding for the amino acid residues 311-319 of decay acceleration factor (DAF), inserted between serine amino acid residues of the Rhabdoviral glycoprotein membrane proximal ectodomain.

49. The recombinant Rhabdovirus of claim 45, further comprising an insertion of a heterologous nucleic acid sequence encoding a second polypeptide.

50. The recombinant Rhabdovirus of claim 49, wherein said second polypeptide is a therapeutic polypeptide.

51. The recombinant Rhabdovirus of claim 49, wherein said second polypeptide is immunogenic.

52. The recombinant Rhabdovirus of 45, further comprising an insertion of a heterologous nucleic acid sequence encoding a marker polypeptide.

53. The recombinant Rhabdovirus of claim 52, wherein said marker polypeptide is green fluorescent protein (GFP), secreted alkaline phosphotase, DS-Red fluorescent protein, beta-galactosidase, or luciferase.

54. The recombinant Rhabdovirus of claim 45, further comprising an insertion of a heterologous nucleic acid sequence encoding a suicide gene.

55. The recombinant Rhabdovirus of claim 45, further comprising an insertion of a heterologous nucleic acid sequence encoding a cytokine gene.

56. The recombinant Rhabdovirus of claim 55, wherein said cytokine is interleukin 2, interleukin 4, interleukin 12 or interferon-γ.

57. The recombinant Rhabdovirus of claim 45, further comprising a deletion or a mutation within the region encoding a matrix protein (M).

58. The recombinant Rhabdovirus of claim 57, wherein said genetically modified matrix protein comprises a mutation in an N-terminal region of said matrix protein.

59. The recombinant Rhabdovirus of claim 73, wherein said deletion or mutation in said N-terminal region of said matrix protein is part of a nuclear localization sequence (NLS).

60. The recombinant Rhabdovirus of claim 45, further comprising a regulatory element.

61. The recombinant Rhabdovirus of claim 45, wherein said recombinant Rhabdovirus is being used as a gene delivery vector.

62. The recombinant Rhabdovirus of claim 45, said recombinant Rhabdovirus is being used as a vaccine.

63. The recombinant Rhabdovirus of claim 56, wherein said Rhabdovirus genome is a vesicular stomatitis virus (VSV) genome.

64. A method of producing a recombinant Rhabdovirus comprising a genetically modified nucleic acid encoding Rhabdovirus proteins including a deletion or a mutation within a membrane-proximal ectodomain of a glycoprotein (G) comprising the steps of: (A) inserting into a suitable cell a polynucleotide sequence encoding Rhabdovirus proteins including a deletion or a mutation within the membrane-proximal ectodomain of the glycoprotein (G), a polynucleotide sequence encoding a marker polypeptide and a polycistronic cDNA comprising at least the 3′ and 5′ Rhabdovirus leader and trailer regions containing the cis acting signals for Rhabdovirus replication; (B) culturing the cell under conditions that permit production of the recombinant Rhabdovirus, and (C) isolating said recombinant Rhabdovirus.

65. The method of claim 64, further comprising the step of inserting a heterologous nucleic acid sequence encoding a second polypeptide into said cell.

66. The method of claim 64, wherein said second polypeptide is a therapeutic polypeptide.

67. The method of 64, wherein said second polypeptide is immunogenic.

68. The method of claim 64, further comprising the step of isolating genomic RNA from said isolated non-cytopathic recombinant Rhabdovirus.

69. The method of claim 68, wherein said step of isolating genomic RNA is performed by using RT-PCR.

70. The method of claim 64, wherein said suitable cell, being selected from the group consisting of rodent, primate and human cells.

71. The method of claim 64, wherein said mutation of a membrane-proximal ectodomain of the glycoprotein (G) encodes for the substitution of:
(a) An alanine amino acid residue for a tryptophan amino acid residue.
(b) An alanine amino acid residue for a glutamic acid, glycine and/or phenylalanine amino acid residue; or
(c) Aspartic acid and alanine amino acid residues for a glutamic acid, glycine or phenylalanine amino acid residue, or combinations thereof; or
(d) Any combination of the substitutions in (a)-(c).

72. The method of claim 64, wherein said mutation is a deletion of the:
(a) nucleotides encoding for the amino acid residues 449-461, or a fragment thereof; or
(b) nucleotides encoding for the amino acid residues 440-449, or a fragment thereof.

73. The method of claim 64, wherein said mutation is an insertion of the nucleotides encoding for the amino acid residues 311-319 of decay acceleration factor (DAF) inserted between serine amino acid residues of the Rhabdoviral glycoprotein membrane proximal ectodomain.

74. The method of claim 64, wherein the non-cytopathic recombinant Rhabdovirus is a vesicular stomatitis virus (VSV).

75. An isolated nucleic acid molecule comprising a polynucleotide sequence encoding a genome of a Rhabdovirus, said polynucleotide sequence having a deletion or a mutation in a gene encoding a membrane-proximal ectodomain of the glycoprotein (G).

76. The isolated nucleic acid molecule of claim 75, wherein said mutation of a membrane-proximal ectodomain of the glycoprotein (G), comprises substitution of:
(a) An alanine amino acid residue for a tryptophan amino acid residue.
(b) An alanine amino acid residue for a glutamic acid, glycine and/or phenylalanine amino acid residue; or
(c) Aspartic acid and alanine amino acid residues for a glutamic acid, glycine or phenylalanine amino acid residue, or combinations thereof; or
(d) Any combination of the substitutions in (a)-(c).

77. The isolated nucleic acid molecule of claim 75, wherein said mutation is a deletion of the:
(a) nucleotides encoding for the amino acid residues 449-461, or a fragment thereof, or
(b) nucleotides encoding for the amino acid residues 440-449, or a fragment thereof.

78. The isolated nucleic acid molecule of claim 75, wherein said mutation is an insertion of the nucleotides encoding for the amino acid residues 311-319 of decay acceleration factor (DAF) inserted between serine amino acid residues of the Rhabdoviral glycoprotein membrane proximal ectodomain.

79. The isolated nucleic acid molecule of claim 75, wherein said genome of a non-cytopathic Rhabdovirus further comprises a genetically modified matrix protein (M).

80. The isolated nucleic acid molecule of claim 75, wherein said deletion or mutation is in a region encoding the N-terminus of said matrix protein.

81. The isolated nucleic acid molecule of claim 80, wherein said deletion or mutation is in a region encoding a nuclear localization sequence (NLS).

82. The isolated nucleic acid molecule of claim 75, wherein said mutation encodes for the substitution of:
(a) An alanine amino acid residue for a methionine amino acid residue, at position 33 or 51; or
(b) A glycine amino acid residue for a serine amino acid residue, at position 226.

83. The isolated nucleic acid molecule of claim 75, further comprising a regulatory element.

84. The isolated nucleic acid molecule of claim 75, further comprising an insertion of a heterologous nucleic acid sequence encoding a second polypeptide.

85. The isolated nucleic acid molecule of claim 84, wherein said second polypeptide is a therapeutic polypeptide or is immunogenic.

86. The isolated nucleic acid molecule of claim 75, further comprising an insertion of a heterologous nucleic acid sequence encoding a marker polypeptide.

87. The isolated nucleic acid molecule of claim 86, wherein said marker polypeptide is green fluorescent protein, secreted alkaline phosphotase, DS-Red fluorescent protein, beta-galactosidase, or luciferase.

88. The isolated nucleic acid molecule of claim 75, further comprising an insertion of a nucleic acid sequence encoding a suicide gene.

89. The isolated nucleic acid molecule of claim 75, further comprising a fusion facilitating polypeptide or an antireceptor.

90. The isolated nucleic acid molecule of claim 75, wherein said genome is a vesicular stomatitis virus (VSV) genome.

91. A vector comprising the isolated nucleic acid molecule of claim 75.

92. A method for treating a subject suffering from a disease associated with a defective gene comprising the step of administering to a target cell of said subject a therapeutically effective amount of a recombinant non-cytopathic Rhabdovirus, wherein the genome of said Rhabdovirus includes a deletion or a mutation within a region encoding a matrix protein (M) and/or a and a heterologous gene capable of being expressed inside the target cell, thereby treating the disease.

93. The method of claim 92, wherein said target cell is an epithelial cell, a lung cell, a kidney cell, a liver cell, an astrocyte, an immune cell, a glial cell, a prostate cell, or alpha, beta or delta cells of pancreatic islet, or acinar cells.

94. A method for immunizing a subject against a disease comprising the step of contacting a target cell of said subject with a therapeutically effective amount of a recombinant virus, wherein the virus comprises a Rhabdoviral genome, or fragment thereof, said Rhabdoviral genome or fragment thereof including a deletion or a mutation within a region encoding a matrix protein (M), and/or a deletion or a mutation within a region encoding the membrane-proximal ectodomain of a glycoprotein (G) and a heterologous gene encoding an immunogenic protein, or peptide fragment, capable of being expressed inside the target cell, thereby immunizing against a disease.

95. The method of claim 94, wherein said target cell is an epithelial cell, a lung cell, a kidney cell, a liver cell, an astrocyte, a glial cell, a prostate cell, a professional antigen presenting cell, a lymphocyte or an M cell.

96. A method for treating a subject suffering from a disease comprising the step of contacting a target cell of said subject with a therapeutically effective amount of a recombinant virus, wherein the virus comprises a Rhabdoviral genome, or fragment thereof, said Rhabdoviral genome or fragment thereof including a deletion or a mutation within a region encoding a matrix protein (M) and/or a deletion or a mutation within a region encoding the membrane-proximal ectodomain of a glycoprotein (G) and a heterologous gene encoding an immunogenic protein or peptide fragment, capable of being expressed inside the target cell, thereby treating a disease.

97. The method of claim 96, wherein said target cell is an epithelial cell, a lung cell, a kidney cell, a liver cell, an astrocyte, a glial cell, a prostate cell, a professional antigen presenting cell, a lymphocyte or an M cell.

98. A method for treating a subject suffering from a disease associated with a defective gene comprising the step of contacting a target cell of said subject with a therapeutically effective amount of a recombinant virus, wherein the virus comprises a Rhabdoviral genome, or fragment thereof, said Rhabdoviral genome or fragment thereof including a deletion or a mutation within a region encoding a matrix protein (M) and/or a deletion or a mutation within a region encoding the membrane-proximal ectodomain of a glycoprotein (G) and a heterologous gene capable of being expressed inside the target cell, thereby treating the disease.

99. The method of claim 98, wherein said target cell is an epithelial cell, a lung cell, a kidney cell, a liver cell, an astrocyte, a glial cell or a prostate cell.

100. A method for cancer cell lysis, comprising the steps of contacting a cancerous cell with a recombinant Rhabdovirus, wherein said Rhabdovirus comprises (a) a nucleic acid comprising a Rhabdoviral genome, or fragment thereof, wherein said Rhabdoviral genome or fragment thereof comprises a deletion or a mutation within a region encoding a matrix protein (M) and/or a deletion or a mutation within a region encoding the membrane-proximal ectodomain of a Rhabdoviral glycoprotein (G); and (b) a non-Rhabdoviral nucleic acid.

101. The method of claim 100, wherein said non-Rhabdoviral nucleic acid encodes for a cytokine or suicide gene.

102. A method for treating cancer, comprising the steps of contacting a cancerous cell with a recombinant virus, wherein said virus comprises (a) a nucleic acid comprising a Rhabdoviral genome, or fragment thereof, said Rhabdoviral genome or fragment thereof comprises a deletion or a mutation within a region encoding a matrix protein (M) and/or a deletion or a mutation within a region encoding the membrane-proximal ectodomain of a glycoprotein (G); and (b) a non-Rhabdoviral nucleic acid.

103. The method of claim 102, wherein said non-Rhabdoviral nucleic acid encodes for a cytokine or suicide gene.

104. A method for identifying an agent that has oncolytic activity, comprising the steps of: obtaining vibrotome slices of corona, substantia negra and cortex tissue, culturing said slices on coverslips under conditions maintaining viability and inhibiting mitosis, inoculating said slice culture with labeled cancer cells, culturing said inoculated culture with a candidate agent, and determining cancer cell viability, wherein a decrease in cancer cell viability indicates that the candidate agent is oncolytic, thereby identifying an agent that has oncolytic activity.

105. The method of claim 104, wherein said cancerous cells are of neuronal origin.

106. The method of claim 105, wherein said neuronal origin cancerous cells are glioma cells.

107. The method of claim 104, wherein said cancerous cells are labeled with a fluorescent, luminescent, chromogenic or electron dense label.

108. The method of claim 104, further comprising the step of inoculating said slice culture with labeled recombinant Rhabdovirus.

109. The method of claim 104, further comprising the step of culturing said inoculated slice culture with a cytokine.

110. The method of claim 109, wherein said cytokine is an interferon.

111. The method of claim 104, wherein culturing said slices on coverslips under conditions maintaining viability is in a medium comprising Gey's/dextrose solution, plasma, thrombin, Eagle's basal medium, Hanks' balanced salt solution, L-glutamine, or any combination thereof.

112. The method of claim 104, wherein culturing said slices on coverslips under conditions inhibiting mitosis is in a medium comprising cytosine-a-D-.arabinofuranoside, uridine, 5-fluro-2′-deoxyuridine, Gey's/dextrose solution, plasma, thrombin, Eagle's basal medium, Hanks' balanced salt solution, L-glutamine or any combination thereof.


SEQUENCE LISTING (23 sequences)

SEQ ID NO: 1 - protein, 229 aa, Vesicular stomatitis virus
MSSLKKILGLKGKGKKSKKLGIAPPPYEEDTSAEYAPSAPIDKSYFGVDEMDTYDPNQLRYEKFFFTVKMTVRSNRPFRTYSDVAAAVSHWDHMYIGMAGKRPFYKILAFLGSSNLKATPAVLADQGQPEYHTHCEGRAYLPHRMGKTPPMLNVPEHFRRPFNIGLYKGTIELTMTIYDDESLEAAPMIWDHFNSSKFSDFREKALMFGLIVEKKASGAWVLDSISHFK

SEQ ID NO: 2 - protein, 229 aa, Vesicular stomatitis virus
MSSLKKILGLKGKGKKSKKLGIAPPPYEEDTSMEYAPSAPIDKSYFGVDEADTYDPNQLRYEKFFFTVKMTVRSNRPFRTYSDVAAAVSHWDHMYIGMAGKRPFYKILAFLGSSNLKATPAVLADQGQPEYHTHCEGRAYLPHRMGKTPPMLNVPEHFRRPFNIGLYKGTIELTMTIYDDESLEAAPMIWDHFNSSKFSDFREKALMFGLIVEKKASGAWVLDSISHFK

SEQ ID NO: 3 - protein, 229 aa, Vesicular stomatitis virus
MSSLKKILGLKGKGKKSKKLGIAPPPYEEDTSMEYAPSAPIDKSYFGVDEMDTYDPNQLRYEKFFFTVKMTVRSNRPFRTYSDVAAAVSHWDHMYIGMAGKRPFYKILAFLGSSNLKATPAVLADQGQPEYHAHCEGRAYLPHRMGKTPPMLNVPEHFRRPFNIGLYKGTIELTMTIYDDESLEAAPMIWDHFNSSKFSDFREKALMFGLIVEKKASGAWVLDSISHFK

SEQ ID NO: 4 - protein, 229 aa, Vesicular stomatitis virus
MSSLKKILGLKGKGKKSKKLGIAPPPYEEDTSMEYAPSAPIDKSYFGVDEMDTYDPNQLRYEKFFFTVKMTVRSNRPFRTYSDVAAAVSHWDHMYIGMAGKRPFYKILAFLGSSNLKATPAVLADQGQPEYHTHCEGRAYLPHRMGKTPPMLNVPEHFRRPFNIGLYKGTIELTMTIYDDESLEAAPMIWDHFNSSKFSDFREKALMFGLIVEKKASGAWVLDSIGHFK

SEQ ID NO: 5 - DNA, 690 bp, Vesicular stomatitis virus
atgagttccttaaagaagattctcggtctgaaggggaaaggtaagaaatctaagaaattagggatcgcaccacccccttatgaagaggacactagcgctgagtatgctccgagcgctccaattgacaaatcctattttggagttgacgaggctgacacctatgatccgaatcaattaagatatgagaaattcttctttacagtgaaaatgacggttagatctaatcgtccgttcagaacatactcagatgtggcagccgctgtatcccattgggatcacatgtacatcggaatggcagggaaacgtcccttctacaaaatcttggcttttttgggttcttctaatctaaaggccactccagcggtattggcagatcaaggtcaaccagagtatcacgctcactgcgaaggcagggcttatttgccacataggatggggaagacccctcccatgctcaatgtaccagagcacttcagaagaccattcaatataggtctttacaagggaacgattgagctcacaatgaccatctacgatgatgagtcactggaagcagctcctatgatctgggatcatttcaattcttccaaattttctgatttcagagagaaggccttaatgtttggcctgattgtcgagaaaaaggcatctggagcgtgggtcctggactctatcggccacttcaaatga

SEQ ID NO: 6 - protein, 42 aa, Vesicular stomatitis virus
FEHPHIQDAASQLPDDESLFFGDTGLSKNPIALVEGWFSSWK

SEQ ID NO: 7 - protein, 42 aa, Vesicular stomatitis virus
FEHPHIQDAASQLPDDESLFFGDTGLSKNPIELVEDWFSSWK

SEQ ID NO: 8 - protein, 42 aa, Vesicular stomatitis virus
FEHPHIQDAASQLPDDESLFFGDTGLSKNPIELVEGAFSSWK

SEQ ID NO: 9 - protein, 42 aa, Vesicular stomatitis virus
FEHPHIQDAASQLPDDESLFFGDTGLSKNPIELVEGWASSWK

SEQ ID NO: 10 - protein, 42 aa, Vesicular stomatitis virus
FEHPHIQDAASQLPDDESLFFGDTGLSKNPIELVEGWFSSAK

SEQ ID NO: 11 - protein, 42 aa, Vesicular stomatitis virus
FEHPHIQDAASQLPDDESLFFGDTGLSKNPIELVEGWFSSWK

SEQ ID NO: 12 - protein, 42 aa, Vesicular stomatitis virus
FEHPHIQDAASQLPDDESLFFGDTGLSKNPIELVEAWFSSAK

SEQ ID NO: 13 - protein, 42 aa, Vesicular stomatitis virus
FEHPHIQDAASQLPDDESLFFGDTGLSKNPIELVEAAFSSAK

SEQ ID NO: 14 - protein, 42 aa, Vesicular stomatitis virus
FEHPHIQDAASQLPDDESLFFGDTGLSKNPIELVEDAFSSAK

SEQ ID NO: 15 - protein, 35 aa, Vesicular stomatitis virus
FEHPHIQDAASQLPDDESLFFGDTGLSKNPIEKSS

SEQ ID NO: 16 - protein, 32 aa, Vesicular stomatitis virus
FEHPHIQDAASQLPDDESLPIELVEGWFSSWK

SEQ ID NO: 17 - protein, 54 aa, Vesicular stomatitis virus
FEHPHIQDAASQLPDDESLFFGDTGLSKNPIELVEGWFSSWKSSSPNKGSGTTS

SEQ ID NO: 18 - protein, 45 aa, Vesicular stomatitis virus
FEHPHIQDAASQLPDDESLFFGDTGLSKNPIEKSSSPNKGSGTTS

SEQ ID NO: 19 - protein, 6 aa, Vesicular stomatitis virus
FFGDTG

SEQ ID NO: 20 - protein, 29 aa, Vesicular stomatitis virus
FEHPHIQDAASQLPDDESLFFGDTGLSKK

SEQ ID NO: 21 - protein, 53 aa, Vesicular stomatitis virus
FEHPHIQDAASQLPDDESLFFGDTGLSKNPIELVEGWFSSWKSSSTVIHADNP

SEQ ID NO: 22 - protein, 42 aa, Vesicular stomatitis virus
FEHPHIQDAASQLPDDESLFFGDTGLSKNPASSFWGEVLEIK

SEQ ID NO: 23 - protein, 42 aa, Vesicular stomatitis virus
FEHPHIQDAASQLPDDESLFFGDTGLSKNPASSFAGEVLEIK